SEQUENCE LISTING (99 sequences)

SEQ ID NO: 1 - protein, 116 aa, Homo sapiens
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAG

SEQ ID NO: 2 - protein, 116 aa, Homo sapiens
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAA

SEQ ID NO: 3 - protein, 116 aa, synthetic construct
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYAKALVELKVAA

SEQ ID NO: 4 - protein, 513 aa, synthetic construct
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYAKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 5 - DNA, 348 bp, synthetic construct
  source 1..348: mol_type = other DNA
caattcatcgtcgtgggccccaccgatcctatcctggctacagtgggcgagaacaccacactgagatgccatctgagccccgagaagaacgccgaggatatggaagtgcggtggttcagaagccagttcagccctgccgtgttcgtgtacaaaggcggcagagagcggaccgaggaacagatggaagagtacagaggccggaccaccttcgtgtccaaggacatctctagaggcagcgtggccctggtcatccacaacatcacagcccaagagaacggcacctacaggtgctacttccaagagggcagaagctacgacgaggccatcctgcatctggtggttgccgga

SEQ ID NO: 6 - DNA, 348 bp, synthetic construct
  source 1..348: mol_type = other DNA
caattcagcgtgctgggcccttctggacctatcctggctatggttggagaggacgccgacctgccttgccacctgtttccaacaatgagcgccgagacaatggaactgaaatgggtgtccagcagcctgcggcaggtcgtgaatgtgtacgccgatggcaaagaggtggaagatagacagagcgccccttacagaggccggaccagcattctgagagatggcatcacagccggcaaggccgctctgagaatccacaatgtgaccgccagcgacagcggcaagtacctgtgctacttccaagacggcgacttctacgagaaggccctggtcgagctgaaagtggctgcc

SEQ ID NO: 7 - DNA, 348 bp, synthetic construct
  source 1..348: mol_type = other DNA
caattcagcgtgctgggcccttctggacctatcctggctatggttggagaggacgccgacctgccttgccacctgtttccaacaatgagcgccgagacaatggaactgaaatgggtgtccagcagcctgcggcaggtcgtgaatgtgtacgccgatggcaaagaggtggaagatagacagagcgccccttacagaggccggaccagcattctgagagatggcatcacagccggcaaggccgctctgagaatccacaatgtgaccgccagcgacagcggcaagtacctgtgctacttccaagacggcgacttctacgcgaaggccctggtcgagctgaaagtggctgcc

SEQ ID NO: 8 - DNA, 1539 bp, synthetic construct
  source 1..1539: mol_type = other DNA
atgaagatggccagctttctggccttcctgctgctgaacttcagagtgtgtctgctgctgctccagctgctgatgcctcacagcgcccagttttctgtgctgggcccttctggacctatcctggccatggtgggagaagatgccgacctgccttgccatctgttccctaccatgagcgccgagacaatggaactgaaatgggtgtccagcagcctgcggcaggtcgtgaatgtgtacgccgatggcaaagaggtggaagatagacagagcgccccttacagaggccggaccagcattctgagagatggcatcacagccggcaaggccgctctgagaatccacaatgtgaccgccagcgacagcggcaagtacctgtgctacttccaagacggcgacttctacgcgaaggccctggtcgagctgaaagtggctgccctgggatctgatctgcacgtggacgtgaagggctacaaggatggcggcatccacctggaatgtagaagcacaggatggtacccccagcctcagatccagtggtccaacaacaagggcgagaacatccccaccgtggaagctccagttgtggctgatggcgtgggactgtatgccgtggccgcctctgtgatcatgagaggctctagcggagagggcgtgtcctgcacaatcagatctagcctgctgggcctcgaaaagaccgcctccatctctatcgccgatccattctttcggagcgcccagagatggatcgctgctctggctggaacactgcccgttctgctgttgcttcttggcggagccggctactttctgtggcagcagcaagaggaaaagaaaacccagttccggaagaagaagcgcgagcaagagctgcgcgaaatggcctggtccaccatgaagcaagagcagtccaccagagtgaagctgctggaagaactgcggtggcggagcatccagtatgcatctcggggagagagacattcagcctataatgaatggaaaaaggccctcttcaagcctgcggatgtgattctggatccaaaaacagcaaaccccatcctccttgtttctgaggaccagaggagtgtgcagcgtgccaaggagccccaggatctgccagacaaccctgagagatttaattggcattattgtgttctcggctgtgagagcttcatatcagggagacattactgggaggtggaggtaggggacaggaaagagtggcatataggggtgtgcagtaagaatgtgcagagaaaaggctgggtcaaaatgacacctgagaatggattctggactatggggctgactgatgggaataagtatcggactctaactgagcccagaaccaacctgaaacttcctaagccccctaagaaagtgggggtcttcctggactatgagactggagatatctcattctacaatgctgtggatggatcgcatattcatactttcctggacgtctccttctctgaggctctatatcctgttttcagaattttgaccttggagcccacggccctgactatttgtccagcg

SEQ ID NO: 9 - protein, 499 aa, Homo sapiens
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 10 - protein, 528 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPLAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSGPDTSQSGDPPEPIESIPWSHSHVDKPWSFQQPPHNTHLPAASFTPTTDLSPSFLLLTRLCF

SEQ ID NO: 11 - protein, 375 aa, Homo sapiens
MRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 12 - protein, 334 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAELQFFSN

SEQ ID NO: 13 - protein, 466 aa, Homo sapiens
MEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 14 - protein, 330 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAGPV

SEQ ID NO: 15 - protein, 484 aa, Homo sapiens
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 16 - protein, 352 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPGEEMLQMRLHFVK

SEQ ID NO: 17 - protein, 378 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPGPPIGQTQQQTRGQGSPVALSQESAQRTDSWGPEEGGES

SEQ ID NO: 18 - protein, 461 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 19 - DNA, 1503 bp, synthetic construct
  source 1..1503: mol_type = other DNA
cagtttattgtcgtgggcccgaccgatcctattctcgctacagtgggcgaaaatacaacacttaggtgtcatttgtctccggagaaaaacgctgaggatatggaggtaaggtggtttagaagtcaattcagtccggctgttttcgtttataaaggcggtagagaacgaactgaagaacaaatggaagagtatcgaggacgcacaacgttcgttagcaaagacattagcaggggctctgtggcgttggttatccacaacattactgcccaagagaatggtacatataggtgttactttcaagaaggaaggtcatatgatgaggcaattcttcatttggtggttgcgggactgggctccaaaccgctgatctccatgagaggccatgaggacgggggtatccgactggagtgcatatctcggggctggtatcccaagccactcacagtctggagggatccctacggtggggtcgccccagcattgaaggaagtttctatgccagacgccgacggcctgtttatggttaccaccgcggtgattatacgcgacaaatcagtacgcaatatgagttgctccatcaataatactctgctcggtcagaaaaaggagagtgtcatctttatcccagaatctttcatgcccagcgtaagcccatgtgctgtagcgctgcctatcatagtagtaatcctgatgattcccatagcagtctgtatttactggataaataagttgcagaaagaaaagaaaatactctctggggagaaggagttcgagcgcgaaaccagagagatcgctcttaaggaattggaaaaggaaagggtgcagaaggaggaagaactccaggtaaaggagaaattgcaagaagaactccggtggcgccgcacatttctccatgccgtcgatgtagtcctggacccagacactgcgcacccagacctgttcttgtccgaggatcggcgcagtgtcaggaggtgcccgttccggcatttgggtgaatctgtcccagacaatccggaaaggtttgacagtcagccgtgcgtcctcggcagagaatcatttgcttctggaaagcactactgggaggttgaagtcgagaacgtaattgaatggacggtgggtgtgtgccgggacagtgtcgagcggaaaggagaagttctgcttatcccgcagaatggattttggacgcttgaaatgcacaaaggccaatatagagccgtaagttctcccgaccgaattttgccccttaaagagagcttgtgtagagtaggtgtgttcctcgactacgaggctggcgatgtctcattctataatatgcgggataggtctcacatatacacctgtccgcgctccgcattcagtgtcccagtgcgaccattttttcgcttgggatgtgaagatagcccgatattcatatgcccagcattgactggtgccaatggcgtcaccgtcccggaagaaggtctgacccttcatagagttgggacccatcaatccctttaatga

SEQ ID NO: 20 - RNA, 1590 nt, Homo sapiens
  source 1..1590: mol_type = mRNA
atggaatcagctgctgccctgcacttctcccggccagcctccctcctcctcctcctcctcagcctgtgtgcactggtctcagcccagtttattgtcgtggggcccactgatcccatcttggccacggttggagaaaacactacgttacgctgccatctgtcacccgagaaaaatgctgaggacatggaggtgcggtggttccggtctcagttctcccccgcagtgtttgtgtataaaggtggcagagagagaacagaggagcagatggaggagtaccgaggaagaaccacctttgtgagcaaagacatcagcaggggcagcgtggccctggtcatacacaacatcacagcccaggaaaacggcacctaccgctgttacttccaagaaggcaggtcctacgatgaggccatcctgcacctcgtagtggcaggactaggctctaagcccctcatttcaatgaggggccatgaagacgggggcatccggctggagtgcatatctagagggtggtacccaaagcccctcacagtgtggagggacccctacggtggggttgcgcctgccctgaaagaggtctccatgcctgatgcagacggcctcttcatggtcaccacggctgtgatcatcagagacaagtctgtgaggaacatgtcctgctctatcaacaacaccctgctcggccagaagaaagaaagtgtcatttttattccagaatcctttatgcccagtgtgtctcccctggccgtatgcatctattggatcaacaaactccaaaaggaaaaaaagattctgtcaggggaaaaggagtttgaacgggaaacaagagaaattgctctaaaggaactggagaaagaacgtgtgcaaaaagaggaagaacttcaagtaaaagagaaacttcaagaagaattgcgatggagaagaacattcttacatgctgttgatgtggtcctggatccagacaccgctcatcccgatctcttcctgtcagaggaccggagaagtgtgagaaggtgccccttcaggcacctaggggagagcgtgcctgacaacccagagagattcgacagtcagccttgtgtcctaggccgggagagcttcgcttcagggaaacattactgggaggtggaggtggaaaacgtgattgagtggactgtgggggtctgtagagacagtgttgagaggaaaggggaggtcctgctgattcctcagaatggcttctggaccttggagatgcataaagggcaataccgggccgtgtcctcccctgataggattctccctttgaaggagtccctttgccgggtgggcgtcttcctggactatgaagctggagatgtctccttctacaacatgagggacagatcgcacatctacacatgtccccgttcagccttttccgggcctgacacttcacagagtggggacccaccagagcctatagaatcaattccttggtctcacagccatgtagacaagccctggtcatttcagcagccaccgcacaacacccatcttccagctgcctctttcacacccactacagacctcagccccagttttctcctcctcactaggctgtgtttttagtag

SEQ ID NO: 21 - RNA, 1128 nt, Homo sapiens
  source 1..1128: mol_type = mRNA
atgaggggccatgaagacgggggcatccggctggagtgcatatctagagggtggtacccaaagcccctcacagtgtggagggacccctacggtggggttgcgcctgccctgaaagaggtctccatgcctgatgcagacggcctcttcatggtcaccacggctgtgatcatcagagacaagtctgtgaggaacatgtcctgctctatcaacaacaccctgctcggccagaagaaagaaagtgtcatttttattccagaatcctttatgcccagtgtgtctccctgtgcagtggccctgcctatcattgtggttattctgatgatacccattgccgtatgcatctattggatcaacaaactccaaaaggaaaaaaagattctgtcaggggaaaaggagtttgaacgggaaacaagagaaattgctctaaaggaactggagaaagaacgtgtgcaaaaagaggaagaacttcaagtaaaagagaaacttcaagaagaattgcgatggagaagaacattcttacatgctgttgatgtggtcctggatccagacaccgctcatcccgatctcttcctgtcagaggaccggagaagtgtgagaaggtgccccttcaggcacctaggggagagcgtgcctgacaacccagagagattcgacagtcagccttgtgtcctaggccgggagagcttcgcttcagggaaacattactgggaggtggaggtggaaaacgtgattgagtggactgtgggggtctgtagagacagtgttgagaggaaaggggaggtcctgctgattcctcagaatggcttctggaccttggagatgcataaagggcaataccgggccgtgtcctcccctgataggattctccctttgaaggagtccctttgccgggtgggcgtcttcctggactatgaagctggagatgtctccttctacaacatgagggacagatcgcacatctacacatgtccccgttcagccttttccgtgcctgtgaggcccttcttcaggttggggtgtgaggacagccccatcttcatctgccctgcactcacaggagccaatggggtcacggtgcctgaagagggcctgacacttcacagagtggggacccaccagagcctatag

SEQ ID NO: 22 - RNA, 1005 nt, Homo sapiens
  source 1..1005: mol_type = mRNA
atggaatcagctgctgccctgcacttctcccggccagcctccctcctcctcctcctcctcagcctgtgtgcactggtctcagcccagtttattgtcgtggggcccactgatcccatcttggccacggttggagaaaacactacgttacgctgccatctgtcacccgagaaaaatgctgaggacatggaggtgcggtggttccggtctcagttctcccccgcagtgtttgtgtataaaggtggcagagagagaacagaggagcagatggaggagtaccgaggaagaaccacctttgtgagcaaagacatcagcaggggcagcgtggccctggtcatacacaacatcacagcccaggaaaacggcacctaccgctgttacttccaagaaggcaggtcctacgatgaggccatcctgcacctcgtagtggcaggactaggctctaagcccctcatttcaatgaggggccatgaagacgggggcatccggctggagtgcatatctagagggtggtacccaaagcccctcacagtgtggagggacccctacggtggggttgcgcctgccctgaaagaggtctccatgcctgatgcagacggcctcttcatggtcaccacggctgtgatcatcagagacaagtctgtgaggaacatgtcctgctctatcaacaacaccctgctcggccagaagaaagaaagtgtcatttttattccagaatcctttatgcccagtgtgtctccctgtgcagtggccctgcctatcattgtggttattctgatgatacccattgccgtatgcatctattggatcaacaaactccaaaaggaaaaaaagattctgtcaggggaaaaggagtttgaacgggaaacaagagaaattgctctaaaggaactggagaaagaacgtgtgcaaaaagaggaagaacttcaagtaaaagagaaacttcaagaagaattgcgatggagaagaacattcttacatgctgagctccaattcttctcaaactga

SEQ ID NO: 23 - RNA, 1401 nt, Homo sapiens
  source 1..1401: mol_type = mRNA
atggaggtgcggtggttccggtctcagttctcccccgcagtgtttgtgtataaaggtggcagagagagaacagaggagcagatggaggagtaccgaggaagaaccacctttgtgagcaaagacatcagcaggggcagcgtggccctggtcatacacaacatcacagcccaggaaaacggcacctaccgctgttacttccaagaaggcaggtcctacgatgaggccatcctgcacctcgtagtggcaggactaggctctaagcccctcatttcaatgaggggccatgaagacgggggcatccggctggagtgcatatctagagggtggtacccaaagcccctcacagtgtggagggacccctacggtggggttgcgcctgccctgaaagaggtctccatgcctgatgcagacggcctcttcatggtcaccacggctgtgatcatcagagacaagtctgtgaggaacatgtcctgctctatcaacaacaccctgctcggccagaagaaagaaagtgtcatttttattccagaatcctttatgcccagtgtgtctccctgtgcagtggccctgcctatcattgtggttattctgatgatacccattgccgtatgcatctattggatcaacaaactccaaaaggaaaaaaagattctgtcaggggaaaaggagtttgaacgggaaacaagagaaattgctctaaaggaactggagaaagaacgtgtgcaaaaagaggaagaacttcaagtaaaagagaaacttcaagaagaattgcgatggagaagaacattcttacatgctgttgatgtggtcctggatccagacaccgctcatcccgatctcttcctgtcagaggaccggagaagtgtgagaaggtgccccttcaggcacctaggggagagcgtgcctgacaacccagagagattcgacagtcagccttgtgtcctaggccgggagagcttcgcttcagggaaacattactgggaggtggaggtggaaaacgtgattgagtggactgtgggggtctgtagagacagtgttgagaggaaaggggaggtcctgctgattcctcagaatggcttctggaccttggagatgcataaagggcaataccgggccgtgtcctcccctgataggattctccctttgaaggagtccctttgccgggtgggcgtcttcctggactatgaagctggagatgtctccttctacaacatgagggacagatcgcacatctacacatgtccccgttcagccttttccgtgcctgtgaggcccttcttcaggttggggtgtgaggacagccccatcttcatctgccctgcactcacaggagccaatggggtcacggtgcctgaagagggcctgacacttcacagagtggggacccaccagagcctatag

SEQ ID NO: 24 - RNA, 993 nt, Homo sapiens
  source 1..993: mol_type = mRNA
atggaatcagctgctgccctgcacttctcccggccagcctccctcctcctcctcctcctcagcctgtgtgcactggtctcagcccagtttattgtcgtggggcccactgatcccatcttggccacggttggagaaaacactacgttacgctgccatctgtcacccgagaaaaatgctgaggacatggaggtgcggtggttccggtctcagttctcccccgcagtgtttgtgtataaaggtggcagagagagaacagaggagcagatggaggagtaccgaggaagaaccacctttgtgagcaaagacatcagcaggggcagcgtggccctggtcatacacaacatcacagcccaggaaaacggcacctaccgctgttacttccaagaaggcaggtcctacgatgaggccatcctgcacctcgtagtggcaggactaggctctaagcccctcatttcaatgaggggccatgaagacgggggcatccggctggagtgcatatctagagggtggtacccaaagcccctcacagtgtggagggacccctacggtggggttgcgcctgccctgaaagaggtctccatgcctgatgcagacggcctcttcatggtcaccacggctgtgatcatcagagacaagtctgtgaggaacatgtcctgctctatcaacaacaccctgctcggccagaagaaagaaagtgtcatttttattccagaatcctttatgcccagtgtgtctccctgtgcagtggccctgcctatcattgtggttattctgatgatacccattgccgtatgcatctattggatcaacaaactccaaaaggaaaaaaagattctgtcaggggaaaaggagtttgaacgggaaacaagagaaattgctctaaaggaactggagaaagaacgtgtgcaaaaagaggaagaacttcaagtaaaagagaaacttcaagaagaattgcgatggagaagaacattcttacatgctgggccagtttga

SEQ ID NO: 25 - DNA, 1452 bp, synthetic construct
  source 1..1452: mol_type = other DNA
cagttttctgtgcttggaccctctgggcccatcctggccatggtgggtgaagacgctgatctgccctgtcacctgttcccgaccatgagtgcagagaccatggagctgaagtgggtgagttccagcctaaggcaggtggtgaacgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagcactgggttctgatcttcacgttgatgtgaagggttacaaggatggagggatccatctggagtgcaggtccactggctggtacccccaaccccaaatacagtggagcaacaacaagggagagaacatcccgactgtggaagcacctgtggttgcagacggagtgggcctgtatgcagtagcagcatctgtgatcatgagaggcagctctggggagggtgtatcctgtaccatcagaagttccctcctcggcctggaaaagacagccagcatttccatcgcagaccccttcttcaggagcgcccagaggtggatcgccgccctggcagggaccctgcctgtcttgctgctgcttcttgggggagccggttacttcctgtggcaacagcaggaggaaaaaaagactcagttcagaaagaaaaagagagagcaagagttgagagaaatggcatggagcacaatgaagcaagaacaaagcacaagagtgaagctcctggaggaactcagatggagaagtatccagtatgcatctcggggagagagacattcagcctataatgaatggaaaaaggccctcttcaagcctgcggatgtgattctggatccaaaaacagcaaaccccatcctccttgtttctgaggaccagaggagtgtgcagcgtgccaaggagccccaggatctgccagacaaccctgagagatttaattggcattattgtgttctcggctgtgagagcttcatatcagggagacattactgggaggtggaggtaggggacaggaaagagtggcatataggggtgtgcagtaagaatgtgcagagaaaaggctgggtcaaaatgacacctgagaatggattctggactatggggctgactgatgggaataagtatcggactctaactgagcccagaaccaacctgaaacttcctaagccccctaagaaagtgggggtcttcctggactatgagactggagatatctcattctacaatgctgtggatggatcgcatattcatactttcctggacgtctccttctctgaggctctatatcctgttttcagaattttgaccttggagcccacggccctgactatttgtccagcg

SEQ ID NO: 26 - RNA, 1059 nt, Homo sapiens
  source 1..1059: mol_type = mRNA
atgaaaatggcaagtttcctggccttccttctgctcaactttcgtgtctgcctccttttgcttcagctgctcatgcctcactcagctcagttttctgtgcttggaccctctgggcccatcctggccatggtgggtgaagacgctgatctgccctgtcacctgttcccgaccatgagtgcagagaccatggagctgaagtgggtgagttccagcctaaggcaggtggtgaacgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagcactgggttctgatcttcacgttgatgtgaagggttacaaggatggagggatccatctggagtgcaggtccactggctggtacccccaaccccaaatacagtggagcaacaacaagggagagaacatcccgactgtggaagcacctgtggttgcagacggagtgggcctgtatgcagtagcagcatctgtgatcatgagaggcagctctggggagggtgtatcctgtaccatcagaagttccctcctcggcctggaaaagacagccagcatttccatcgcagaccccttcttcaggagcgcccagaggtggatcgccgccctggcagggaccctgcctgtcttgctgctgcttcttgggggagccggttacttcctgtggcaacagcaggaggaaaaaaagactcagttcagaaagaaaaagagagagcaagagttgagagaaatggcatggagcacaatgaagcaagaacaaagcacaagagtgaagctcctggaggaactcagatggagaagtatccagtatgcatctcggggagagagacattcagcctataatgaatggaaaaaggccctcttcaagcctggtgaggaaatgcttcagatgaggctccactttgttaaataa

SEQ ID NO: 27 - RNA, 1137 nt, Homo sapiens
  source 1..1137: mol_type = mRNA
atgaaaatggcaagtttcctggccttccttctgctcaactttcgtgtctgcctccttttgcttcagctgctcatgcctcactcagctcagttttctgtgcttggaccctctgggcccatcctggccatggtgggtgaagacgctgatctgccctgtcacctgttcccgaccatgagtgcagagaccatggagctgaagtgggtgagttccagcctaaggcaggtggtgaacgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagcactgggttctgatcttcacgttgatgtgaagggttacaaggatggagggatccatctggagtgcaggtccactggctggtacccccaaccccaaatacagtggagcaacaacaagggagagaacatcccgactgtggaagcacctgtggttgcagacggagtgggcctgtatgcagtagcagcatctgtgatcatgagaggcagctctggggagggtgtatcctgtaccatcagaagttccctcctcggcctggaaaagacagccagcatttccatcgcagaccccttcttcaggagcgcccagaggtggatcgccgccctggcagggaccctgcctgtcttgctgctgcttcttgggggagccggttacttcctgtggcaacagcaggaggaaaaaaagactcagttcagaaagaaaaagagagagcaagagttgagagaaatggcatggagcacaatgaagcaagaacaaagcacaagagtgaagctcctggaggaactcagatggagaagtatccagtatgcatctcggggagagagacattcagcctataatgaatggaaaaaggccctcttcaagcctgggcctcccattggccaaacccaacagcaaaccagaggacaagggagcccagtggcactgtctcaggaatctgcccagaggacagattcctggggcccagaagagggtggagaaagctga

SEQ ID NO: 28 - RNA, 1386 nt, Homo sapiens
  source 1..1386: mol_type = mRNA
atgaaaatggcaagtttcctggccttccttctgctcaactttcgtgtctgcctccttttgcttcagctgctcatgcctcactcagctcagttttctgtgcttggaccctctgggcccatcctggccatggtgggtgaagacgctgatctgccctgtcacctgttcccgaccatgagtgcagagaccatggagctgaagtgggtgagttccagcctaaggcaggtggtgaacgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagacggagtgggcctgtatgcagtagcagcatctgtgatcatgagaggcagctctggggagggtgtatcctgtaccatcagaagttccctcctcggcctggaaaagacagccagcatttccatcgcagaccccttcttcaggagcgcccagaggtggatcgccgccctggcagggaccctgcctgtcttgctgctgcttcttgggggagccggttacttcctgtggcaacagcaggaggaaaaaaagactcagttcagaaagaaaaagagagagcaagagttgagagaaatggcatggagcacaatgaagcaagaacaaagcacaagagtgaagctcctggaggaactcagatggagaagtatccagtatgcatctcggggagagagacattcagcctataatgaatggaaaaaggccctcttcaagcctgcggatgtgattctggatccaaaaacagcaaaccccatcctccttgtttctgaggaccagaggagtgtgcagcgtgccaaggagccccaggatctgccagacaaccctgagagatttaattggcattattgtgttctcggctgtgagagcttcatatcagggagacattactgggaggtggaggtaggggacaggaaagagtggcatataggggtgtgcagtaagaatgtgcagagaaaaggctgggtcaaaatgacacctgagaatggattctggactatggggctgactgatgggaataagtatcggactctaactgagcccagaaccaacctgaaacttcctaagccccctaagaaagtgggggtcttcctggactatgagactggagatatctcattctacaatgctgtggatggatcgcatattcatactttcctggacgtctccttctctgaggctctatatcctgttttcagaattttgaccttggagcccacggccctgactatttgtccagcgtga

SEQ ID NO: 29 - protein, 285 aa, synthetic construct
ETGQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSSGGLTPRGVRLGGLEIEAAFLEQENTALETEVAELEQEVQRLENIVSQYETRYGPLGTKHHHHHH

SEQ ID NO: 30 - protein, 301 aa, synthetic construct
ETGQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQSGGLTPRGVRLGGLEIRAAFLRRRNTALRTRVAELRQRVQRLRNIVSQYETRYGPLGGTLNDIFEAQKIEWHEDYKDDDDK

SEQ ID NO: 31 - DNA, 969 bp, synthetic construct
  source 1..969: mol_type = other DNA
gaattcaagcttgccaccatggggatccttcccagccctgggatgcctgcgctgctctccctcgtgagccttctctccgtgctgctgatgggttgcgtagctgaaaccggtcaattcatcgtcgtgggccccaccgatcctatcctggctacagtgggcgagaacaccacactgagatgccatctgagccccgagaagaacgccgaggatatggaagtgcggtggttcagaagccagttcagccctgccgtgttcgtgtacaaaggcggcagagagcggaccgaggaacagatggaagagtacagaggccggaccaccttcgtgtccaaggacatctctagaggcagcgtggccctggtcatccacaacatcacagcccaagagaacggcacctacaggtgctacttccaagagggcagaagctacgacgaggccatcctgcatctggtggttgccggactgggaagcaagcccctgatctctatgagaggccacgaggacggcggcatcagactggaatgtatcagcagaggctggtatcccaagccactgaccgtttggagagatccctatggcggagtggcccctgctctgaaagaagtgtctatgcccgatgccgacggcctgttcatggtcacaacagccgtgatcatccgggacaagagcgtgcggaatatgagctgcagcatcaacaacaccctgctgggccagaaaaaagagagcgtcatcttcatccccgagtccttcatgcccagcgttagttccggaggcctgacacctagaggcgttagacttggaggcctggaaatcgaggccgccttcctggaacaagagaacacagccctggaaacagaggtggccgagcttgagcaagaggtgcagcggctggaaaacatcgtgtcccagtacgagacaagatacggccctctgggtaccaagcaccaccaccatcaccactgatgactcgag

SEQ ID NO: 32 - DNA, 1017 bp, synthetic construct
  source 1..1017: mol_type = other DNA
gaattcaagcttgccaccatggggatccttcccagccctgggatgcctgcgctgctctccctcgtgagccttctctccgtgctgctgatgggttgcgtagctgaaaccggtcaattcagcgtgctgggcccttctggacctatcctggctatggttggagaggacgccgacctgccttgccacctgtttccaacaatgagcgccgagacaatggaactgaaatgggtgtccagcagcctgcggcaggtcgtgaatgtgtacgccgatggcaaagaggtggaagatagacagagcgccccttacagaggccggaccagcattctgagagatggcatcacagccggcaaggccgctctgagaatccacaatgtgaccgccagcgacagcggcaagtacctgtgctacttccaagacggcgacttctacgagaaggccctggtcgagctgaaagtggctgccctgggatctgatctgcacgtggacgtgaagggctacaaggatggcggcatccacctggaatgtagaagcaccggctggtatccccagcctcagatccagtggtccaacaacaagggcgagaacatccccaccgtggaagctccagttgtggctgatggcgtgggactgtatgccgtggccgcctctgtgatcatgagaggctctagcggagagggcgtgtcctgcacaatcagatctagcctgctgggcctcgaaaagaccgcctccatctctatcgccgatccattcttcagaagcgcccagtccggaggcctgacacctagaggtgttagacttggaggcctggaaatcagagccgccttcctgcggagaagaaacacagccctgagaaccagagtggccgagctgagacagagagtgcagcggctgagaaacatcgtgtcccagtacgagacaagatacggccctctcggcggtaccctgaacgatatctttgaggcccagaaaatcgagtggcacgaggactacaaggacgacgacgacaagtgatgactcgag

SEQ ID NO: 33 - protein, 275 aa, Homo sapiens
ETGQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSSGGLEIEAAFLEQENTALETEVAELEQEVQRLENIVSQYETRYGPLGTKHHHHHH

SEQ ID NO: 34 - protein, 291 aa, synthetic construct
ETGQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQSGGLEIRAAFLRRRNTALRTRVAELRQRVQRLRNIVSQYETRYGPLGGTLNDIFEAQKIEWHEDYKDDDDK

SEQ ID NO: 35 - DNA, 834 bp, synthetic construct
  source 1..834: mol_type = other DNA
accggtcaattcatcgtcgtgggccccaccgatcctatcctggctacagtgggcgagaacaccacactgagatgccatctgagccccgagaagaacgccgaggatatggaagtgcggtggttcagaagccagttcagccctgccgtgttcgtgtacaaaggcggcagagagcggaccgaggaacagatggaagagtacagaggccggaccaccttcgtgtccaaggacatctctagaggcagcgtggccctggtcatccacaacatcacagcccaagagaacggcacctacaggtgctacttccaagagggcagaagctacgacgaggccatcctgcatctggtggttgccggactgggaagcaagcccctgatctctatgagaggccacgaggacggcggcatcagactggaatgtatcagcagaggctggtatcccaagccactgaccgtttggagagatccctatggcggagtggcccctgctctgaaagaagtgtctatgcccgatgccgacggcctgttcatggtcacaacagccgtgatcatccgggacaagagcgtgcggaatatgagctgcagcatcaacaacaccctgctgggccagaaaaaagagagcgtcatcttcatccccgagtccttcatgcccagcgttagttccggaggcctggaaatcgaggccgccttcctggaacaagagaacacagccctggaaacagaggtggccgagcttgagcaagaggtgcagcggctggaaaacatcgtgtcccagtacgagacaagatacggccctctgggtaccaagcaccaccaccatcaccactgatgactcgag

SEQ ID NO: 36 - RNA, 987 nt, Homo sapiens
  source 1..987: mol_type = mRNA
gaattcaagcttgccaccatggggatccttcccagccctgggatgcctgcgctgctctccctcgtgagccttctctccgtgctgctgatgggttgcgtagctgaaaccggtcaattcagcgtgctgggcccttctggacctatcctggctatggttggagaggacgccgacctgccttgccacctgtttccaacaatgagcgccgagacaatggaactgaaatgggtgtccagcagcctgcggcaggtcgtgaatgtgtacgccgatggcaaagaggtggaagatagacagagcgccccttacagaggccggaccagcattctgagagatggcatcacagccggcaaggccgctctgagaatccacaatgtgaccgccagcgacagcggcaagtacctgtgctacttccaagacggcgacttctacgagaaggccctggtcgagctgaaagtggctgccctgggatctgatctgcacgtggacgtgaagggctacaaggatggcggcatccacctggaatgtagaagcaccggctggtatccccagcctcagatccagtggtccaacaacaagggcgagaacatccccaccgtggaagctccagttgtggctgatggcgtgggactgtatgccgtggccgcctctgtgatcatgagaggctctagcggagagggcgtgtcctgcacaatcagatctagcctgctgggcctcgaaaagaccgcctccatctctatcgccgatccattcttcagaagcgcccagtccggaggcctcgaaatcagagccgccttcctgcggagaagaaacacagccctgagaaccagagtggccgagctgagacagagagtgcagcggctgagaaacatcgtgtcccagtacgagacaagatacggccctctcggcggtaccctgaacgatatctttgaggcccagaaaatcgagtggcacgaggactacaaggacgacgacgacaagtgatgactcgag

SEQ ID NO: 37 - protein, 285 aa, synthetic construct
ETGQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSSGGLTPRGVRLGGLEIEAAFLERENTALETRVAELRQRVQRLRNRVSQYRTRYGPLGTKHHHHHH

SEQ ID NO: 38 - protein, 301 aa, Homo sapiens
ETGQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQSGGLTPRGVRLGGLEIRAAFLRQRNTALRTEVAELEQEVQRLENEVSQYETRYGPLGGTLNDIFEAQKIEWHEDYKDDDDK

SEQ ID NO: 39 - DNA, 989 bp, synthetic construct
  source 1..989: mol_type = other DNA
gaattcaagcttgccaccatggggatccttcccagccctgggatgcctgcgctgctctccctcgtgagccttctctccgtgctgctgatgggttgcgtagctgaaaccggtcaattcatcgtcgtgggccccaccgatcctatcctggctacagtgggcgagaacaccacactgagatgccatctgagccccgagaagaacgccgaggatatggaagtgcggtggttcagaagccagttcagccctgccgtgttcgtgtacaaaggcggcagagagcggaccgaggaacagatggaagagtacagaggccggaccaccttcgtgtccaaggacatctctagaggcagcgtggccctggtcatccacaacatcacagcccaagagaacggcacctacaggtgctacttccaagagggcagaagctacgacgaggccatcctgcatctggtggttgccggactgggaagcaagcccctgatctctatgagaggccacgaggacggcggcatcagactggaatgtatcagcagaggctggtatcccaagccactgaccgtttggagagatccctatggcggagtggcccctgctctgaaagaagtgtctatgcccgatgccgacggcctgttcatggtcacaacagccgtgatcatccgggacaagagcgtgcggaatatgagctgcagcatcaacaacaccctgctgggccagaaaaaagagagcgtcatcttcatccccgagtccttcatgcccagcgttagttccggaggcctgacacctagaggtgttagacttggaggcctggaaatcgaggccgccttcctggaaagagagaacacagccctggaaaccagagtggccgagctgagacagagagtgcagcggctgagaaaccgggtgtcccagtacagaaccagatacggccctctgggtaccaagcaccaccaccatcaccactgatgactcgagctcacaattccacacaacat

SEQ ID NO: 40 - DNA, 1037 bp, synthetic construct
  source 1..1037: mol_type = other DNA
gaattcaagcttgccaccatggggatccttcccagccctgggatgcctgcgctgctctccctcgtgagccttctctccgtgctgctgatgggttgcgtagctgaaaccggtcaattcagcgtgctgggcccttctggacctatcctggctatggttggagaggacgccgacctgccttgccacctgtttccaacaatgagcgccgagacaatggaactgaaatgggtgtccagcagcctgcggcaggtcgtgaatgtgtacgccgatggcaaagaggtggaagatagacagagcgccccttacagaggccggaccagcattctgagagatggcatcacagccggcaaggccgctctgagaatccacaatgtgaccgccagcgacagcggcaagtacctgtgctacttccaagacggcgacttctacgagaaggccctggtcgagctgaaagtggctgccctgggatctgatctgcacgtggacgtgaagggctacaaggatggcggcatccacctggaatgtagaagcaccggctggtatccccagcctcagatccagtggtccaacaacaagggcgagaacatccccaccgtggaagctccagttgtggctgatggcgtgggactgtatgccgtggccgcctctgtgatcatgagaggctctagcggagagggcgtgtcctgcacaatcagatctagcctgctgggcctcgaaaagaccgcctccatctctatcgccgatccattcttcagaagcgcccagtccggaggcctgacacctagaggtgttagacttggaggcctggaaatcagagccgccttcctgcggcagagaaacacagccctgagaacagaggtggccgagctggaacaagaggtgcagcggctggaaaacgaggtgtcccagtacgagacaagatacggccctctgggcggtaccctgaacgatatctttgaggcccagaaaatcgagtggcacgaggactacaaggacgacgacgacaagtgatgactcgagctcacaattccacacaacat

SEQ ID NO: 41 - protein, 285 aa, synthetic construct
ETGQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQECRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSSGGLTPRGVRLGGLEIEAAFLEQENTALETEVAELEQEVQRLENIVSQYETRYGPLGTKHHHHHH

SEQ ID NO: 42 - protein, 301 aa, synthetic construct
ETGQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGCFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQSGGLTPRGVRLGGLEIRAAFLRRRNTALRTRVAELRQRVQRLRNIVSQYETRYGPLGGTLNDIFEAQKIEWHEDYKDDDDK

SEQ ID NO: 43 - DNA, 969 bp, synthetic construct
  source 1..969: mol_type = other DNA
gaattcaagcttgccaccatggggatccttcccagccctgggatgcctgcgctgctctccctcgtgagccttctctccgtgctgctgatgggttgcgtagctgaaaccggtcaattcatcgtcgtgggtcccaccgatcctatcctggctacagtgggcgagaacaccacactgagatgccatctgagccccgagaagaacgccgaggatatggaagtgcggtggttcagaagccagttcagccctgccgtgttcgtgtacaaaggcggcagagagcggaccgaggaacagatggaagagtacagaggccggaccaccttcgtgtccaaggacatctctagaggcagcgtggccctggtcatccacaacatcacagcccaagagaacggcacctacaggtgctacttccaagagtgcagaagctacgacgaggccatcctgcatctggtggttgccggactgggaagcaagcccctgatctctatgagaggccacgaggacggcggcatcagactggaatgtatcagcagaggctggtatcccaagccactgaccgtttggagagatccctatggcggagtggcccctgctctgaaagaagtgtctatgcccgatgccgacggcctgttcatggtcacaacagccgtgatcatccgggacaagagcgtgcggaatatgagctgcagcatcaacaacaccctgctgggccagaaaaaagagagcgtcatcttcattccggagtccttcatgcccagcgttagtagcggaggcctgacacctagaggcgttagacttggaggcctggaaatcgaggccgccttcctggaacaagagaacacagccctggaaacagaggtggccgagcttgagcaagaggtgcagcggctggaaaacatcgtgtcccagtacgagacaagatacggccctctgggtaccaagcaccaccaccatcaccactgatgactcgag

SEQ ID NO: 44 - DNA, 1017 bp, synthetic construct
  source 1..1017: mol_type = other DNA
gaattcaagcttgccaccatggggatccttcccagccctgggatgcctgcgctgctctccctcgtgagccttctctccgtgctgctgatgggttgcgtagctgaaaccggtcaattcagcgtgctgggtccttctggacctatcctggctatggttggagaggacgccgacctgccttgccacctgtttccaacaatgagcgccgagacaatggaactgaaatgggtgtccagcagcctgcggcaggtcgtgaatgtgtacgccgatggcaaagaggtggaagatagacagagcgccccttacagaggccggaccagcattctgagagatggcatcacagccggcaaggccgctctgagaatccacaatgtgaccgccagcgacagcggcaagtacctgtgctacttccaagacggctgcttctacgagaaggccctggtcgagctgaaagtggctgccctgggatctgatctgcacgtggacgtgaagggctacaaggatggcggcatccacctggaatgtagaagcaccggctggtatccccagcctcagatccagtggtccaacaacaagggcgagaacatccccaccgtggaagctccagttgtggctgatggcgtgggactgtatgccgtggccgcctctgtgatcatgagaggctctagcggagagggcgtgtcctgcacaatcagatctagcctgctgggcctcgaaaagaccgcctccatctctatcgccgatccattcttccggagcgcccagtcaggaggcctgacacctagaggtgttagacttggaggcctggaaatcagagccgccttcctgcggagaagaaacacagccctgagaaccagagtggccgagctgagacagagagtgcagcggctgagaaacatcgtgtcccagtacgagacaagatacggccctctcggcggtaccctgaacgatatctttgaggcccagaaaatcgagtggcacgaggactacaaggacgacgacgacaagtgatgactcgag

SEQ ID NO: 45 - protein, 285 aa, synthetic construct; identical to SEQ ID NO: 29

SEQ ID NO: 46 - protein, 301 aa, synthetic construct
ETGQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYAKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQSGGLTPRGVRLGGLEIRAAFLRRRNTALRTRVAELRQRVQRLRNIVSQYETRYGPLGGTLNDIFEAQKIEWHEDYKDDDDK

SEQ ID NO: 47 - DNA, 969 bp, synthetic construct; identical to SEQ ID NO: 31
  source 1..969: mol_type = other DNA

SEQ ID NO: 48 - DNA, 1554 bp, synthetic construct
  source 1..1554: mol_type = other DNA
gaattcgccaccatgaagatggccagctttctggccttcctgctgctgaacttcagagtgtgtctgctgctgctccagctgctgatgcctcacagcgcccagttttctgtgctgggcccttctggacctatcctggccatggtgggagaagatgccgacctgccttgccatctgttccctaccatgagcgccgagacaatggaactgaaatgggtgtccagcagcctgcggcaggtcgtgaatgtgtacgccgatggcaaagaggtggaagatagacagagcgccccttacagaggccggaccagcattctgagagatggcatcacagccggcaaggccgctctgagaatccacaatgtgaccgccagcgacagcggcaagtacctgtgctacttccaagacggcgacttctacgcgaaggccctggtcgagctgaaagtggctgccctgggatctgatctgcacgtggacgtgaagggctacaaggatggcggcatccacctggaatgtagaagcacaggatggtacccccagcctcagatccagtggtccaacaacaagggcgagaacatccccaccgtggaagctccagttgtggctgatggcgtgggactgtatgccgtggccgcctctgtgatcatgagaggctctagcggagagggcgtgtcctgcacaatcagatctagcctgctgggcctcgaaaagaccgcctccatctctatcgccgatccattctttcggagcgcccagagatggatcgctgctctggctggaacactgcccgttctgctgttgcttcttggcggagccggctactttctgtggcagcagcaagaggaaaagaaaacccagttccggaagaagaagcgcgagcaagagctgcgcgaaatggcctggtccaccatgaagcaagagcagtccaccagagtgaagctgctggaagaactgcggtggcggagcatccagtatgcatctcggggagagagacattcagcctataatgaatggaaaaaggccctcttcaagcctgcggatgtgattctggatccaaaaacagcaaaccccatcctccttgtttctgaggaccagaggagtgtgcagcgtgccaaggagccccaggatctgccagacaaccctgagagatttaattggcattattgtgttctcggctgtgagagcttcatatcagggagacattactgggaggtggaggtaggggacaggaaagagtggcatataggggtgtgcagtaagaatgtgcagagaaaaggctgggtcaaaatgacacctgagaatggattctggactatggggctgactgatgggaataagtatcggactctaactgagcccagaaccaacctgaaacttcctaagccccctaagaaagtgggggtcttcctggactatgagactggagatatctcattctacaatgctgtggatggatcgcatattcatactttcctggacgtctccttctctgaggctctatatcctgttttcagaattttgaccttggagcccacggccctgactatttgtccagcgtga

SEQ ID NO: 49 - protein, 44 aa, synthetic construct
LEIEAAFLEQENTALETEVAELEQEVQRLENIVSQYETRYGPLG

SEQ ID NO: 50 - protein, 43 aa, synthetic construct
LEIRAAFLRRRNTALRTRVAELRQRVQRLRNIVSQYETRYGPL

SEQ ID NO: 51 - protein, 44 aa, synthetic construct
LEIEAAFLERENTALETRVAELRQRVQRLRNRVSQYRTRYGPLG

SEQ ID NO: 52 - protein, 43 aa, synthetic construct
LEIRAAFLRQRNTALRTEVAELEQEVQRLENEVSQYETRYGPL

SEQ ID NO: 53 - skipped

SEQ ID NO: 54 - protein, 13 aa, synthetic construct
SGGLTPRGVRLGG

SEQ ID NO: 55 - RNA, 20 nt, synthetic construct
  source 1..20: mol_type = other RNA
aactttcacctacaaaccgc

SEQ ID NO: 56 - RNA, 20 nt, synthetic construct
  source 1..20: mol_type = other RNA
gaaccctgactgaaacgatc

SEQ ID NO: 57 - DNA, 21 bp, synthetic construct
  source 1..21: mol_type = other DNA
tctgccatcgcgctggtgcct

SEQ ID NO: 58 - DNA, 19 bp, synthetic construct
  source 1..19: mol_type = other DNA
catcacgccagcccaaaac

SEQ ID NO: 59 - DNA, 23 bp, synthetic construct
  source 1..23: mol_type = other DNA
gcctgaacacgcgaccgtgcctg

SEQ ID NO: 60 - DNA, 18 bp, synthetic construct
  source 1..18: mol_type = other DNA
accagctcgatggcagac

SEQ ID NO: 61 - DNA, 21 bp, synthetic construct
  source 1..21: mol_type = other DNA
cgtgccagccgcactgcggtg

SEQ ID NO: 62 - DNA, 24 bp, synthetic construct
  source 1..24: mol_type = other DNA
ccaatagacacaggcacggtctgg

SEQ ID NO: 63 - DNA, 27 bp, synthetic construct
  source 1..27: mol_type = other DNA
gtgtagcatggcgggcgaagccatcgg

SEQ ID NO: 64 - DNA, 18 bp, synthetic construct
  source 1..18: mol_type = other DNA
cgcagtgtggctggcacg

SEQ ID NO: 65 - DNA, 23 bp, synthetic construct
  source 1..23: mol_type = other DNA
ctacagagaggcggacatctacg

SEQ ID NO: 66 - DNA, 18 bp, synthetic construct
  source 1..18: mol_type = other DNA
atgaaggtcatggtgttg

SEQ ID NO: 67 - DNA, 21 bp, synthetic construct
  source 1..21: mol_type = other DNA
agagagaaggccatctacggc

SEQ ID NO: 68 - DNA, 19 bp, synthetic construct
  source 1..19: mol_type = other DNA
gtagatgaaggtcatggtg

SEQ ID NO: 69 - DNA, 22 bp, synthetic construct
  source 1..22: mol_type = other DNA
ccctggcttcgcggacaacttc

SEQ ID NO: 70 - DNA, 19 bp, synthetic construct
  source 1..19: mol_type = other DNA
ccgtagatgtccttctctc

SEQ ID NO: 71 - DNA, 21 bp, synthetic construct
  source 1..21: mol_type = other DNA
ggcttcaaggccaacttccag

SEQ ID NO: 72 - DNA, 18 bp, synthetic construct
  source 1..18: mol_type = other DNA
agggccgtagatgtcctt

SEQ ID NO: 73 - DNA, 29 bp, synthetic construct
  source 1..29: mol_type = other DNA
ggacaacttcgcgggcgacatcgacattg

SEQ ID NO: 74 - DNA, 18 bp, synthetic construct
  source 1..18: mol_type = other DNA
ttgaagccagggccgtag

SEQ ID NO: 75 - DNA, 21 bp, synthetic construct
  source 1..21: mol_type = other DNA
ggcgacatcgccattgccaag

SEQ ID NO: 76 - DNA, 20 bp, synthetic construct
  source 1..20: mol_type = other DNA
ctggaagttgtccttgaagc

SEQ ID NO: 77 - DNA, 23 bp, synthetic construct
  source 1..23: mol_type = other DNA
cgacatcgacgctgccaagaacc

SEQ ID NO: 78 - DNA, 18 bp, synthetic construct
  source 1..18: mol_type = other DNA
ccctggaagttgtccttg

SEQ ID NO: 79 - DNA, 22 bp, synthetic construct
  source 1..22: mol_type = other DNA
cgacattgccgcgaacctggcc

SEQ ID NO: 80 - DNA, 18 bp, synthetic construct
  source 1..18: mol_type = other DNA
atgtcgccctggaagttg

SEQ ID NO: 81 - DNA, 22 bp, synthetic construct
  source 1..22: mol_type = other DNA
ggccgtgctggcgattctggcc

SEQ ID NO: 82 - DNA, 18 bp, synthetic construct
  source 1..18: mol_type = other DNA
aggttcttggcaatgtcg

SEQ ID NO: 83 - DNA, 25 bp, synthetic construct
  source 1..25: mol_type = other DNA
gctgaagattgcggcccctagcgag

SEQ ID NO: 84 - DNA, 18 bp, synthetic construct
  source 1..18: mol_type = other DNA
acggccaggttcttggca

SEQ ID NO: 85 - DNA, 22 bp, synthetic construct
  source 1..22: mol_type = other DNA
caagggcaccgcagtgaccgtg

SEQ ID NO: 86 - DNA, 18 bp, synthetic construct
  source 1..18: mol_type = other DNA
ccgaagatcagcttgtcg

SEQ ID NO: 87 - DNA, 20 bp, synthetic construct
  source 1..20: mol_type = other DNA
tgcaaaagcactgcagacac

SEQ ID NO: 88 - DNA, 20 bp, synthetic construct
  source 1..20: mol_type = other DNA
agccagcctgagggtaactt

SEQ ID NO: 89 - DNA, 21 bp, synthetic construct
  source 1..21: mol_type = other DNA
tttcttaggattctgcccgcc

SEQ ID NO: 90 - DNA, 20 bp, synthetic construct
  source 1..20: mol_type = other DNA
tccttagggcccaggactat

SEQ ID NO: 91 - DNA, 20 bp, synthetic construct
  source 1..20: mol_type = other DNA
accagaagtaccactggctt

SEQ ID NO: 92 - DNA, 22 bp, synthetic construct
  source 1..22: mol_type = other DNA
aaccctgttttctgccttaaca

SEQ ID NO: 93 - RNA, 20 nt, synthetic construct
  source 1..20: mol_type = other RNA
gactttcatataccgagaaa

SEQ ID NO: 94 - RNA, 20 nt, synthetic construct
  source 1..20: mol_type = other RNA
ggccatagatgtccttttct

SEQ ID NO: 95 - DNA, 83 bp, synthetic construct
  source 1..83: mol_type = other DNA
accttggaaattgtctttgaaaccagggccatagatgtccgcctctcggtatatgaaagtcattgtgttaccttgggtcttcc

SEQ ID NO: 96 - protein, 116 aa, synthetic construct
QFIVVGPTDPILATVGENTTLRCHLSPEKNAEDMEVRWFRSQFRPAVFVYKGGRERTEEQMEEYRGRTTFVSKDISRGSVALVIHNITAQENGTYRCYFQEGRSYDEAILHLVVAG

SEQ ID NO: 97 - protein, 499 aa, synthetic construct
QFIVVGPTDPILATVGENTTLRCHLSPEKNAEDMEVRWFRSQFRPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 98 - DNA, 348 bp, synthetic construct
  source 1..348: mol_type = other DNA
caattcatcgtcgtgggccccaccgatcctatcctggctacagtgggcgagaacaccacactgagatgccatctgagccccgagaagaacgccgaggatatggaagtgcggtggttcagaagccagttcagacctgccgtgttcgtgtacaaaggcggcagagagcggaccgaggaacagatggaagagtacagaggccggaccaccttcgtgtccaaggacatctctagaggcagcgtggccctggtcatccacaacatcacagcccaagagaacggcacctacaggtgctacttccaagagggcagaagctacgacgaggccatcctgcatctggtggttgccgga

SEQ ID NO: 99 - DNA, 1503 bp, synthetic construct
  source 1..1503: mol_type = other DNA
cagtttattgtcgtgggcccgaccgatcctattctcgctacagtgggcgaaaatacaacacttaggtgtcatttgtctccggagaaaaacgctgaggatatggaggtaaggtggtttagaagtcaattcagaccggctgttttcgtttataaaggcggtagagaacgaactgaagaacaaatggaagagtatcgaggacgcacaacgttcgttagcaaagacattagcaggggctctgtggcgttggttatccacaacattactgcccaagagaatggtacatataggtgttactttcaagaaggaaggtcatatgatgaggcaattcttcatttggtggttgcgggactgggctccaaaccgctgatctccatgagaggccatgaggacgggggtatccgactggagtgcatatctcggggctggtatcccaagccactcacagtctggagggatccctacggtggggtcgccccagcattgaaggaagtttctatgccagacgccgacggcctgtttatggttaccaccgcggtgattatacgcgacaaatcagtacgcaatatgagttgctccatcaataatactctgctcggtcagaaaaaggagagtgtcatctttatcccagaatctttcatgcccagcgtaagcccatgtgctgtagcgctgcctatcatagtagtaatcctgatgattcccatagcagtctgtatttactggataaataagttgcagaaagaaaagaaaatactctctggggagaaggagttcgagcgcgaaaccagagagatcgctcttaaggaattggaaaaggaaagggtgcagaaggaggaagaactccaggtaaaggagaaattgcaagaagaactccggtggcgccgcacatttctccatgccgtcgatgtagtcctggacccagacactgcgcacccagacctgttcttgtccgaggatcggcgcagtgtcaggaggtgcccgttccggcatttgggtgaatctgtcccagacaatccggaaaggtttgacagtcagccgtgcgtcctcggcagagaatcatttgcttctggaaagcactactgggaggttgaagtcgagaacgtaattgaatggacggtgggtgtgtgccgggacagtgtcgagcggaaaggagaagttctgcttatcccgcagaatggattttggacgcttgaaatgcacaaaggccaatatagagccgtaagttctcccgaccgaattttgccccttaaagagagcttgtgtagagtaggtgtgttcctcgactacgaggctggcgatgtctcattctataatatgcgggataggtctcacatatacacctgtccgcgctccgcattcagtgtcccagtgcgaccattttttcgcttgggatgtgaagatagcccgatattcatatgcccagcattgactggtgccaatggcgtcaccgtcccggaagaaggtctgacccttcatagagttgggacccatcaatccctttaatga

(19) United States
(12) Patent Application Publication
(10) Pub. No.: US 2025/0179144 A1
(43) Pub. Date: Jun. 5, 2025

(54) Modified Butyrophilin and Butyrophilin Complexes

(71) Applicant: The University of Melbourne, Melbourne (AU)

(72) Inventors: Adam Peter Uldrich, Melbourne (AU); Thomas Samuel Fulford, Melbourne (AU); Nicholas Anthony Gherardin, Melbourne (AU); Marc Rigau Cortal, Melbourne (AU); Dale Ian Godfrey, Melbourne (AU)

(21) Appl. No.: 18/717,911

(22) PCT Filed: Dec. 9, 2022
(86) PCT No.: PCT/AU2022/051485

§ 371 Date: Jun. 7, 2024

(30) Foreign Application Priority Data
Dec. 9, 2021 (AU) 2021903993

(51) Int. Cl. C07K 14/705; A61K 38/17; C12N 15/62; C12N 15/85

(52) U.S. Cl. CPC C07K 14/70503; A61K 38/1774; C12N 15/62; C12N 15/85; C07K 2319/30; C07K 2319/735

(57) ABSTRACT

The present disclosure relates to modified butyrophilin 2A and 3A, and butyrophilin 2A-3A complexes and use thereof to induce or enhance TCR activation.

### TECHNICAL FIELD

The present disclosure relates to modified butyrophilin 2A and 3A, and butyrophilin 2A-3A complexes and methods of use.

### INCORPORATION BY REFERENCE OF SEQUENCE LISTING PROVIDED AS A SEQUENCE LISTING XML FILE

A Sequence Listing is provided herewith as a Sequence Listing XML, “PHIL-004_SEQ_LIST”, created on Feb. 3, 2025 and having a size of 122,385 bytes. The contents of the Sequence Listing XML are incorporated herein by reference in their entirety.

### BACKGROUND OF INVENTION

Most alpha-beta (αβ) T cells become activated following recognition of peptide fragments in complex with major histocompatibility complex molecules (pMHC), which are sensed by somatically rearranged T cell receptors (αβTCRs) in a one-receptor one-ligand fashion. By contrast, gamma-delta (γδ) T cells represent a separate lineage of MHC-unrestricted T cells that express rearranged antigen (Ag) receptors derived from the TCRγ (TRG) and TCRδ (TRD) gene loci. These cells play a key role in the priming and effector phases of immunity to infectious diseases as well as in tissue surveillance.

In humans, the majority of circulating γδ T cells express a semi-invariant Vγ9Vδ2+(TRGV9-TRGV2) γδbTCR that confers reactivity to a distinct class of non-peptide Ag, termed phosphoantigens (pAgs), which are metabolic intermediates in the biosynthesis of isoprenoids. There are two classes of pAg; those derived from the non-mevalonate pathway such as 4-hydroxy-3-methylbut-2-enyl pyrophosphate (HMBPP), found in bacteria and apicomplexan parasites, and those derived from the mevalonate pathway such, as isopentenyl pyrophosphate (IPP), found in vertebrates. Both foreign and self-pAg are stimulatory for γδ T cells to differing degrees, and facilitate potent anti-microbial and anti-tumor immunity, respectively.

Butyrophilin (BTN) and butyrophilin-like (BTNL) molecules are a family of surface expressed transmembrane proteins that are typically comprised of extracellular immunoglobulin-superfamily variable (IgV)- and constant (IgC)-like domains, as well as an intracellular B30.2 domain. In certain combinatorial pairs, BTN and BTNL molecules support the activation of discrete γδ T cell subsets. For instance, BTNL3 and BTNL8 are expressed by gut epithelia and cooperate to facilitate the activation of Vγ4+γδ T cells. Likewise, in mice, Btnl1 and Btnl6 facilitate the activation of gut-resident Vγ7+74 γδ T cells, and the Btnl family members Skint1 and Skint2 are important for the development and function of skin resident Vγ5Vδ1+ dendritic epidermal T cells (DETCs).

In humans, BTN member 3A1 (BTN3A1) sequesters pAg via a positively charged pocket within its intracellular B30.2 domain, which is an essential step in the initiation of Vγ9Vδ2+ T cell activation. Together with BTN member 2A1 (BTN2A1), BTN2A1 and BTN3A1 mediate γδ T cell responses to pAg. Thus, BTN molecules have emerged as important regulators of γδ T cell-mediated immunity and do so as heteromeric pairs.

There is however still a need to better understand the mechanisms that govern pAg recognition to provide novel immunotherapies and agents that can induce γδ T cell responses in, for example, cancer patients, or patients with chronic infections, or patients with autoimmune or inflammatory diseases.

### SUMMARY OF INVENTION

The inventors have surprisingly demonstrated that the BTN3 IgV domain interacts with BTN2 and that a BTN2-BTN3 complex and a modified BTN3 can react with Vγ9Vδ2⁺ TCR, facilitating γδ T cell-mediated immunity. The inventors have also surprisingly demonstrated a modified BTN2 can enhance the ability of the BTN2-BTN3 complex to react with Vγ9Vδ2⁺ TCR.

Accordingly, the present disclosure provides a recombinant BTN2-BTN3 heteromeric complex, wherein the BTN2-BTN3 heteromeric complex binds to a Vδ2⁺TCR (e.g., Vγ9Vδ2⁺ TCR). In some embodiments, the BTN2-BTN3 heteromeric complex induces or enhances Vδ2⁺ TCR activation. Any means of complexing BTN2 and BTN3 (e.g., means of forming a heterodimerisation domain) known in the art may be used.

In some embodiments, the BTN2-BTN3 heteromeric complex of the invention comprises two polypeptide chains, wherein the first polypeptide comprises an IgV-like domain of BTN2 or a Vγ9+ TCR binding portion thereof and the second polypeptide comprises an IgV-like domain of BTN3 or a Vδ2+ TCR binding portion thereof and a linker. In some embodiments, the heteromeric protein comprises two individual polypeptide chains which self-associate. In other embodiments, the heteromeric protein comprises two individual polypeptide chains that are induced to associate.

In one embodiment, the BTN2-BTN3 heteromeric complex comprises:
(i) a first polypeptide comprising an IgV-like domain of BTN2 or a Vγ9+ TCR binding portion thereof;
(ii) a second polypeptide comprising an IgV-like domain of BTN3 or a Vδ2+ TCR binding portion thereof and a second heterologous C-terminal dimerization peptide,

wherein the first and second polypeptides wherein the BTN2-BTN3 heteromeric complex binds to a Vγ9Vδ2⁺ TCR.

In another embodiment, the BTN2-BTN3 heteromeric complex comprises:
(i) a first polypeptide comprising an IgV-like domain of BTN2 or a Vγ9+ TCR binding portion thereof and a first heterologous C-terminal dimerization peptide,
(ii) a second polypeptide comprising an IgV-like domain of BTN3 or a Vδ2+ TCR binding portion thereof and a second heterologous C-terminal dimerization peptide which specifically dimerizes with the first heterologous C-terminal dimerization peptide to form a heterodimerization domain,

wherein the BTN2-BTN3 heteromeric complex binds to a Vγ9Vδ2⁺ TCR.

In some embodiments, the heterodimerization domain comprises a charge polarized core domain. For example, the first and second heterologous C-terminal dimerization peptides comprise positively or negatively charged amino acid residues. In another example, the first and second heterologous C-terminal dimerization peptides comprise polar residues which can H-bond, or non-polar residues which can form van der waals contacts.

In one embodiment, the first heterologous C-terminal dimerization peptide comprises positively charged amino acids, optionally joined by a linker to the first polypeptide and the second heterologous C-terminal dimerization peptide comprises negatively charged amino acids, optionally joined by a linker to the second polypeptide.

In another embodiment, the first heterologous C-terminal dimerization peptide comprises negatively charged amino acids, optionally joined by a linker to the first polypeptide and the second heterologous C-terminal dimerization peptide comprises positively charged amino acids, optionally joined by a linker to the second polypeptide.

In some embodiments, formation of the BTN2-BTN3 heteromeric complex is driven by electrostatic interactions between the positively charged and negatively charged amino acid residues in the first and second dimerization peptides.

Further, formation of homodimeric proteins is inhibited by the repulsion between the positively charged or negatively charged amino acid residues in the first or second dimerization peptides.

In some embodiments, the dimerization peptide is about 2 to about 50 amino acids long. For example, the dimerization peptide may be about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In some embodiments, the dimerization peptide may include one or more positively charged amino acids selected from His, Lys, and Arg. In other embodiments, the dimerization peptide may include one or more negatively charged amino acids selected from Asp and Glu.

In some embodiments the first and second dimerization peptides have reduced affinity for themselves.

In one embodiment, the heterodimerisation domain is a coiled coil domain. For example, the dimerisation peptides are acidic and basic zippers, for example fos and jun dimerization peptides such as c-jun and c-fos dimerisation peptides.

In some embodiment the first or second dimerization peptide comprises the amino acid sequence shown in SEQ ID NO:49, 50, 51, 52 or an amino acid sequence having at least 90%, or 93%, or 95%, or 97%, or 98%, or 99% identity thereto.

In some embodiments, the dimerization peptides are linked to the C-terminal end of the BTN2 and BTN3 optionally via a linker comprising spacer amino acids such as serine or glycine. The linker may be a short C-terminal linker (e.g., 3-5 residues in length such as SGG; SEQ ID NO:49) or a long C-terminal linker (e.g. 5-15 residues in length). The linker may comprise a cleavage site to for example, allow for cleavage of the heterodimerisation domain from the BTN2-BTN3 heteromeric complex (e.g., SGGLTPRGVRLGG; SEQ ID NO:50).

In one or a further embodiment, the heterodimerisation domain comprises a disulphide bond. For example, the dimerisation peptides comprise one or more Cystein (Cys) residues. In some embodiments, the dimerisation peptides comprise one or more Cys residues to facilitate disulfide bonding between the electrostatically charged amino acids as an additional method to stabilize the BTN2-BTN3 heteromeric complex.

In one embodiment, the heterodimerisation domain comprises an Fc domain of an antibody (e.g., of IgG, IgA, IgD, and IgE, inclusive of subclasses (e.g., IgGI, IgG2, IgG3, and IgG4, and IgAI and IgA2)). In some embodiments, the dimerization peptides comprise Fc derived binding pair. For example, a hinge-CH2-CH3 Fc binding pair derived from, for example, a human IgG1 or IgG4 antibody.

In some embodiments, the Fc domain exhibits increased affinity for and enhanced binding to the neonatal Fc receptor (FcRn). In some embodiments, the Fc domain includes one or more mutations that increases the affinity and enhances binding to FcRn. Without wishing to be bound by theory, it is believed that increased affinity and enhanced binding to FcRn increases the in vivo half-life of the present heteromeric complexes.

In some embodiments, the Fc domain contains one or more amino acid substitutions at amino acid residue 250, 252, 254, 256, 308, 309, 311, 428, 433 or 434 (in accordance with Kabat numbering), or equivalents thereof.

In one embodiment, the amino acid substitution at amino acid residue 250 is a substitution with glutamine.

In one embodiment, the amino acid substitution at amino acid residue 252 is a substitution with tyrosine, phenylalanine, tryptophan or threonine.

In one embodiment, the amino acid substitution at amino acid residue 254 is a substitution with threonine.

In one embodiment, the amino acid substitution at amino acid residue 256 is a substitution with serine, arginine, glutamine, glutamic acid, aspartic acid, or threonine.

In one embodiment, the amino acid substitution at amino acid residue 308 is a substitution with threonine.

In one embodiment, the amino acid substitution at amino acid residue 309 is a substitution with proline.

In one embodiment, the amino acid substitution at amino acid residue 311 is a substitution with serine.

In one embodiment, the amino acid substitution at amino acid residue 385 is a substitution with arginine, aspartic acid, serine, threonine, histidine, lysine, alanine or glycine.

In one embodiment, the amino acid substitution at amino acid residue 386 is a substitution with threonine, proline, aspartic acid, serine, lysine, arginine, isoleucine, or methionine.

In one embodiment, the amino acid substitution at amino acid residue 387 is a substitution with arginine, proline, histidine, serine, threonine, or alanine.

In one embodiment, the amino acid substitution at amino acid residue 389 is a substitution with proline, serine or asparagine.

In one embodiment, the amino acid substitution at amino acid residue 428 is a substitution with leucine.

In one embodiment, the amino acid substitution at amino acid residue 433 is a substitution with arginine, serine, isoleucine, proline, or glutamine.

In one embodiment, the amino acid substitution at amino acid residue 434 is a substitution with histidine, phenylalanine, or tyrosine.

In some embodiments, the Fc domain (e.g., comprising an IgG constant region) comprises one or more mutations such as substitutions at amino acid residue 252, 254, 256, 433, 434, or 436 (in accordance with Kabat numbering).

In one embodiment, the IgG constant region includes a triple M252Y/S254T/T256E mutation or YTE mutation.

In another embodiment, the IgG constant region includes a triple H433K/N434F/Y436H mutation or KFH mutation.

In a further embodiment, the IgG constant region includes an YTE and KFH mutation in combination.

In another embodiment, the BTN2-BTN3 heteromeric complex comprises:
(i) a first polypeptide comprising an IgV-like domain of BTN2 or a Vγ9+ TCR binding portion thereof,
(ii) a second polypeptide comprising an IgV-like domain of BTN3 or a Vδ2+ TCR binding portion thereof, and
(iii) a linker linking said first and second polypeptides into a single chain polypeptide,

wherein the BTN2-BTN3 heteromeric complex binds to a Vγ9Vδ2+ TCR.

In some embodiments, the first polypeptide is at the N-terminus and the second polypeptide is at the C-terminus of the polypeptide chain. In other embodiments, the first polypeptide is at the C-terminus and the second polypeptide is at the N-terminus of the polypeptide chain.

In some embodiments, the linker is a peptide or polypeptide linker. In some embodiments, the linker is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In one embodiment, the linker comprises from about 50 to 150 amino acids, for example from about 100 to 150 amino acids.

The linker may be flexible or rigid.

In various embodiments, the linker is substantially comprised of Glycine (Gly) and Serine (Ser) residues (e.g., about 30%, or about 40%, or about 50%, or about 60%, or about 70%, or about 80%, or about 90%, or about 95%, or about 97% Gly and Ser. A Gly/Ser rich linker is typically flexible.

In one embodiment, the linker does not comprise any Proline (Pro) residues.

In other embodiments, the linker is not substantially comprised of glycine and serine residues and may be rigid. In some embodiments, the linker is a hinge region of an antibody (e.g., of IgG, IgA, IgD, and IgE, inclusive of subclasses (e.g., IgGI, IgG2, IgG3, and IgG4, and IgA1 and IgA2)). The hinge region, found in IgG, IgA, IgD, and IgE class antibodies, typically acts as a flexible spacer.

In some embodiments, the linker comprises an Fc domain of an antibody (e.g., of IgG, IgA, IgD, and IgE, inclusive of subclasses (e.g., IgGI, IgG2, IgG3, and IgG4, and IgAI and IgA2)). In various embodiments, the linker comprises a hinge-CH2-CH3 Fc domain derived from a human IgG4 antibody. In some embodiments, the linker comprises a hinge-CH2-CH3 Fc domain derived from a human IgGI antibody. In some embodiments, the Fc domain exhibits increased affinity for and enhanced binding to the neonatal Fc receptor (FcRn). In some embodiments, the Fc domain includes one or more mutations that increases the affinity and enhances binding to FcRn. Without wishing to be bound by theory, it is believed that increased affinity and enhanced binding to FcRn increases the in vivo half-life of the present heterodimeric proteins.

In some embodiments, the Fc domain contains one or more amino acid substitutions at amino acid residue 250, 252, 254, 256, 308, 309, 311, 428, 433 or 434 (in accordance with Kabat numbering), or equivalents thereof.

In one embodiment, the amino acid substitution at amino acid residue 250 is a substitution with glutamine.

In one embodiment, the amino acid substitution at amino acid residue 252 is a substitution with tyrosine, phenylalanine, tryptophan or threonine.

In one embodiment, the amino acid substitution at amino acid residue 254 is a substitution with threonine.

In one embodiment, the amino acid substitution at amino acid residue 256 is a substitution with serine, arginine, glutamine, glutamic acid, aspartic acid, or threonine.

In one embodiment, the amino acid substitution at amino acid residue 308 is a substitution with threonine.

In one embodiment, the amino acid substitution at amino acid residue 309 is a substitution with proline.

In one embodiment, the amino acid substitution at amino acid residue 311 is a substitution with serine.

In one embodiment, the amino acid substitution at amino acid residue 385 is a substitution with arginine, aspartic acid, serine, threonine, histidine, lysine, alanine or glycine.

In one embodiment, the amino acid substitution at amino acid residue 386 is a substitution with threonine, proline, aspartic acid, serine, lysine, arginine, isoleucine, or methionine.

In one embodiment, the amino acid substitution at amino acid residue 387 is a substitution with arginine, proline, histidine, serine, threonine, or alanine.

In one embodiment, the amino acid substitution at amino acid residue 389 is a substitution with proline, serine or asparagine.

In one embodiment, the amino acid substitution at amino acid residue 428 is a substitution with leucine.

In one embodiment, the amino acid substitution at amino acid residue 433 is a substitution with arginine, serine, isoleucine, proline, or glutamine.

In one embodiment, the amino acid substitution at amino acid residue 434 is a substitution with histidine, phenylalanine, or tyrosine.

In some embodiments, the Fc domain (e.g., comprising an IgG constant region) comprises one or more mutations such as substitutions at amino acid residue 252, 254, 256, 433, 434, or 436 (in accordance with Kabat numbering).

In one embodiment, the IgG constant region includes a triple M252Y/S254T/T256E mutation or YTE mutation.

In another embodiment, the IgG constant region includes a triple H433K/N434F/Y436H mutation or KFH mutation.

In a further embodiment, the IgG constant region includes an YTE and KFH mutation in combination.

Dimerization of the BTN2 and BTN3 may induce a conformational change that that switches the BTN molecules from an inactive ‘cryptic’ state into an active ‘open-altered’ state that allows the BTN2-BTN3 complexes to react with the Vγ9Vδ2⁺ TCR, facilitating γδ T cell-mediated immunity. Dimerization of BTN2 and BTN3 may occur concurrently with homodimerization of BTN2 and/or BTN3, such that the BTN2-BTN3 complex consists of an association between BTN2 homodimer paired with BTN3 homodimer, or an association between BTN2 monomer and BTN3 homodimer, or BTN2 dimer with BTN3 monomer, or BTN2 monomer with BTN3 monomer.

In some embodiments, the BTN2 and BTN3 are not in cis conformation. In other or further embodiments, the BTN2 and BTN3 do not interact via the CFG faces of their IgV domains. In other or further embodiments, the BTN2 and BTN3 do not form W-shaped heteromers.

In some embodiments, the BTN2-BTN3 heteromeric complex binds the Vγ9Vδ2⁺ TCR via two spatially distinct epitopes. For example, BTN2 engages the side of Vγ9, and BTN3 binds to the apical surface.

In some embodiments, BTN2 binds the binds the Vγ9+ chain of the TCR via the CFG face of its IgV domain.

In some embodiments, one or more of the CC′-loop, F- and G-strands of the BTN2 bind the Vγ9Vδ2⁺ TCR, for example binds the Vγ9⁺ chain, for example, the B-, D- and E-strands of Vγ9. In some embodiments, the CC′-loop of the BTN2 comprises one or more of amino acids corresponding to Ser41, Gln42, Phe43 and Ser44 of the BTN2 polypeptide sequence shown in SEQ ID NO:1. In some embodiments, one or more of these residues bind the Vγ9Vδ2⁺ TCR, for example bind the Vγ9⁺ chain. In some embodiments these residues are important for Vγ9⁺ T cell activation.

In some or further embodiments, BTN2 binds the side of the γ-chain, binding to the Vγ9-encoded IgV domain. For example, the BTN2 binding site on Vγ9 is distal to both the CDR and HV4 loops.

In some or further embodiments, the BTN2 comprises one or more of amino acids corresponding to Phe43, Tyr98 and Tyr105, and optionally one or more of amino acids corresponding to Ser41, Gln42, Ser44 of the BTN2 polypeptide sequence shown in SEQ ID NO:1.

In some or further embodiments, the BTN2 comprises one or more of amino acids corresponding to Tyr105, Asp106 and Glu107, and optionally one or more of amino acids corresponding to Ser41, Gln42, Phe43, Ser44 of the BTN2 polypeptide sequence shown in SEQ ID NO:1.

In some embodiments one or more of these residues bind the Vγ9Vδ2⁺ TCR, for example bind the Vγ9⁺ chain. In some embodiments these residues are important for Vγ9⁺ T cell activation.

In some or further embodiments, the BTN2 comprises one or more of amino acids corresponding to amino acids Phe43, Gln100, Tyr105 of the BTN2 polypeptide sequence shown in SEQ ID NO:1. For example, the aromatic side chain of Phe43 may sit planar to the guanidinium moiety of the Arg20γ side chain, facilitating a cation-π interaction with a predicted electrostatic binding energy of −4.6 kcal/mol. Arg20γ may also formed a water-mediated H-bond with Gln100 of BTN2A1, along with main chain-mediated H-bonds to the Tyr105 side chain hydroxyl group, providing a structural basis for the importance of Arg20γ in BTN2A1-binding and pAg reactivity.

In some or further embodiments, the BTN2 comprises one or more of amino acids corresponding to amino acids Phe43, Ser44, Gln42, Phe43, Tyr105, Asp106, Glu107 and Arg96 of the BTN2 polypeptide sequence shown in SEQ ID NO:1. For example, Glu70γ and His85γ connected by an intrachain H-bond may bind BTN2A1, with Glu70γ H-bonding to the Phe43 and Ser44 main chains, and His85γ making Van der Waal (VDW) contacts with Ser41, Gln42 and Phe43 on BTN2A1. Further contacts may be made by Lys13γ within the A-strand of Vγ9, which H-bonds to Tyr105, and Lys17γ within the B-strand of Vγ9 forming a salt bridge with Asp106. The adjacent Thr18γ may H-bond with Glu107, and Ser16γ H-bonded to the Arg96 side chain.

In some or further embodiments, the BTN2 comprises an intracellular domain. In some embodiments, the intracellular domain (e.g., in the C-terminal cytoplasmic tail) comprises one or both of amino acids corresponding to amino acids Thr482 and Leu488 of the BTN2 polypeptide sequence shown in SEQ ID NO:9 In some or further embodiments, the intracellular domain (e.g., in the B30.2 domain) comprises an amino acid corresponding to amino acid Arg449 of the BTN2 polypeptide sequence shown in SEQ ID NO:9. In some embodiments these residues are important for Vγ9⁺ T cell activation.

In some embodiments, BTN3 binds the Vγ9Vδ2⁺ TCR, for example binds the Vδ2⁺ chain, via the CFG face of its IgV domain.

In some or further embodiments, the BTN3 comprises one or more of amino acids corresponding to Val39, Arg44, His85, Tyr98, Phe104 and Tyr105, and optionally one or more of amino acids corresponding to Phe26, Lys37, Ser42 and Leu96 of the BTN3 polypeptide sequence shown in SEQ ID NO:2. In some embodiments one or more of these residues bind the Vγ9Vδ2⁺ TCR, for example bind the Vδ2⁺ chain. In some embodiments these residues are important for Vδ2⁺ T cell activation.

In one embodiment the BTN2-BTN3 heteromeric complex binds to a Vγ9Vδ2+ TCR independent of phosphoantigen.

In one embodiment, the BTN2-BTN3 heteromeric complex binds to a Vγ9Vδ2⁺ TCR with enhanced binding compared to BTN2 or BTN3 alone.

In some embodiments, the BTN2-BTN3 heteromeric complex induces or enhances Vγ9Vδ2⁺ TCR activation.

In some embodiments, the BTN2-BTN3 heteromeric complex may comprise an amino acid sequence encoding BTN2 and BTN3 having one or more amino acid modifications relative to any of the known protein sequences. In some embodiments, the one or more amino acid modifications may be independently selected from substitutions, insertions, deletions, and truncations. In some embodiments, the amino acid mutations are amino acid substitutions, and may include conservative and/or non-conservative substitutions. For example, amino acid substitutions that desirably or advantageously alter properties of the BTN chain(s) or complex can be made. In one embodiment, modifications that prevent degradation of the BTN chain(s) or BTN can be made.

In some embodiments, the first polypeptide comprises an amino acid sequence that is at least 70% identical to the sequence shown in SEQ ID NO:1. For example, the first polypeptide comprises the amino acid sequence shown in SEQ ID NO:96.

In some or further embodiments, the second polypeptide comprises an amino acid sequence that is at least 70% identical to the sequence shown in SEQ ID NO:2. For example, the second polypeptide comprises the amino acid sequence shown in SEQ ID NO:4.

In some embodiments, the first polypeptide comprises the amino acid sequence shown in SEQ ID NO:96 and the second polypeptide comprises the amino acid sequence shown in SEQ ID NO:4.

In other embodiments, the first polypeptide comprises an amino acid sequence that is at least 70% identical to the sequence shown in SEQ ID NO:9.

In some or further embodiments, the second polypeptide comprises an amino acid sequence that is at least 70% identical to the sequence shown in SEQ ID NO:15.

In other embodiments, the first polypeptide comprises an amino acid sequence that is at least 70% identical to the sequence shown in SEQ ID NO:29.

In some or further embodiments, the second polypeptide comprises an amino acid sequence that is at least 70% identical to the sequence shown in SEQ ID NO:30.

In other embodiments, the first polypeptide comprises an amino acid sequence that is at least 70% identical to the sequence shown in SEQ ID NO:33.

In some or further embodiments, the second polypeptide comprises an amino acid sequence that is at least 70% identical to the sequence shown in SEQ ID NO:34.

In other embodiments, the first polypeptide comprises an amino acid sequence that is at least 70% identical to the sequence shown in SEQ ID NO:37.

In some or further embodiments, the second polypeptide comprises an amino acid sequence that is at least 70% identical to the sequence shown in SEQ ID NO:38.

In other embodiments, the first polypeptide comprises an amino acid sequence that is at least 70% identical to the sequence shown in SEQ ID NO:45.

In some or further embodiments, the second polypeptide comprises an amino acid sequence that is at least 70% identical to the sequence shown in SEQ ID NO:46.

In one or a further embodiment, the first or second polypeptide comprise one or more Cys residues. In one embodiment, the one or more Cys residues are not incorporated into the IgV-like domain of the BTN2 and/or BTN3. For example, the Cys residue is added C-terminal to the IgV-like domain. In some embodiments, the BTN and/or BTN3 comprise a C-terminal Ig-C-like domain and the one or more Cys resides is added within this domain or C terminal to this domain.

In one embodiment, the BTN2-BTN3 heteromeric complex is soluble. In some embodiments, the first and second polypeptides lack a functional transmembrane domain and a cytoplasmic domain.

In some embodiments, one or both the first and second polypeptides comprise one or modified amino acid residues selected from the group consisting of: a glycosylated amino acid, a PEGylated amino acid, a farnesylated amino acid, an acetylated amino acid, a biotinylated amino acid, and an amino acid conjugated to a lipid moiety.

In one embodiment, one or both the first and second polypeptides, further comprise one or more purification sequences optionally selected from the group consisting of: an epitope tag, a FLAG tag, a polyhistidine sequence, and a GDT fusion.

In one embodiment, one or both the first and second polypeptides is glycosylated and has a glycosylation pattern obtainable from a Expi293 cells cell line.

In one embodiment, the BTN2-BTN3 heteromeric complex binds to activin, for example activin A.

The present disclosure of the also provides a multivalent BTN2-BTN3 complex comprising two or more linked BTN2-BTN3 heteromeric complexes of the disclosure. In some embodiments, the multimers do not form W shaped multimers. In some embodiments, the multimers form M shaped multimers.

The present disclosure also provides a modified BTN3 or Vδ2+ TCR binding fragment thereof, wherein the modified BTN3 comprises an IgV-like domain, wherein the IgV-like domain comprises a modification at a position that corresponds to glutamic acid (E) 106 of the amino acid sequence shown in SEQ ID NO:2.

The modification may be an amino acid substitution, insertion, deletion, or truncation.

In one embodiment, the modification is a glutamic acid (E) to alanine (A), glutamic acid (E) to arginine (R), glutamic acid (E) to aspartic acid (D), glutamic acid (E) to asparagine (N), glutamic acid (E) to cysteine (C), glutamic acid (E) to glutamine (Q), glutamic acid (E) to lysine (K), glutamic acid (E) to glycine (G), glutamic acid (E) to histidine (H), glutamic acid (E) to isoleucine (I), glutamic acid (E) to leucine (L), glutamic acid (E) to methionine (M), glutamic acid (E) to ornithine, glutamic acid (E) to phenylalanine (F), glutamic acid (E) to serine (S), glutamic acid (E) to threonine (T), glutamic acid (E) to tryptophan (W), glutamic acid (E) to tyrosine (Y), or glutamic acid (E) to valine (V), glutamic acid (E) to proline (P), glutamic acid (E) to α-Aminobutyric acid (Abu), glutamic acid (E) to norleucine (Nle), glutamic acid (E) to norvaline (Nva), or artificial amino acid substitution.

In another embodiment, the modification is a glutamic acid (E) to alanine (A), glutamic acid (E) to glycine (G), glutamic acid (E) to isoleucine (I), glutamic acid (E) to leucine (L), glutamic acid (E) to methionine (M), glutamic acid (E) to phenylalanine (F), glutamic acid (E) to proline (P), glutamic acid (E) to tryptophan (W), or glutamic acid (E) to valine (V), or artificial amino acid substitution.

In one embodiment, the modification is a glutamic acid (E) to alanine (A) substitution at position 106.

In one embodiment, the IgV-like domain comprises an amino acid sequence having at least 70% identity to SEQ ID NO: 2.

In some embodiments, the modified BTN3 binds to a Vδ2+ TCR independent of phosphoantigen.

In some embodiments, the modified BTN3 binds to a Vδ2+ TCR with enhanced binding. For example, the modification enhances binding to the TCR by at least about 1.5 fold, at least about 1.6 fold, at least about 1.7 fold, at least about 1.8 fold, at least about 1.9 fold, at least about 2 fold, at least about 2.1 fold, at least about 2.2 fold, at least about 2.3 fold, at least about 2.4 fold, at least about 2.5 fold, preferably, at least about 2 fold, compared to binding of a BTN3 that does not comprise the modification.

In some or further embodiments, the modified BTN3 induces or enhances Vδ2⁺ TCR activation.

The present disclosure also provides a modified BTN2 or Vγ9+ TCR binding fragment thereof, wherein the modified BTN2 comprises an IgV-like domain, wherein the IgV-like domain comprises a modification at a position that corresponds to serine (S) 44 of the amino acid sequence shown in SEQ ID NO:1.

The modification may be an amino acid substitution, insertion, deletion, or truncation.

In one embodiment, the modification is a serine (S) to alanine (A), serine (S) to arginine (R), serine (S) to aspartic acid (D), serine (S) to asparagine (N), serine (S) to cysteine (C), serine (S) to glutamine (Q), serine (S) to lysine (K), serine (S) to glycine (G), serine (S) to histidine (H), serine (S) to isoleucine (I), serine (S) to leucine (L), serine (S) to methionine (M), serine (S) to ornithine, serine (S) to phenylalanine (F), serine (S) to glutamic acid (E), serine (S) to threonine (T), serine (S) to tryptophan (W), serine (S) to tyrosine (Y), or serine (S) to valine (V), serine (S) to proline (P), serine (S) to α-Aminobutyric acid (Abu), serine (S) to norleucine (Nle), serine (S) to norvaline (Nva), or artificial amino acid substitution.

In one embodiment, the modification is a serine (S) to arginine (R) substitution at position 44.

In one embodiment, the IgV-like domain comprises an amino acid sequence having at least 70% identity to SEQ ID NO: 1.

In some embodiments, the modified BTN2 binds to a Vγ9+ TCR independent of phosphoantigen.

In some embodiments, the modified BTN2 binds to a Vγ9+ TCR with enhanced binding, for example, when complexed with a BTN3 (such as a modified BTN3 of the disclosure). For example, the modification enhances binding to the TCR by at least about 1.5 fold, at least about 1.6 fold, at least about 1.7 fold, at least about 1.8 fold, at least about 1.9 fold, at least about 2 fold, at least about 2.1 fold, at least about 2.2 fold, at least about 2.3 fold, at least about 2.4 fold, at least about 2.5 fold, at least about 3 fold, preferably, at least about 2 fold, more preferably, at least about 3 fold, compared to binding of a BTN2 (or BTN2-BTN3 complex) that does not comprise the BTN2 Ser44 modification.

In some or further embodiments, the modified BTN2 induces or enhances Vγ9+ TCR activation when for example, complexed with BTN3 (such as a modified BTN3 of the disclosure).

The present disclosure also provides one or more nucleic acids encoding the BTN2-BTN3 heteromeric complex of the disclosure, the multivalent BTN2-BTN3 complex of the disclosure, the modified BTN3, or the modified BTN2 of the disclosure.

In one embodiment, the one or more nucleic acids comprise:
i) a nucleic acid sequence having at least 70% identity to SEQ ID NO: 5, 31, 35, 39, or 47; and/or
ii) ii) a nucleic acid sequence having at least 70% identity to SEQ ID NO: 6, 32, 36, 40, or 48.

The present disclosure also provides one or more vectors comprising one or more nucleic acids encoding the BTN2-BTN3 heteromeric complex of the disclosure, or the multivalent BTN2-BTN3 complex of the disclosure, the modified BTN3 of the disclosure, or the modified BTN2 of the disclosure.

The present disclosure also provides a host cell comprising the BTN2-BTN3 heteromeric complex of the disclosure, the multivalent BTN2-BTN3 complex of the disclosure, the modified BTN3 of the disclosure, the modified BTN2 of the disclosure, the one or more nucleic acids of the disclosure, or the one or more vectors of the disclosure.

The present disclosure also provides for a composition comprising one or more of:
(i) the BTN2-BTN3 heteromeric complex of the disclosure;
(ii) the multivalent BTN2-BTN3 complex of the disclosure;
(iii) the modified BTN3 of the disclosure;
(iv) the modified BTN2 of the disclosure;
(v) the one or more nucleic acids of the disclosure;
(vi) the one or more vectors of the disclosure; and
(vii) the cell of the disclosure; and
(viii) optionally, one or more pharmaceutically acceptable excipients.

The present disclosure also provides a method for modifying a cell, the method comprising:
(i) providing the cell; and
(ii) introducing the one or more nucleic acids of the disclosure; or the one or more vectors of the disclosure into the cell; and
(iii) optionally, culturing the cell.

The present disclosure also provides a cell obtained by said method.

The present disclosure also provides a method for activating γδ T cells that express a δ2⁺ TCR, the method comprising contacting the cells with the BTN2-BTN3 heteromeric complex of the disclosure, the multivalent BTN2-BTN3 complex of the disclosure, or the modified BTN3 of the disclosure; and, optionally, administering the activated γδ T cells to a subject in need thereof. In some embodiments, the BTN3 or BTN2-BTN3 complex of the disclosure is immobilised (e.g., on a tissue culture plate by, for example, electrostatic forces) and a sample comprising γδ T cells (e.g., a peripheral blood sample such as a PBMC sample) is contacted with the plate bound BTN3 or BTN2-BTN3 complex for a sufficient time such as 48 hours to activate the γδ T cells. The activated γδ T cells may be subsequently isolated and optionally administered to a subject in need thereof.

The present disclosure also provides for use of the modified TCR or binding fragment thereof of the disclosure, for example a soluble TCR, the one or more nucleic acids of the disclosure, the one or more vectors of the disclosure, the cell of disclosure, or the composition of the disclosure as a medicament.

The present disclosure also provides for use of the BTN2-BTN3 heteromeric complex of the disclosure, the multivalent BTN2-BTN3 complex of the disclosure, the modified BTN3 of the disclosure, the one or more nucleic acids of the disclosure, the one or more vectors of the disclosure, the cell of disclosure, or the composition of the disclosure as a medicament for use in detection, diagnosis, prognosis, prevention and/or treatment of cancer or an infection.

The present disclosure also provides a method of preventing, treating, delaying the progression of, preventing a relapse of, or alleviating a symptom of a cancer or an infection, wherein the method comprises administering the BTN2-BTN3 heteromeric complex of the disclosure, the multivalent BTN2-BTN3 complex of the disclosure, the modified BTN3 of the disclosure, the one or more nucleic acids of the disclosure, the one or more vectors of the disclosure, the cell of disclosure, or the composition of the disclosure to a subject in need thereof.

The present disclosure also provides a method of detecting T cells in a subject, comprising:
(i) providing a sample from the subject; and
(ii) contacting the sample with the BTN2-BTN3 heteromeric complex of the disclosure, the multivalent BTN2-BTN3 complex of the disclosure, or the modified BTN3 of the disclosure to form a complex with a TCR (for example a Vδ2⁺ TCR); and
(iii) detecting the complex.

The present disclosure also provides a method of detecting the presence of a cancer or an infection in a subject in vitro, comprising:
(i) providing a sample from the subject; and
(ii) contacting the sample with the BTN2-BTN3 heteromeric complex of the disclosure, the multivalent BTN2-BTN3 complex of the disclosure, or the modified BTN3 of the disclosure to form a complex; and
(iii) detecting the complex, wherein detection of the complex is indicative of the presence of the cancer or infection in the subject.

In some embodiments, the sample is a peripheral blood sample. The sample may be further processed, for example, the γδ T cells may be purified from the peripheral blood sample using any purification method known in the art, for example, positive or negative enrichment using magnetic beads, and/or cell sorting. The sample may be contacted with BTN3 or a BTN2-BTN3 complex of the disclosure, preferably labelled with a detectable tag (e.g., Phycoerythrin (PE) for flow cytometry) and the complex of the BTN3 or BTN2-BTN3 with Vδ2+ TCRs detected, by for example, flow cytometry.

In other embodiments, the sample is a lymph node or tissue sample (e.g., tumour tissue sample). In some embodiments, BTN3 or a BTN2-BTN3 complex of the disclosure, preferably labelled with a detectable tag (e.g., an AlexaFluor dye for microscopy imaging such as AF647) can be used to detect and/or quantify infiltrate of Vδ2+ cells (e.g., γδ T cells) into the tumour microenvironment.

The present disclosure also provides for use of the one or more nucleic acids of the disclosure, or the one or more vectors of the disclosure for generating modified cells, for example, antigen presenting cells.

The present disclosure also provides for use of the BTN2-BTN3 heteromeric complex of the disclosure, the multivalent BTN2-BTN3 complex of the disclosure, the modified BTN3 of the disclosure, the one or more nucleic acids of the disclosure, the one or more vectors of the disclosure, the cell of disclosure, or the composition of the disclosure for use in prevention and/or treatment of an autoimmune disease, transplantation rejection, graft versus host disease, or graft versus tumour effect.

The present disclosure also provides a method of preventing, treating, delaying the progression of, preventing a relapse of, or alleviating a symptom of an autoimmune disease, transplantation rejection, graft versus host disease, or graft versus tumour effect, wherein the method comprises administering the BTN2-BTN3 heteromeric complex of the disclosure, the multivalent BTN2-BTN3 complex of the disclosure, the modified BTN3 of the disclosure, the one or more nucleic acids of the disclosure, the one or more vectors of the disclosure, the cell of disclosure, or the composition of the disclosure to a subject in need thereof.

### BRIEF DESCRIPTION OF DRAWINGS

FIG. 1. (A) Size exclusion (S200 16/600) gel filtration chromatography of BTN2A1 (black) and BTN3A1 (grey) ectodomains produced in MGAT1-deficient Expi293F cells. Larger elution volume indicates smaller protein size. (B) Overlay of BTN2A1 V-dimer from apo structure and BTN3A1 V-dimer structures (PDB code 4F80). (C) Surface representation of BTN2A1 depicting the head-to-tail dimer interface in light grey and the V-dimer interface in dark grey. Glycans depicted as sticks. (D) Overlay of BTN2A1 head-to-tail dimers derived from the apo BTN2A1 and γδTCR-BTN2A1 (unliganded copy of BTN2A1) crystal structures. (E) Overlay of BTN2A1 V-dimer structures derived from the apo and γδTCR-liganded crystal structures. (F) Cartoon of BTN2A1 depicting the CFG face of the IgV domain and the ABED face of the IgV domain (G) Surface representation of BTN2A1 and G115 Vγ9Vδ2 TCR depicting the interfaces from the γδTCR-BTN2A1 complex. (H) Cartoon overlay of apo and liganded BTN2A1, depicting the conformational changes to residues that are involved in binding to Vγ9Vδ2⁺ TCR (shown as sticks).

FIG. 2. BTN2A1 engages the side of Vγ9. (A) Surface and cartoon representation of the apo-BTN2A1 crystal structure. (B) The BTN2A1 V-dimer (left) and cis (middle) or trans (right) interpretation of the head-to-tail homodimer. (C) Surface and cartoon representation of the BTN2A1-Vγ9Vδ2⁺ TCR clone G115 crystal structure. G115 TCRδ; G115 TCRy; liganded BTN2A1; unliganded BTN2A1. (D) Comparison with a representative pHLA Class I-αβTCR (left, PDB code 1QSE (35)) and CD1d-α-GalCer-clone 9C2 Vγ5Vδ1⁺ TCR (right, PDB code 4LHU (36)). Molecular contacts between the Vγ9Vδ2⁺ clone G115 TCR and BTN2A1 ectodomains showing the (E) Arg20γ, (F) Glu70γ and His85γ, (G) Lys13γ and Lys17γ, (H) Ser16γ and Thr18γ side and/or main chains and their BTN2A1 contacts as sticks. H-bonds and salt-bridges, grey; VDW and cation-π, black; 2mFo-DFc electron density map contoured at 1σ.

FIG. 3. (A) BTN2A1 tetramer, BTN3A1 tetramer, control mouse CD1d tetramer, or SAv-PE staining of human HEK293T cells transfected with plasmids co-encoding GFP and either G115 Vγ9Vδ2⁺ or control 9C2 Vγ5Vδ1⁺γδTCRs. Plots gated on GFP⁺ cells. Data from one of 10 independent experiments. Inset—median fluorescence intensity (MFI) of PE parameter. (B) γδTCR tetramer or SAv control staining of HEK293T BTN2A.BTN3A^{KO} cells transfected with plasmids co-encoding GFP and either BTN2A1, BTN3A1 or control BTNL3, which were pre-incubated with anti-BTN3A mAb 20.1 or isotype control (mouse IgG1,κ) antibody. Plots gated on GFP⁺ cells. Inset—MFI of PE parameter of mAb 20.1-treated cells within the representative GFP⁺ gate. Representative of one of two independent experiments. MFI of chimeric γδTCR tetramer staining of (C) gated GFP⁺ BTN2A1-transfected or (D) GFP⁺ BTN3A1-transfected NIH-3T3 cells. Inset plots depict parent cell gating. Graphs are presented as mean±SEM. N≥3, where each point represents an independent experiment. MFI of G115 γδTCR tetramer staining of (E) GFP⁺ BTN2A1-transfected or (F) GFP⁺ BTN3A1-transfected NIH-3T3 cells. Graphs depict mean±SEM. N≥3, where each point represents an independent experiment. (G) GFP⁺ BTN2A1-transfected or GFP⁺ BTN3A1-transfected NIH-3T3 cells were stained with streptavidin (SAv)-PE control, Vγ9Vδ2⁺‘G115 WT’, ‘G115 Lys53δ-Ala’, ‘TCR 6 WT’ or ‘TCR 6 Lys53δ-Ala’ TCR tetramers. Representative one of two independent experiments. (H) GFP⁺ BTN2A1-transfected or GFP⁺ BTN3A1-transfected NIH-3T3 cells were stained with isotype control (MOPC21)-AF647 or anti-BTN3A (20.1)-AF647 antibodies followed by control SAv-PE, Vγ9Vδ2 ‘G115 WT’ or ‘G115 Lys53δ-Ala’ TCR tetramer-PE staining. Cells were examined for FRET in the YG 670/30 channel by flow cytometry.

FIG. 4. BTN3A1 supports binding to the apical surface of the Vγ9Vδ2⁺γδTCR. (A) Vγ9Vδ2⁺ TCR tetramer-PE (clones TCR3, TCR6, TCR7 and G115) or streptavidin (SAv.) control staining of mouse NIH-3T3 cells transfected with BTN2A1, BTN3A1 or no DNA following pre-incubation with anti-BTN3A mAb clones 20.1 (grey) or 103.2 (dark grey), or isotype control (IgG1,κ, black). (B) Staining of BTN2A1, BTN3A1 or control BTNL3-transfected NIH-3T3 cells with chimeric γδTCR tetramers comprised of the TCR6, TCR7 or G115 pAg-reactive γ-chains, plus either the pAg-reactive Vδ2⁺ or the 9C2 Vδ1⁺ δ-chains±anti-BTN3A mAb 20.1 (grey) or isotype control (IgG1,κ, black). Median fluorescence intensity (MFI) of PE for mAb 20.1-treated cells (grey numbers) or isotype control (IgG1,κ)-treated BTN3A1⁺ cells (black numbers) shown within the depicted GFP⁺ gate. (C) Wild-type or mutant G115 Vγ9Vδ2⁺ TCR tetramer staining, or control mouse CD1d-α-GalCer (mCD1d tet.) or streptavidin alone (SAv) staining of NIH-3T3 cells transfected as in (B)±anti-BTN3A mAb clone 20.1 (grey) or isotype control (IgG1,κ, black). Triple-γ mutant comprises Arg20γ-Ala/Glu70γ-Ala/His85γ-Ala mutations. Cartoon inset depicts the locations of BTN2A1-epitope (dark grey star) and the ligand-two epitope (light grey star). Representative of one of three independent experiments. MFI of PE for mAb 20.1-treated cells (red numbers) or isotype control (IgG1,κ)-treated BTN3A1⁺ cells (black numbers) shown within the depicted GFP⁺ gate.

FIG. 5. (A) and (B) CD69-PE expression on G115 WT or mutant Vγ9Vδ2⁺, 9C2 Vγ5Vδ1⁺γδTCR or parental (TCR⁻) J.RT3-T3.5 Jurkat cells after overnight co-culture with LM-MEL-75 APCs in the presence or absence of 40 μM zoledronate. Graphs are presented as mean±SEM. N=2, where each point represents an independent experiment. (C) Surface representation of G115 Vγ9Vδ2⁺ TCR depicting the interacting and gatekeeper or uninvolved residues based on Jurkat activation assays.

FIG. 6. (A) Representation of BTN2A1-BTN3A1-zipper complex. (B-C) (LHS) BTN2A1-BTN3A1 complex was expressed in Expi293F cells and purified by (B) affinity (NiNTA) and (C) size exclusion (S200) chromatography. (RHS) Protein purified in boxes run over SDS-PAGE to confirm identity. MM—molecular weight marker; 2A1-BTN2A1-acid zipper (AZ)-His6; 3A1-BTN3A1-basic zipper (BZ)— Biotin ligase tag. (D) Reactivity of anti-BTN2A1 (clone 259), anti-BTN3A (clone 103.2), mouse IgG2a isotype control (clone BM4-2a), or mouse IgG1 isotype control (clone MOPC-21) to immobilized BTN1A1, BTN2A1, BTN3A1 or BTN2A1-BTN3A1-zipper ectodomains by ELISA. (E) BTN2A1-BTN3A1-zipper complex was crystallized, resolubilized and run on SDS-PAGE, along with crystal wash buffer and input BTN2A1-BTN3A1-zipper complex. MM—molecular weight marker; 2A1-BTN2A1-AZ-His6; 3A1-BTN3A1-BZ-Biotin ligase tag. (F) BTN2A1-, BTN3A1-, BTN2A1-BTN3A1 complex- or control mouse CD1d-ectodomain tetramers, or control streptavidin-PE (SAv) staining of HEK293T cells co-transfected with CD3 plus G115 Vγ9Vδ2+ TCR wild-type, His85γ-Ala, Glu52δ-Ala, Lys53δ-Ala or control 9C2 Vγ5Vδ1+ TCR. N≥5 independent experiments. *P<0.05, **P<0.01; BTN2A1-BTN3A1 complex tetramer binding to Vγ9Vδ2+ TCRs tested by Kruskal-Wallis test with two-stage step-up multiple correction method of Benjamini, Krieger and Yekutieli.

FIG. 7. BTN3A1 is a ligand for the γδTCR. (A) BTN2A1-, BTN3A1-, BTN2A1-BTN3A1 complex- or control mouse CD1d-ectodomain tetramers, or streptavidin alone (SAv) versus anti-CD3 staining of HEK293T cells co-transfected with CD3 plus G115 Vγ9Vδ2⁺ TCR wild-type, His85γ-Ala, Glu52δ-Ala, Lys53δ-Ala or control 9C2 Vγ5Vδ1⁺ TCR. Cartoon inset depicts the relative locations of BTN2A1-epitope mutants or ligand-two epitope mutants. Representative of one of three independent experiments. Inset—median fluorescence of PE parameter. (B) Sensorgrams (left) and saturation plots (right) depicting binding of soluble G115 Vγ9Vδ2⁺ TCR wild-type (302-4.2 μM), His85γ-Ala (410-1.6 μM), Glu52δ-Ala (370-5.8 μM) and Lys53δ-Ala (295-4.6 μM) to immobilised BTN2A1 ectodomain homodimer, BTN3A1 ectodomain homodimer and BTN2A1-BTN3A1 ectodomain complex, as measured by surface plasmon resonance. K_{D}, dissociation constant calculated at equilibrium ±SEM, derived from the mean of n=2 (WT and His85γ) or n=3 (Glu52δ and Lys53δ) independent experiments.

FIG. 8. BTN2A1 and BTN3A1 directly associate and form heteromers. (A) Sensorgrams (left) and saturation plots (right) depicting binding of soluble monomeric BTN2A1 ectodomain (top row, 890-28 μM), homodimeric BTN3A1 ectodomain (middle row, 1,520-24 μM), or monomeric BTN3A1 IgV domain (bottom row, 1,590-25 μM) to immobilised BTN2A1 ectodomain homodimer (red) or BTN3A1 ectodomain homodimer (blue), as measured by surface plasmon resonance. Insert graphs depict Scatchard plots. K_{D}, dissociation constant calculated at equilibrium ±SEM, derived from the mean of n=2 independent experiments each shown separately as dotted and close lines on the saturation plots. (B) The BTN2A1-BTN3A1 ectodomain complex crystal structure, showing the asymmetric unit as a surface and the V-dimers as a cartoon. (C) Surface and cartoon representation of the BTN2A1 V-dimer-BTN3A1 V-dimer repeating unit within the crystal structure. Molecular contacts between BTN2A1 and BTN3A1 ecdodomains showing the (D) BTN2A1 Arg56 and Glu35, (E) Phe43 and Glu107, (F) Phe43 N atom and Ser44, and (G) Glu35, Lys51 and Gln100 side and/or main chains and their BTN3A1 contacts as sticks. H-bonds and salt-bridges, grey; cation-rr, black. (H) Association between BTN2A1 and BTN3A1 ectodomains on the cell surface of mouse NIH-3T3 fibroblasts co-expressing wild-type BTN2A1 and individual BTN3A1 mutants, as determined by mean percentage ±SEM of FRET+ cells between anti-BTN2A1-AF647 (clone 259) and anti-BTN3A-PE (clone 103.2). Controls (right) depict FRET between CD80 and PD-L1, or BTN2A1 and PD-L1. n=6 where each point represents an individual experiment, except for controls where n=3. NA, not applicable since BTN3A surface expression was too low to measure FRET.

FIG. 9. (A) Comparison of the apo BTN3A1 homodimer (PDB code 4F80) with BTN3A1 homodimer from the BTN2A1-BTN3A1-zipper complex, and a comparison of apo BTN2A1 homodimer with BTN2A1 homodimer from the BTN2A1-BTN3A1-zipper complex. (B) Surface representation of BTN2A1 and BTN3A1 depicting the regions that are contacting each other.

FIG. 10. Summary of the effect of single-residue mutations within the (A) IgV domain or (B) IgC domain of BTN3A1 on anti-BTN3A reactivity (mAb clones 103.2 and 20.1) as well as binding in cis to BTN2A1 as measured by FRET, and binding to G115 γδTCR tetramer. (C) Förster resonance energy transfer (FRET) between anti-BTN2A1 (clone 259) and anti-BTN3A (clone 103.2) mAb staining on gated BTN2A1⁺ BTN3A1⁺ NIH-3T3 cells, 48 h after co-transfection with WT BTN2A1 plus the indicated BTN3A1 mutant, or as irrelevant controls, BTN2A1 plus PD-L2 or BTN3A1 plus CD80. Mutants in dark grey were excluded from analysis due to diminished BTN3A1 staining. Mutants in light grey are those which reduced FRET levels. Representative one of six independent experiments. (D) Surface representation of BTN3A1 V-dimer depicting residue side chains that upon mutation led to an abrogation of BTN3A1 association with BTN2A1 (grey), or those which did not impact the interaction with BTN2A1 (black), as determined by the FRET assay (left). The BTN3A1 surface on the right depicts atoms that contacted BTN2A1 based on the crystal structure (reproduced from FIG. S5B).

FIG. 11. (A) G115 tetramer-PE staining of BTN3A1 WT or mutant-transfected NIH-3T3 cells following pre-incubation with anti-BTN3A-AF647 (mAb clone 20.1). Mutants in grey were excluded from analysis due to diminished BTN3A1 mAb 20.1 staining. Mutants in light grey are those which impaired G115 tetramer staining. Representative of one of three independent experiments. (B) CD25-PE expression on purified pre-expanded Vδ2⁺γδ T cells following co-culture with NIH-3T3 cells that were co-transfected with BTN2A1 plus the indicated BTN3A1 mutant, or alternatively control BTNL3 plus BTNL8, ±zoledronate (5 μM) for 24 h. Data are from one of three independent experiments, each with two donors. (C) Surface of BTN2A1 V-dimer depicting residues that contact BTN3A1 based on the BTN2A1-BTN3A1 crystal structure, residues that contact Vγ9Vδ2⁺ TCR based on the G115 TCR-BTN2A1 crystal structure, and residues that overlap and contact both.

FIG. 12. BTN3A1 IgV domain interacts with Vγ9Vδ2⁺ TCR. (A) G115 Vγ9Vδ2⁺ TCR tetramer-PE staining of mouse NIH-3T3 fibroblasts transfected with either wild-type BTN3A1 or the indicated mutants, following pre-treatment with anti-BTN3A1-AF647 (clone 20.1) antibody. SAv, streptavidin-PE control staining of wild-type BTN3A1⁺ cells. Bar graphs depict median fluorescence intensity (MFI)±SEM. Dotted lines represents 90-98% reduction and >98% reduction in MFI. Inset: surface representation of BTN3A1 V-dimer with mutations to residues that led to an abrogation of the anti-BTN3A antibody (20.1)-dependent G115 tetramer interaction coloured in dark grey (>98% reduction), light grey (90-98% reduction), or grey (<90% reduction). n=3, where each point represents an independent experiment. NA, not applicable since BTN3A surface expression was too low to measure G115 tetramer staining. (B) Change in CD25 expression (normalized to unstimulated control for each sample) on purified in vitro-expanded Vδ2⁺γδ T cells co-cultured for 24 h with 5 μM zoledronate and mouse NIH-3T3 fibroblast APCs transfected with wild-type BTN2A1 and individual BTN3A1 mutants. Bar graphs depict mean±SEM. Dotted line represents >50% reduction in activation compared to BTN2A1-BTN3A1 WT. NA=data not available since BTN3A1 levels were too low to induce zoledronate-dependent activation of γδ T cells. Data are from 2-3 independent experiments each with n=1-2 different donors. **P<0.01, ***P<0.001, ****P<0.0001, by two-way ANOVA with Šidák multiple comparison correction. Inset: surface representation of BTN3A1 V-dimer with mutations to residues that led to an abrogation of zoledronate-dependent Vδ2⁺γδ T cell activation shown in grey, or did not impact Vδ2⁺γδ T cell activation shown in dark grey.

FIG. 13. BTN2A1 and BTN3A1 must disengage in order to bind Vγ9Vδ2⁺ TCR. (A) BTN2A1 tetramer, BTN2A1-BTN3A1 WT complex tetramer, BTN2A1-BTN3A1 Glu135-Ala complex tetramer, or control streptavidin alone (SAv), versus anti-CD3 staining on HEK293T cells co-transfected with CD3 plus G115 Vγ9Vδ2⁺ TCR wild-type, Glu52δ-Ala, or control 9C2 Vγ5Vδ1⁺ TCR. Inset—median fluorescence intensity (MFI) of PE parameter. Representative of one of six independent experiments. (B) Sensorgrams (top) and saturation plots (bottom) depicting binding of soluble G115 Vγ9Vδ2⁺ TCR wild-type (181-2.8 μM), Glu52δ-Ala 243-3.8 μM) and Lys53δ-Ala (139-2.2 μM) to immobilised BTN2A1-BTN3A1 wild-type (left), BTN2A1-BTN3A1 Glu135-Ala (middle) or BTN2A1 Gly102-Cys-BTN3A1 Asp103-Cys (right) complexes. K_{D}, dissociation constant calculated at equilibrium ±SEM, derived from the mean of two independent experiments. (C) G115 Vγ9Vδ2⁺ TCR tetramer-PE, or control streptavidin-PE (SAv) staining of mouse NIH-3T3 fibroblasts co-transfected with BTN2A1 and BTN3A1 wild-type or cysteine mutants in the depicted combinations. Representative of one of two independent experiments. (D) G115 Vγ9Vδ2⁺ TCR tetramer-PE, or control streptavidin-PE (SAv) staining of mouse NIH-3T3 fibroblasts co-transfected with the indicated BTN2A1 and BTN3A1 cysteine mutant pairs, or control BTNL3 plus BTNL8, following pre-treatment of cells with graded concentrations of dithiothreitol (DTT). Inset—MFI of PE parameter. Representative of one of four independent experiments.

FIG. 14. (A) Structure of BTN2A1-BTN3A1 depicting the locations of the two cysteine mutant pairs. (B) G115 tetramer-PE staining of NIH-3T3 fibroblasts co-transfected with either WT or Cys-mutant BTN2A1 plus BTN3A1, or control BTNL3 plus BTNL8, following pre-incubation of the cells with DTT at indicated concentrations. Graphs are presented as mean±SEM. Data pooled from 3-4 separate experiments. (C) Predicted structure of the BTN2A1-BTN3A1 complex containing a disulfide bond between BTN2A1 and BTN3A1 molecules, based on the BTN2A1-BTN3A1 ectodomain complex crystal structure. (D) 2D class averages of negatively stained soluble BTN2A1 Gly102-Cys-BTN3A1 Asp103-Cys ectodomain complex. (E) BTN2A1 tetramer, BTN2A1-BTN3A1 complex tetramer, BTN2A1 Gly102-Cys-BTN3A1 Asp103-Cys complex tetramer, or control tetramer (mouse CD1d) or SAv.-PE alone staining of HEK293T cells transfected with either G115 Vγ9Vδ2⁺ or control 9C2 Vγ5V1⁺γδTCRs. Where indicated, BTN molecules were pre-treated with 5 mM DTT prior to being tetramerised with SAv-PE. Representative one of one or four independent experiments. (F) SDS-PAGE of BTN monomers treated with DTT as in (E).

FIG. 15. Proposed model of Vγ9Vδ2⁺ TCR interacting with the cryptic BTN2A1-BTN3A1 complex on APCs following anti-BTN3A mAb 20.1 antibody treatment. Created with BioRender.com.

FIG. 16. (A) Surface BTN2A1 expression (clone 259) on HEK293T BTN2A^{KO}.BTN3A^{KO} cells that were transfected with BTN2A1 WT or the indicated BTN2A1 intracellular domain mutants, or control BTNL3. Representative from one of two experiments. (B) Representative plots and (C) mean±SEM of CD25-PE expression on purified pre-expanded Vδ2⁺γδ T cells following co-culture with HEK293T BTN2A^{KO}.BTN3A^{KO} cells that were co-transfected with BTN3A1 plus the indicated BTN2A1 mutant, or alternatively, control BTNL3 alone or BTN3A1 alone, zoledronate (5 μM) for 24 h. Replicates with low transfection efficiency (<10% GFP⁺) were excluded from analysis. Data are from three independent experiments each with n=2 different donors. **, p<0.01 by two-way ANOVA with Šidák multiple comparison correction. Insert, molecular model of the BTN2A1 B30.2 intracellular domain generated by AlphaFold v2 with functionally important residues shown in red.

FIG. 17. Introduction of Glu106-Ala mutation into BTN3A1 induces BTN2A1-BTN3A1 complex reactivity to Vδ2⁺ T cells. (A) BTN2A1-BTN3A1-zipper complex tetramer-PE, or (B) BTN2A1-BTN3A1 Glu106-Ala-zipper complex tetramer-PE, or (C) BTN2A1 Gly102-Cys-BTN3A1 Asp103-Cys-zipper complex tetramer-PE staining of purified primary Vδ2⁺ cells derived from a healthy blood donor, versus anti-CD3-APC, following nucleofection under the indicated conditions. RNP, ribonucleoprotein; guide 1 and guide 2, sgRNA targeting Vδ2 CDR2δ region; template, single-stranded DNA repair template encoding a Lys53δ-Ala mutation.

FIG. 18. Platebound BTN2A1 plus BTN3A1 ectodomains activates Vδ2⁺γδ T cells in vitro. CD25 expression on expanded pre-enriched Vδ2+γδ T cells following overnight culture on plates coated with the indicated proteins.

FIG. 19. Modified C-terminal linkers and zipper domains facilitate BTN2A1 association with BTN3A1 and reactivity to γδTCR. NIH-3T3 cells were transfected with either Vγ9Vδ2+ TCR clone G115, or control 9C2 TCR, and stained with PE-conjugated tetramers of the depicted BTN molecules, or control streptavidin (SAv) or mouse CD1 d-alpha-GalCer (mCD1 d). (A) BTN2A1-BTN3A1 ectodomain complex tetramers tethered with C-terminal high affinity leucine (acidic/basic) zippers fused to the BTN ectodomains with a ‘long’ (13mer) or a ‘short’ (3mer) glycine/serine-rich linker. (B) BTN2A1-BTN3A1 ectodomain complex tetramers tethered with C-terminal high affinity leucine (acidic/basic) zippers with a ‘long’ glycine/serine-rich linker (as per A), or with C-terminal reduced affinity leucine (acidic/basic) zippers with a ‘long’ glycine/serine-rich linker.

FIG. 20. Schematic of different BTN2A1-BTN3A1 constructs that were produced.

FIG. 21. Introduction of Ser44-Arg mutation in BTN2A1 induces BTN2A1-BTN3A1 complex interaction with Vγ9Vδ2⁺ TCR. (A) G115 Vγ9Vδ2 TCR tetramer (left), G115 His85γ-Ala Vγ9Vδ2 TCR tetramer (middle), streptavidin-phycoerythrin (PE) control (right) staining versus GFP, on mouse NIH-3T3 cells transfected with the indicated full-length BTN genes. Black gate depicts TCR tetramer staining that is dependent on BTN2A1-BTN3A1 co-expression. (B) G115 His85γ-Ala Vγ9Vδ2 TCR tetramer versus GFP on NIH-3T3 cells co-transfected with full-length BTN2A1 and BTN3A1 genes, +/− zoledronate (100 μM). Numbers on each plot represent mean fluorescence intensity of TCR tetramer on cells within the black gates. Data are from one of two representative experiments.

### KEY TO SEQUENCE LISTING

SEQ ID NO: 1 is an amino acid sequence of variable region of BTN2A1 isoform 1.
SEQ ID NO: 2 is an amino acid sequence of variable region of BTN3A1 isoform 1.
SEQ ID NO: 3 is an amino acid sequence of mutated (Glu106-Ala) variable region of BTN3A1 isoform 1.
SEQ ID NO: 4 is an amino acid sequence of mutated (Glu106-Ala) of BTN3A1 isoform 1.
SEQ ID NO: 5 is a nucleic acid sequence of variable region of BTN2A1 isoform 1.
SEQ ID NO: 6 is a nucleic acid sequence of variable region of BTN3A1 isoform 1.
SEQ ID NO: 7 is a nucleic acid sequence of mutated (Glu106-Ala) variable region of variable region of BTN3A1 isoform 1.
SEQ ID NO: 8 is a nucleic acid sequence of mutated (Glu106-Ala) BTN3A1 isoform 1.
SEQ ID NO: 9 is an amino acid sequence of human BTN2A1 isoform 1.
SEQ ID NO: 10 is an amino acid sequence of human BTN2A1 isoform 2.
SEQ ID NO: 11 is an amino acid sequence of human BTN2A1 isoform 3.
SEQ ID NO: 12 is an amino acid sequence of human BTN2A1 isoform 4.
SEQ ID NO: 13 is an amino acid sequence of human BTN2A1 isoform 5.
SEQ ID NO: 14 is an amino acid sequence of human BTN2A1 isoform 6.
SEQ ID NO: 15 is an amino acid sequence of human BTN3A1 isoform 1.
SEQ ID NO: 16 is an amino acid sequence of human BTN3A1 isoform 2.
SEQ ID NO: 17 is an amino acid sequence of human BTN3A1 isoform 3.
SEQ ID NO: 18 is an amino acid sequence of human BTN3A1 isoform 4.
SEQ ID NO: 19 is a nucleic acid sequence of human BTN2A1 isoform 1.
SEQ ID NO: 20 is a nucleic acid sequence of human BTN2A1 isoform 2.
SEQ ID NO: 21 is a nucleic acid sequence of human BTN2A1 isoform 3.
SEQ ID NO: 22 is a nucleic acid sequence of human BTN2A1 isoform 4.
SEQ ID NO: 23 is a nucleic acid sequence of human BTN2A1 isoform 5.
SEQ ID NO: 24 is a nucleic acid sequence of human BTN2A1 isoform 6.
SEQ ID NO: 25 is a nucleic acid sequence of human BTN3A1 isoform 1.
SEQ ID NO: 26 is a nucleic acid sequence of human BTN3A1 isoform 2.
SEQ ID NO: 27 is a nucleic acid sequence of human BTN3A1 isoform 3.
SEQ ID NO: 28 is a nucleic acid sequence of human BTN3A1 isoform 4.
SEQ ID NO: 29 is amino acid sequence of BTN2A1 of construct #1 (long linker version with high affinity zippers).
SEQ ID NO: 30 is an amino acid sequence of BTN3A1 of construct #1 (long linker version with high affinity zippers).
SEQ ID NO: 31 is a nucleic acid sequence of BTN2A1 of construct #1 (long linker version with high affinity zippers).
SEQ ID NO: 32 is a nucleic acid sequence of BTN3A1 of construct #1 (long linker version with high affinity zippers).
SEQ ID NO: 33 is amino acid sequence of BTN2A1 of construct #2 (short linker version).
SEQ ID NO: 34 is an amino acid sequence of BTN3A1 of construct #2 (short linker version).
SEQ ID NO: 35 is a nucleic acid sequence of BTN2A1 of construct #2 (short linker version).
SEQ ID NO: 36 is a nucleic acid sequence of BTN3A1 of construct #2 (short linker version).
SEQ ID NO: 37 is amino acid sequence of BTN2A1 of construct #3 (long linker version with reduced affinity zippers).
SEQ ID NO: 38 is an amino acid sequence of BTN3A1 of construct #3 (long linker version with reduced affinity zippers).
SEQ ID NO: 39 is a nucleic acid sequence of BTN2A1 of construct #3 (long linker version with reduced affinity zippers).
SEQ ID NO: 40 is a nucleic acid sequence of BTN3A1 of construct #3 (long linker version with reduced affinity zippers).
SEQ ID NO: 41 is amino acid sequence of BTN2A1 of construct #4 (BTN2A1 Gly102-Cys-BTN3A1 Asp103-Cys-zipper).
SEQ ID NO: 42 is an amino acid sequence of BTN3A1 of construct #4 (BTN2A1 Gly102-Cys-BTN3A1 Asp103-Cys-zipper).
SEQ ID NO: 43 is a nucleic acid sequence of BTN2A1 of construct #4 (BTN2A1 Gly102-Cys-BTN3A1 Asp103-Cys-zipper).
SEQ ID NO: 44 is a nucleic acid sequence of BTN3A1 of construct #4 (BTN2A1 Gly102-Cys-BTN3A1 Asp103-Cys-zipper).
SEQ ID NO: 45 is amino acid sequence of BTN2A1 of construct #5 (BTN2A1-BTN3A1 Glu106-Ala-zipper).
SEQ ID NO: 46 is an amino acid sequence of BTN3A1 of construct #5 (BTN2A1-BTN3A1 Glu106-Ala-zipper).
SEQ ID NO: 47 is a nucleic acid sequence of BTN2A1 of construct #5 (BTN2A1-BTN3A1 Glu106-Ala-zipper with long linker).
SEQ ID NO: 48 is a nucleic acid sequence of BTN3A1 of construct #5 (BTN2A1-BTN3A1 Glu106-Ala-zipper with long linker).
SEQ ID NO: 49: acidic zipper
SEQ ID NO: 50: basic zipper
SEQ ID NO: 51: acidic zipper reduced affinity
SEQ ID NO: 52: basic zipper reduced affinity
SEQ ID NO: 53: short linker
SEQ ID NO: 54: long linker with thrombin cleavage site
SEQ ID NO:55-56: Guide RNAs
SEQ ID NO:57-92: Primers
SEQ ID NO:93-94: Guide RNAs
SEQ ID NO:95: Alt-R HDR oligonucleotide sequence
SEQ ID NO: 96 is an amino acid sequence of mutated (Ser44-Arg) variable region of BTN2A1 isoform 1.
SEQ ID NO: 97 is an amino acid sequence of mutated (Ser44-Arg) of BTN2A1 isoform 1.
SEQ ID NO: 98 is a nucleic acid sequence of mutated (Ser44-Arg) variable region of BTN2A1 isoform 1.
SEQ ID NO: 99 is a nucleic acid sequence of mutated (Ser44-Arg) BTN2A1 isoform 1.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

The term “T cell receptor” or “TCR” as used herein refers to a receptor capable of specifically interacting with a target antigen and includes full length TCRs and antigen binding fragments or portions thereof, native TCRs as well as TCR variants, fragments and constructs. TCRs of the disclosure can be isolated or may be made synthetically or recombinantly. The term includes heterodimers comprising, for example, TCR δ and γ chains, as well as multimers and single chain constructs; optionally comprising further domains and/or moieties.

A TCR is generally considered to comprise two chains, for example, a γ chain and a δ chain. Each chain comprises a variable region (e.g., Vγ and Vδ) and optionally, one or more of diversity (D), joining (J) and constant regions (e.g., Cγ and/or Cδ).

The variable regions can be further subdivided into regions of hypervariability, termed complementarity determining regions (CDRs), interspersed with regions that are more conserved, termed framework regions (FR). For example, the variable region comprises three CDRS and three or four FRs (e.g., FR1, FR2, FR3 and optionally FR4). Each variable region comprises a binding domain that interacts with an antigen. One or more of CDRs on each chain may be involved in antigen binding. The CDR3s are highly diverse due to V(D)J combinatorial diversity as well as non-template nucleotide modifications, and often form part of the primary antigen binding region. The CDR3γ is semi-invariant in length and composition, and a lysine within the CDR3γ at position 108, encoded by TRGJP, is important for γδ T cell-mediated responses to phosphoantigens.

As used herein, the term “TCR” further refers to a TCR that is expressed on the surface of a cell including a T cell or a cell other than a T cell or an isolated or soluble TCR.

Where not expressly stated, and unless the context indicates otherwise, the term “TCR” also includes an antigen-binding fragment or an antigen-binding portion of any TCR disclosed herein and includes a monovalent and a divalent fragment or portion, a soluble TCR and a single chain TCR. The term “TCR” is not limited to naturally occurring TCRs bound to the surface of a T cell.

An “antigen binding fragment” or “antigen binding portion” refers to any portion of a TCR less than the whole that retains antigen binding. An “antigen binding fragment” or “antigen binding portion” can include the antigenic complementarity determining regions (CDRs).

An “antigen” refers to any molecule, for example a (poly-) peptide that is capable of being bound by a TCR or binding fragment thereof. In the context of the present invention the term “binding domain” in particular refers to the region of the TCR that interacts with a BTN3 molecule (e.g. BTN3A1) or a BTN2/BTN3 complex (e.g., BTN2A1/BTN3A1) of the disclosure, for example, the variable region of the TCR δ chain or the variable region of the TCR δ chain and TCR γ chain.

The term “epitope” in general refers to a site on an antigen, typically a (poly-) peptide, which a binding domain recognizes. The term “binding domain” in its broadest sense refers to an “antigen binding site”, i.e., characterizes a domain of a molecule which binds/interacts with a specific epitope on an antigenic target. An antigenic target may comprise a single epitope, or may comprise at least two epitopes, and can include any number of epitopes depending on the size, conformation, and type of antigen. The term “epitope” in general encompasses linear epitopes and conformational epitopes. Linear epitopes are contiguous epitopes comprised in the amino acid primary sequence and typically include at least 2 amino acids or more. Conformational epitopes are formed by non-contiguous amino acids juxtaposed by folding of the target antigen, and in particular target (poly-) peptide.

The term “γδ T cells” refers to cells that express γ and δ chains as part of a T-cell receptor (TCR) complex. The γδ TCR is comprised of a γ-chain and δ-chain, each containing a variable and constant Ig domain. The domains are formed by genetic recombination of variable (V), diversity (D) (for TCRδ only), joining (J), and constant (C) genes within the TCRδ and γ loci. The variable domain of each chain contains 3 solvent-exposed loops that typically contact ligand, known as the CDR1, CDR2 and CDR3 regions, the latter of which is highly diverse in composition due to the V-D-J combinatorial diversity and non-template nucleotide changes (additions and deletions) at the V-D and D-J recombination sites.

Human γδ T cells can be divided into four main populations based on TCR δ chain expression (δ1, δ2, δ3, δ5). Furthermore, the different TCR δ chains and TCR γ chains combined together to form different γδ T cell types. For example, γδ T cells expressing a TCR containing γ-chain variable region 9 (Vγ9) and δ-chain variable region 2 (Vδ2), are referred to as Vγ9Vδ2+ T cells, and these cells represent the majority of γδ T cells in peripheral blood. In humans, Vγ2, Vγ3, Vγ4, Vγ5, Vγ8, Vγ9, and Vγ11 rearrangements of the γ chain are found.

In humans, the γδ T cells can be further divided into “Vδ2” and “non-Vδ2 cells,” the latter consisting of mostly Vδ1- and rarely Vδ3- or Vδ5-chain expressing cells with Vδ4, Vδ6, Vδ7, Vδ8 also described.

γδ T cells can mediate antibody-dependent cell-mediated cytotoxicity (ADCC) and phagocytosis and can rapidly react toward pathogen-specific antigens without prior differentiation or expansion. γδ T cells respond directly to proteins and non-peptide antigens and are therefore not MHC restricted. At least some γδ T cell specific antigens display evolutionary conserved molecular patterns, found in microbial pathogens and induced self-antigens, which become upregulated by cellular stress, infections, and transformation. Such antigens are referred to herein generally as “phosphoantigens” or pAgs. γδ T cells may also respond to other antigens and ligands via TCR and (co-)receptors.

In addition, γδ T cells can be further categorized into a suite of multiple functional populations as follows: IFN-γ-producing γδ T cells, IL-17A-producing γδ T cells, antigen-presenting γδ T cells, follicular b helper γδ T cells, and regulatory γδ T cells. γδ T cells can promote immune responses exerting direct cytotoxicity, cytokine production and indirect immune responses. For example, the IFN-γ-producing phenotype is characterized by increased CD56 expression and enhanced cytolytic responses. Some γδ T cell subsets may contribute to disease progression by facilitating inflammation and/or immunosuppression. For example, IL-17A-producing γδ T cells broadly participate in inflammatory responses, having pathogenic roles during infection and autoimmune diseases.

The terms “Butyrophilins (BTNs)” and “butyrophilin like (BTNL)” molecules refer to regulators of immune responses that belong to the immunoglobulin (Ig) superfamily of transmembrane proteins. They are structurally related to the B7 family of co-stimulatory molecules and have similar immunomodulatory functions. BTNs are implicated in T cell development, activation and inhibition, as well as in the modulation of the interactions of T cells with antigen presenting cells and epithelial cells. Certain BTNs are genetically associated with autoimmune and inflammatory diseases. The human butyrophilin family includes seven members that are subdivided into three subfamilies: BTN1, BTN2 and BTN3. The BTN1 subfamily contains only the prototypic single copy BTN1A1 gene, whereas the BTN2 and BTN3 subfamilies each contain three genes BTN2A1, BTN2A2 and BTN2A3, and BTN3A1, BTN3A2 and BTN3A3, respectively. BTNL proteins share considerable homology to the BTN family members. The human genome contains four BTNL genes: BTNL2, 3, 8 and 9. The terms “Butyrophilins (BTNs)” and “butyrophilin like (BTNL)” molecules as used herein refer to isoforms of the BTNs and BTNL molecules.

Butyrophilins and BTNL molecules typically contain two Immunoglobulin-like domains: a N-terminal Ig-V-like (referred to herein as “IgV”) and a C-terminal Ig-C-like domain (referred to herein as “IgC”). BTNL2 comprises an additional Ig domain at the N-terminus. In some embodiments, the butyrophilin family proteins and BTNL molecules comprise a V-type domain and/or a B30.2 domain.

For the purposes of nomenclature only and not limitation, the amino acid sequence of a BTN2A1 is taught in NCBI RefSeq NP_008980.1, NP_510961.1, NP_001184162.1 or NP_001184163.1 and/or in SEQ ID NOs: 9 to 14. In one example, the BTN2A1 is human BTN2A1.

For the purposes of nomenclature only and not limitation, the amino acid sequence of a BTN3A1 is taught in NCBI RefSeq NP_008979.3, NP_919423.1, NP_001138480.1, NP_001138481.1, XP_005248890.1, XP_005248891.1, XP_006715046.1 and/or in SEQ ID NOs: 15 to 18. In one example, the BTN3A1 is human BTN3A1.

The terms BTN2 and BTN3 as used herein refer to any of BTN2A1, BTN2A2 and BTN2A3, and BTN3A1, BTN3A2 and BTN3A3, respectively interacting with a TCR and includes full length BTNs and TCR binding fragments or portions thereof, native BTNs as well as BTN isomers, variants, fragments and constructs.

Where not expressly stated, and unless the context indicates otherwise, the terms “BTN2” and “BTN3” also includes a TCR-binding fragment or a TCR-binding portion of any BTN2 and BTN3 disclosed herein.

A “TCR binding fragment” or “TCR binding portion” refers to any portion of a BTN2 or BTN3 molecule less than the whole that retains TCR binding.

The term “variant” as used herein refers to a BTN having substantial or significant sequence identity or similarity to a parent BTN its variable region(s) or its TCR binding region(s) and shares its biological activity, i.e., its ability to specifically bind to the TCR binding region(s) for which the parent BTN has specificity to a similar, the same or even a higher extent as the parent BTN.

The term “construct” includes proteins or polypeptides comprising at least one binding domain of, for example, a native BTN, but do not necessarily share the basic structure of a native BTN. BTN constructs and fragments are typically obtained by routine methods of genetic engineering and are often artificially constructed to comprise additional functional protein or polypeptide domains.

The term “BTN construct” also relates to fusion proteins or polypeptides comprising at least one antigen binding domain of the BTN; and one or more fusion component(s). Useful components include Ig derived hinge domains, Fc receptors; Fc domains (derived from IgA, IgD, IgG, IgE, and IgM); cytokines (such as IL-2 or IL-15); toxins; antibodies or antigen-binding fragments thereof (such as anti-CD3, anti-CD28, anti-CD5, anti-CD 16 or anti-CD56 antibodies or antigen-binding fragments thereof); CD247 (CD3-zeta), CD28, CD137, CD134 or other co-stimulatory domains; or any combinations thereof. In some embodiments, the BTN constructs include antibodies or antigen binding fragments thereof that target one or more of CD19, PSMA, GD2, PSCA, BCMA, CD123, B7-H3, CD20, CD30, CD33, CD38, CEA, CLEC12A, DLL3, EGFRvIII, EpCAM, CD307, FLT3, GPC3, gpA33, HER2, MUC16, P-cadherin, SSTR2, and mesothelin.

The term “label” or “labelling group” as used herein refers to any detectable label.

The term “position” as used herein means the position of either an amino acid within an amino acid sequence disclosed herein or the position of a nucleotide within a nucleic acid sequence disclosed herein. The term “corresponding” as used herein also includes that a position is not only determined by the number of the preceding amino acids/nucleotides but is rather to be viewed in the context of the circumjacent portion of the sequence. Accordingly, the position of a given amino acid or nucleotide in accordance with the disclosure may vary due to deletion or addition of amino acids or nucleotides elsewhere in the sequence. Thus, when a position is referred to as a “corresponding position” in accordance with the disclosure it is understood that amino acids/nucleotides may differ in terms of the specified numeral but may still have similar neighbouring amino acids/nucleotides. In order to determine whether an amino acid residue or nucleotide in a given sequence corresponds to a certain position in the amino acid or nucleotide sequence of a “parent” amino acid/nucleotide sequence, the skilled person can use means and methods well-known in the art, e.g., sequence alignments, either manually or by using computer programs.

As used herein, the term “binding” in reference to the interaction of Vδ2+ TCR or Vγ9Vδ2+ TCR to a modified BTN3 molecule (e.g., BTN3A1) or a BTN2/BTN3 complex of the disclosure means that the interaction is dependent upon the presence of a particular structure (e.g., epitope) on the BTN3 molecule or BTN2/BTN3 complex. For example, the Vδ2+ chain of the TCR may bind one or more of extracellular domains (e.g., IgV and/or IgC) of the BTN3 molecule. The Vγ9+ chain of the TCR may bind one or more of extracellular domains (e.g., IgV and/or IgC) of the BTN2 molecule.

As used herein, the term “specifically binds” means that the binding interaction between the TCR and a modified BTN3 molecule or a BTN2/BTN3 complex of the disclosure is dependent on the presence of an antigenic determinant or epitope. The binding region of the TCR preferentially binds or recognizes a specific antigenic determinant or epitope even when present in a mixture of other molecules or cells expressing same. In one example, the binding region reacts or associates more frequently, more rapidly, with greater duration and/or with greater affinity with the specific antigenic determinant or epitope than it does with alternative antigenic determinants or cells expressing same. It is also understood by reading this definition that, for example, a binding region that specifically binds to a particular antigenic determinant or epitope may or may not specifically bind to a second antigenic determinant or epitope. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. In one example, “specific binding” of the TCR to the modified BTN3 molecule or a BTN2/BTN3 complex of the disclosure means binding with an equilibrium constant (KD) of 10000 μM or less, 9000 μM or less, 8000 μM or less, 7000 μM or less, 6000 μM or less, 5000 μM or less, 4000 μM or less, 3000 μM or less, 2000 μM or less, 1000 μM or less, such as 900 μM or less, 800 μM or less, 700 μM or less, 600 μM or less, 500 μM or less, 400 μM or less, 300 μM or less, 200 μM or less, or 100 μM or less, such as 90 μM or less, such as 85 μM or less, for example 50 μM or less, such as, 45 μM or less, for example, between 10 μM and 1000 μM, 10 μM and 500 μM, 10 and 100 μM, 40 μM and 90 μM, or 45 μM and 85 μM.

As used herein, the term “enhances binding” in reference to the interaction of a TCR to i) a modified BTN3 molecule or ii) a BTN2/BTN3 complex means that the TCR reacts or associates with i) a modified BTN3 molecule or ii) a BTN2/BTN3 complex of the disclosure more frequently, more rapidly, with greater duration and/or with greater affinity than to i) the unmodified BTN3 counterpart having a glutamic acid (E) at a position that corresponds to amino acid 106 of the amino acid sequence shown in SEQ ID NO: 2, or ii) to BTN2 or BTN3 alone, respectively. In one example, “enhanced binding” to a modified BTN3 molecule or a BTN2/BTN3 complex of the disclosure or cell expressing same means that the TCR binds with an equilibrium constant (KD) of 100 μM or less, 50 μM or less, 40 μM or less, 30 μM or less, or 20 μM or less, or 10 μM or less, for example, between 10 μM and 100 μM, 20 μM and 50 μM, 30 and 50 μM, 40 μM and 50 μM, for example, about 45 μM. Binding of a TCR to a modified BTN3 molecule or a BTN2/BTN3 complex of the disclosure may induce or enhance Vδ2+ TCR activation. The TCR may induce or enhance Vδ2+Vγ9+ and/or Vδ2Vγ9−γδ TCR activation. For example, the TCR may induce or enhance Vδ2+γδ TCR activation, including but not limited to, Vδ2+Vγ9+ and/or Vδ2+Vγ1/2/3/4/5/8/10/11 γδ TCR activation. The activation may be phosphoantigen-independent or phosphoantigen-dependent. For example, without being bound by theory or motivation, binding of the TCR to a modified BTN3 or a BTN2/BTN3 complex of the disclosure may be independent of antigen (e.g., pAg) activation. Binding of the TCR to a modified BTN3 or a BTN2/BTN3 complex of the disclosure may be stimulatory for γδ T cells and may activate one or more of cytolytic function, cytokine production of one or more cytokines, or proliferation of the γδ T cells.

As used herein, the term “BTN2/BTN3 complex” refers to a complex of a BTN2 molecule and a BTN3 molecule, for example, BTN2A1 and BTN3A1 complex. The complex may be on the surface of a cell, for example, a tumor cell, monocyte, macrophage, dendritic cell, a parenchymal cell, and/or natural killer (NK) cell. Alternatively, the complex may be an isolated or soluble BTN2/BTN3 complex. The BTN2/BTN3 complex may be an heteromeric complex or a multimeric complex. The complex may comprise one or more BTN2 molecules such as BTN2A1 and/or BTN2A2 and/or BTN2A3 and/or one or more BTN3 molecules such as BTN3A1 and BTN3A2 and/or other proteins such as ATP-binding cassette transporter A1 (ABCA1). The BTN2 and/or the BTN3 molecule may be present in monomer or dimeric form.

In some embodiments, the BTN2/BTN3 complex of the disclosure comprises an heterodimerization domain. An “heterodimerization domain,” as used herein, refers to a domain formed on dimerization of a BTN2 and a BTN3 molecule. For example, the BTN2 molecule comprises a first heterologous C-terminal dimerization peptide that preferentially interacts or associates with another dimerization peptide C-terminally linked to the BTN3 molecule (second heterologous C-terminal dimerization peptide). Interaction of the dimerization peptides substantially contributes to or efficiently promotes heterodimerization (i.e., the formation of a dimer between BTN2 and BTN3, which is also referred to as a heterodimer or heteromeric complex herein). Representative heterodimerization peptides of the present disclosure include C-fos and C-jun.

In some embodiments, the BTN2/BTN3 heteromeric complex of the disclosure is expressed as a single chain fusion protein.

As used herein a “soluble BTN2/BTN3 complex” refers to a BTN2/BTN3 complex consisting of BTN chains that, minimally, do not comprise the transmembrane region of the full length BTN chains or comprise mutated BTN chains so that the BTN chains, when expressed by a cell, will not associate with the membrane. Most typically, a soluble complex will consist of only the extracellular domains of the native BTN chains (i.e., the BTN chains lack the transmembrane and cytoplasmic domains).

As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the terms “disease”, “disorder” or “condition” refers to a disruption of or interference with normal function and is not to be limited to any specific condition and will include diseases or disorders.

As used herein, a subject “at risk” of developing a disease or condition or relapse thereof or relapsing may or may not have detectable disease or symptoms of disease and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering a BTN or BTN complex, a nucleic acid, vector, cell, or composition described herein to thereby reduce or eliminate at least one symptom of a specified disease or condition or to slow progression of the disease or condition.

As used herein, the term “preventing”, “prevent” or “prevention” includes providing prophylaxis with respect to occurrence or recurrence of a specified disease or condition. An individual may be predisposed to or at risk of developing the disease or disease relapse but has not yet been diagnosed with the disease or the relapse.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as described herein. In some examples of the present disclosure, the term “effective amount” is meant an amount necessary to effect Vδ2+ TCR γδ T cell (e.g., Vγ9δ2+ TCR γδ T cell) activation. In some examples of the present disclosure, the term “effective amount” is meant an amount necessary to effect one or more of cytolytic function, cytokine production of one or more cytokines, or proliferation of γδ T cells. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g., a “dosage” range) that can be determined through routine trial and experimentation by a medical practitioner. Accordingly, this term is not to be construed to limit the disclosure to a specific quantity, for example, weight or number of binding proteins. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the antibody or antigen binding fragment thereof to elicit a desired response in the individual. A therapeutically effective amount is also one in which any toxic or detrimental effects of the BTN or BTN complex or binding fragment thereof are outweighed by the therapeutically beneficial effects.

As used herein, the term “prophylactically effective amount” shall be taken to mean a sufficient quantity to prevent or inhibit or delay the onset of one or more detectable symptoms of a disease or condition or a complication thereof.

As used herein, the term “subject” shall be taken to mean any animal including humans, for example a mammal. Exemplary subjects include but are not limited to humans and non-human primates. For example, the subject is a human.

### BTN2/BTN3 Complexes

The inventors have surprisingly demonstrated that BTN3A1 IgV domain interacts with BTN2A1 and that a BTN2A1-BTN3A1 complex and a modified BTN3A1 can react with Vγ9Vδ2⁺ TCR, facilitating γδ T cell-mediated immunity. The inventors have also surprisingly demonstrated a modified BTN2 can enhance the ability of the BTN2-BTN3 complex to interact with Vγ9Vδ2⁺ TCR.

### BTN2 and BTN3 Chains.

The disclosure provides a BTN2 and BTN3 complex. In some embodiments, the BTN2 chain comprises or consists of an amino acid sequence as shown in any one of SEQ ID NOs: 1, or 9 to 14, or 29, 33, 37, 45, 96, or 97 and the BTN3 chain comprises or consists of an amino acid sequence as shown in any one of SEQ ID NOs: 2, 3, 4, or 15 to 18, or 30, 34, 38, or 46.

A BTN2 chain comprising an amino acid sequence having at least 70% sequence identity, at least 80% sequence identity, more preferably at least 85% sequence identity, more preferably 90% or 95% sequence identity to any one of SEQ ID NOs: 1, or 9 to 14, or 29, 33, 37, 45, 96 or 97 and a BTN3 chain comprising an amino acid sequence having at least 70% sequence identity, at least 80% sequence identity, more preferably at least 85% sequence identity, more preferably 90% or 95% sequence identity to any one of SEQ ID NOs: 2, 3, 4, or 15 to 18, or 30, 34, 38, or 46 can be used provided that the BTN2/BTN3 complex retains the advantageous capabilities of the complex evaluated in the appended examples, i.e., binds to a Vγ9Vδ2⁺ TCR to a similar, the same or even a higher extent than either BTN2 or BTN3 alone.

As used herein the term “sequence identity” indicates the extent to which two (amino acid or nucleotide) sequences have identical residues at the same positions in an alignment and is often expressed as a percentage. Preferably, identity is determined over the entire length of the sequences being compared. Thus, two copies of exactly the same sequence have 100% identity, but sequences that are less highly conserved and have deletions, additions, or replacements, may have a lower degree of identity. Those skilled in the art will recognize that several algorithms are available for determining sequence identity using standard parameters, for example, Blast (Altschul et al. (1997) Nucleic Acids Res. 25:3389-3402), Blast2 (Altschul et al. (1990) J. Mol. Biol. 215:403-410), Smith-Waterman (Smith et al. (1981) J. Mol. Biol. 147:195-197) and ClustalW. Accordingly, the amino acid sequences of SEQ ID NOs: 1 and 2, can for instance serve as “subject sequence” or “reference sequence”.

The BTN2 chain and/or BTN3 chain of the BTN2/BTN3 complex of the disclosure may comprise one or more amino acid modifications. Amino acid modifications may be introduced into the Ig-V-like (referred to herein as “IgV”) or Ig-C-like domain (referred to herein as “IgC” of BTN2 and/or BTN3 and may serve to modulate properties like binding strength and specificity, post-translational processing (e.g., glycosylation), thermodynamic stability, solubility, surface expression or BTN2/BTN3 complex assembly. Amino acid modifications include, for example, deletions from, and/or insertions into, and/or substitutions of, residues within the amino acid sequences of the native BTN2 chain and/or BTN3 chain.

Exemplary substitutional variants are those including amino acid substitutions in variable region(s) of the BTN chain(s), the framework region(s) or the constant region(s). Particularly envisaged herein are conservative amino acid substitutions.

Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “non-conservative substitutions” are defined as exchanges of an amino acid by another amino acid listed in a different group of the six standard amino acid groups (1) to (6) shown above.

In some embodiments, the substitutions may also include non-classical amino acids (e.g., selenocysteine, pyrrolysine, N′-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general). Mutations may also be made to the nucleotide sequences of the heterodimeric proteins by reference to the genetic code, including taking into account codon degeneracy.

The BTN2 chain and/or BTN3 chain of the BTN2/BTN3 complex of the disclosure may further comprise an immunoglobulin constant-like (IgC) region. The constant region can be a human constant region or derived from another species, yielding a “chimeric” molecule.

One or more cysteine bonds may be added to the BTN constant region. The addition of a disulfide bond in the constant region may foster correct pairing of the BTN chains. Besides additional cysteine bridges, other useful modifications include, for instance, the addition of leucine zippers and/or ribosomal skipping sequences, for example, sequence 2A from picorna virus to increase folding, expression and/or pairing of the BTN chains.

BTN2/BTN3 complexes of the disclosure include heterodimers and multimers in which at least one BTN2 chain and at least one BTN3 chain are linked to each other. In its simplest form a multivalent BTN2/BTN3 complex according to the disclosure comprises a multimer of two or three or four or more BTN2/BTN3 complexes associated (e.g., covalently or otherwise linked) with one another, preferably via a linker molecule. In some embodiments, the BTN2/BTN3 complex may form due to interaction of BTN2 dimer (e.g., BTN2A1 dimer) with a BTN3 dimer (e.g., a BTN3A1 dimer).

Suitable linker molecules include, but are not limited to, multivalent attachment molecules such as avidin, streptavidin, neutravidin and extravidin, each of which has four binding sites for biotin. Thus, biotinylated BTN2/BTN3 complexes can be formed into multimers having a plurality of TCR binding sites. The number of BTN2/BTN3 complexes in the multimer will depend upon the quantity of BTN2/BTN3 complexes in relation to the quantity of linker molecule used to make the multimers, and also on the presence or absence of any other biotinylated molecules. Exemplary multimers are dimeric, trimeric, tetrameric or pentameric or higher-order multimer BTN2/BTN3 complexes. Multimers of the disclosure may also comprise further functional entities such as labels or drugs or (solid) carriers.

BTN2/BTN3 complexes of the disclosure may be linked via a suitable linker to a spheric body, preferably a uniform bead, more preferably a polystyrene bead, most preferably a bio-compatible polystyrene bead. A pre-defined fluorescence dye may be incorporated into the bead.

BTN2/BTN3 complexes of the disclosure may be fused to one or more fusion component(s) including antibodies and antibody fragments. Exemplary antibody fragments that can be used include fragments of full-length antibodies, such as (s)dAb, Fv, Fd, Fab, Fab′, F(ab′)2 or “r IgG” (“half antibody”); modified antibody fragments such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies, multibodies such as triabodies or tetrabodies, and single domain antibodies such as nanobodies or single variable domain antibodies comprising only one variable domain, which might be VHH, VH or VL.

BTN2/BTN3 complexes of the invention may be fused to one or more antibody or antibody fragments, yielding monovalent, bivalent and polyvalent/multivalent constructs and thus monospecific constructs, specifically binding to only one target antigen as well as bispecific and polyspecific/multispecific constructs, which specifically bind more than one target antigen, for example, two, three or more, through distinct antigen binding sites.

Optionally, a linker may be introduced between the one or more of the domains or regions of the BTN2/BTN3 complexes of the disclosure and/or the one or more fusion component(s) described herein. Linkers are known in the art. In general, linkers include flexible, cleavable and rigid linkers and will be selected depending on the type of construct and intended use/application. For example, for therapeutic application, non-immunogenic, flexible linkers are often preferred in order to ensure a certain degree of flexibility or interaction between the domains while reducing the risk of adverse immunogenic reactions. Such linkers are generally composed of small, non-polar (e.g., Gly) or polar (e.g., Ser or Thr) amino acids and include “GS” linkers consisting of stretches of Gly and Ser residues. Linkers may also be for example synthetic (e.g., PEG), or a hinge region of an antibody (e.g., of IgG, IgA, IgD, and IgE, inclusive of subclasses (e.g., IgG1, IgG2, IgG3, and IgG4, and IgA1 and IgA2)), or may comprise an Fc domain of an antibody (e.g., of IgG, IgA, IgD, and IgE, inclusive of subclasses (e.g., IgG1, IgG2, IgG3, and IgG4, and IgA1 and IgA2)).

Particularly useful BTN2/BTN3 constructs are those comprising at least one BTN2 chain and at least one BTN3 chain, optionally linked to each other and fused, optionally via a liker, to at least one antibody or an antibody fragment (such as a single chain antibody fragment (scFv)) directed against a surface antigen or epitope. Said construct can in general have any structure as long the “BTN portion” retains its ability to recognize the antigenic target defined herein, and the “antibody portion” binds to the desired surface antigen or epitope, thereby recruiting and targeting the BTN2/BTN3 complex or cell expressing same to the target cell. Such constructs may advantageously serve as “adapters” joining an antigen presenting cell and a lymphocyte (such as a cytotoxic T cell or NK cell) together. An example of such a fusion protein is a construct engineered according to the principle of a bi-specific T-cell engager (BiTE®) consisting of two single-chain variable fragments (scFvs) of different antibodies, on a single peptide chain of about 55 kilodaltons (kD). Accordingly, a BTN2/BTN3 construct of the disclosure may comprise at least one TCR antigen binding domain as described herein (for example, a BTN2A1 chain and BTN3A chain complexed to each other) linked to a scFv (or other binding domain) of the desired binding specificity, for example, CD3 or CD56. The scFv (or other binding domain) binds to CD3 for T cells activation or to CD56 for NK cell activation. Also envisaged herein are tribodies comprising at least one TCR antigen binding domain as described herein, an scFv (or other binding domain) and a further domain for targeting the construct to, for example, a site of action within the body (e.g., an Fc domain).

The BTN2/BTN3 complexes of the disclosure may be provided in “isolated” or “substantially pure” form. “Isolated” or “substantially pure” when used herein means that the BTN2 and/or BTN3 have been isolated and/or recovered from a component of its production environment, such that the “isolated” BTN2/BTN3 complex is free or substantially free of other contaminant components from its production environment that might interfere with its therapeutic or diagnostic use. Contaminant components may include enzymes, hormones, and other proteinaceous or non-proteinaceous solutes. “Isolated” BTN2/BTN3 complexes will thus be prepared by at least one purification step removing or substantially removing these contaminant components. The aforementioned definition is equally applicable to “isolated” polynucleotides/nucleic acids, mutatis mutandis.

The BTN2/BTN3 complexes of the disclosure may comprise one or more additional modifications as described below. The modifications described below will typically be covalent modifications and can be accomplished using standard techniques known in the art. In some circumstances, amino acid modifications in the BTN2/BTN3 complexes may be required in order to facilitate the introduction of said modifications.

The BTN2/BTN3 complexes, in particular soluble BTN2/BTN3 complexes of the disclosure can be labelled. Useful labels are known in the art and can be coupled to the BTN2 chain or BTN3 chain, or to the BTN2/BTN3 complex using routine methods, optionally via linkers of various lengths. In general, labels fall into a variety of classes, depending on the assay in which they are to be detected—the following examples include, but are not limited to: isotopic labels, which may be radioactive or heavy isotopes, such as radioisotopes or radionuclides (e.g., 3H, 14C, 15N, 35S, 89Zr, 90Y, 99Tc, 111In, 125I, 131 I); magnetic labels (e.g., magnetic particles); redox active moieties; optical dyes (including, but not limited to, chromophores, phosphors and fluorophores) such as fluorescent groups (e.g., FITC, rhodamine, lanthanide phosphors), chemiluminescent groups, and fluorophores which can be either small molecule fluorophores or proteinaceous fluorophores; enzymatic groups (e.g. horseradish peroxidase, β-galactosidase, luciferase, alkaline phosphatase; biotinylated groups; or predetermined polypeptide epitopes recognized by a secondary reporter (e.g., leucine zipper pair sequences, binding sites for secondary antibodies, metal binding domains, epitope tags, etc.). Labelling is particularly envisaged when the BTN2/BTN3 complexes or especially soluble BTN2/BTN3 complexes constructs are intended for diagnostic use.

The BTN2/BTN3 complexes in particular soluble BTN2/BTN3 complexes of the disclosure can be modified by attaching further functional moieties, for example, for reducing immunogenicity, increasing hydrodynamic size (size in solution), solubility and/or stability (e.g., by enhanced protection to proteolytic degradation) and/or extending serum half-life.

Exemplary functional moieties for use in accordance with the disclosure include peptides or protein domains binding to other proteins in the human body (such as serum albumin, the immunoglobulin Fc (IgFc) region or the neonatal Fc receptor (FcRn polypeptide chains of varying length (e.g., XTEN technology or PASylation®), non-proteinaceous polymers, including, but not limited to, various polyols such as polyethylene glycol (PEGylation), polypropylene glycol, polyoxyalkylenes, or copolymers of polyethylene glycol and polypropylene glycol, or of carbohydrates, such as hydroxyethyl starch (e.g., HESylation®) or polysialic acid (e.g., PolyXen® technology). In some embodiments, the BTN2 chain and/or BTN3 of the BTN2/BTN3 complexes of the disclosure are fused to human serum albumin or IgFc or modified variants thereof having altered binding affinity for FcRn.

Other useful functional moieties include “suicide” or “safety switches” that can be used to shut off host cells comprising a BTN2/BTN3 complexes of the disclosure in a patient's body. An example is the inducible Caspase 9 (iCasp9) “safety switch”. Briefly, host cells are modified by well-known methods to express a Caspase 9 domain whose dimerization depends on a small molecule dimerizer drug such as AP1903/CIP, and results in rapid induction of apoptosis in the modified cells. Examples for other “suicide” or “safety switches” are known in the art, for example, Herpes Simplex Virus thymidine kinase (HSV-TK), expression of CD20 and subsequent depletion using anti-CD20 antibody or myc tags.

BTN2/BTN3 complexes with post translation modifications such as altered phosphorylation, glycosylation, ubiquitination, nitrosylation, methylation, acetylation, lipidation pattern are also envisaged herein. As is known in the art, glycosylation patterns can depend on the amino acid sequence (e.g., the presence or absence of particular glycosylation amino acid residues, discussed below) and/or the host cell or organism in which the protein is produced. Glycosylation of polypeptides is typically either N-linked or O-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of or substitution by, one or more serine or threonine residues to the starting sequence.

Another means of glycosylation of BTN2/BTN3 complexes is by chemical or enzymatic coupling of glycosides to the protein. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. Similarly, deglycosylation (i.e., removal of carbohydrate moieties present on the binding molecule) may be accomplished chemically, for example, by exposing the TCRs to trifluoromethanesulfonic acid, or enzymatically by employing endo- and exo-glycosidases.

It is also conceivable to add a drug such as a small molecule compound to the BTN2/BTN3 complexes, in particular soluble BTN2/BTN3 complexes, of the disclosure. Linkage can be achieved via covalent bonds, or non-covalent interactions such as through electrostatic forces. Various linkers, known in the art, can be employed in order to form the drug conjugates. Such complexes could target a cytotoxic drug (e.g., PBDs) to Vδ2⁺ T cells in order to specifically kill this T cell subset in a treatment of, for example, an autoimmune disease, transplantation rejection, graft versus host disease, or graft versus tumour effect.

The BTN2/BTN3 complexes, in particular soluble BTN2/BTN3 complexes, of the disclosure can be modified to introduce additional domains which aid in identification, tracking, purification and/or isolation of the respective molecule (tags). Non-limiting examples of such tags comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. Strep II-tag), His-tag, CD20, Her2/neu tags, myc-tag, FLAG-tag, T7-tag, SpyCatcher or GFP-tags, or other fluorescent or luminescent tags known in the art.

Epitope tags are useful examples of tags that can be incorporated into the BTN2/BTN3 complexes of the disclosure. Epitope tags are short stretches of amino acids that allow for binding of a specific antibody and therefore enable identification and tracking of the binding and movement of soluble TCRs or host cells within the patient's body or cultivated host cells. Detection of the epitope tag, and hence, the tagged BTN2/BTN3 complexes, can be achieved using a number of different techniques. Examples of such techniques include: immunohistochemistry, immunoprecipitation, flow cytometry, immunofluorescence microscopy, ELISA, immunoblotting (“Western”), and affinity chromatography. The epitope tags can for instance have a length of 6 to 15 amino acids, in particular 9 to 11 amino acids. It is also possible to include more than one epitope tag in the TCR of the disclosure.

Tags can further be employed for stimulation and expansion of host cells comprising a BTN2/BTN3 complexes of the disclosure by cultivating the cells in the presence of binding molecules (antibodies) specific for said tag.

### Modified BTN2

The present disclosure also provides a modified BTN2. In preferred embodiments, the modified BTN2 is complexed to a BTN3 molecule. The modified BTN2 of the disclosure comprises a modification (e.g., substitution, deletion, or insertion) at a position that corresponds to serine (S) 44 of the amino acid sequence shown in SEQ ID NO: 1, wherein the modification enhances binding of the BTN2-BTN3 complex to a Vγ9Vδ2⁺ TCR. In one example, the BTN2 of the disclosure comprises an arginine at a position that corresponds to serine (S) 44 of the amino acid sequence shown in SEQ ID NO: 1. The skilled person will appreciate that the description of BTN2 variants, constructs, and fragments provided above in the context of the BTN2/BTN3 complex of the disclosure also applies to modified BTN2 molecules of the disclosure.

The aforementioned description of the BTN2 chain in the context of the BTN2/BTN3 complex of the disclosure is equally applicable to modified BTN2 molecules of the disclosure, mutatis mutandis. It is intended that variants, constructs, and fragments of the modified BTN2 molecules be covered, as well as fusions, dimers, multimers thereof, and soluble forms.

### Modified BTN3

The present disclosure also provides a modified BTN3 that can be used alone or complexed to a BTN2 molecule. The modified BTN3 of the disclosure comprises a modification (e.g., substitution, deletion, or insertion) at a position that corresponds to glutamic acid (E) 106 of the amino acid sequence shown in SEQ ID NO: 2, wherein the modification enhances binding of the BTN3 to a Vδ2⁺ TCR. In one example, the BTN3 of the disclosure comprises an alanine at a position that corresponds to glutamic acid (E) 106 of the amino acid sequence shown in SEQ ID NO: 2. The skilled person will appreciate that the description of BTN3 variants, constructs, and fragments provided above in the context of the BTN2/BTN3 complex of the disclosure also applies to modified BTN3 molecules of the disclosure.

The aforementioned description of the BTN3 chain in the context of the BTN2/BTN3 complex of the disclosure is equally applicable to modified BTN3 molecules of the disclosure, mutatis mutandis. It is intended that variants, constructs, and fragments of the modified BTN3 molecules be covered, as well as fusions, dimers, multimers thereof, and soluble forms.

### Heterodimerization Domain

In some embodiments, the heterodimerisation domain of the BTN2/BTN3 complexes according to the disclosure is a so-called “coiled coil” or “leucine zipper”. These terms are used to describe pairs of helical peptides which interact with each other in a specific fashion to form a heterodimer. The interaction occurs because there are complementary hydrophobic residues along one side of each zipper peptide. The nature of the peptides is such that the formation of heterodimers is very much more favourable than the formation of homodimers of the helices.

Coiled coils are common domains in many transcription factors. Examples of such structures are the leucine zippers of the GCN4 transcription factor (Kouzarides, T. and Ziff, E. “Leucine zippers of fos, jun and GCN4 dictate dimerization specificity and thereby control DNA binding”, Nature 340, 568-571 (1989). https://doi.org/10.1038/340568a0). Other basic leucine zippers include TF6, CREB1, C/EBPα, Fos, or Jun, viral fusion proteins influenza hemagglutinin or HIV gp41, or other coiled coil domains APC or ProP. A compilation of known coiled coil dimerization domains is provided at http://coiledcoils.chm.bris.ac.uk/ccplus/search/and http://www.chm.bris.ac.uk/org/woolfson/oli.html. These coiled coils are also described in Testa O D, Moutevelis E, and Woolfson D N, 2009, “CC+: a relational database of coiled-coil structure”, Nucleic Acid Research, Vol. 37, Database issue, D315-D322; and Moutevelis E and Woolfson, D N, 2009, “A periodic table of coiled-coil protein structures”, Journal of Molecular Biology 385(3): 726-732.

Leucine zippers may be synthetic or naturally occurring. Synthetic leucines can be designed to have a much higher binding affinity than naturally occurring leucine zippers, which is not necessarily an advantage. In fact, preferred leucine zippers for use in the disclosure are naturally occurring leucine zippers or leucine zippers with a similar binding affinity. Leucine zippers from the c-jun and c-fos protein are an example of leucine zippers with a suitable binding affinity. Other suitable leucine zippers include those from the myc and max proteins (B. Amati et al. Nature 359(6394), 423-6 (1992)). Other leucine zippers with suitable properties could easily be designed (E. K. O'Shea et al. Curr. Biol. 3, 658-667 (1993)). For example, those with acidic and basic amino acid moieties, for example glutamic acid and arginine.

It is preferred that the BTN2/BTN3 complexes of the disclosure have approximately 40 amino acid leucine zipper fusions corresponding to the heterodimerisation domains from c-jun (achain) and c-fos (pchain) (E. K. O'Shea et al. Science 245(4918), 646-8 (1989); E. K. O'Shea et al. Cell 68(4), 699-708 (1992); J. N Glover and S. C. Harrison Nature 373(6511), 257-61(1995)).

Longer leucine zippers may be used. Since heterodimerisation specificity appears to be retained even in quite short fragments of some leucine zipper domains (E. K. O'Shea et al. Cell 68(4), 699-708 (1992)), it is possible that a similar benefit could be obtained with shorter c-jun and c-fos fragments. Such shorter fragments could have as few as 8 amino acids for example. Thus, the leucine zipper domains may be in the range of 8 to 60 amino acids long.

The molecular principles of specificity in leucine zipper pairing is well characterised (W. H. Landschulz et al. Science 240(4860), 175964 (1988)) and leucine zippers can be designed and engineered by those skilled in the art to form homodimers, heterodimers or trimeric complexes (K. J. Lumb and P. S. Kim Biochemistrv 34(27), 8642-8 (1995); S. Nautiyal et al. Biochemistry 34(37), 11645-51 (1995); J. A. Boice et al. Biochemistrv 35(46), 14480-5 (1996); H. Chao et al. Biochemistrv 35(37), 12175-85 (1996)). Designed leucine zippers, or other heterodimerisation domains, of higher affinity than the c-jun and c-fos leucine zippers may be beneficial for the expression of BTN2-BTN3 complexes in some systems. However, as mentioned in more detail below, when BTN2-BTN3 complexes are folded in vitro, a solubilising agent is preferably included in the folding buffer to reduce the formation of unproductive protein aggregates.

One interpretation of this phenomenon is that the kinetics of folding of the leucine zipper domains are faster than for the BTN2 and BTN3 chains, leading to dimerisation of unfolded BTN2 and BTN3 chains, in turn causing protein aggregation. By slowing the folding process and inhibiting aggregation by inclusion of solubilising agent, the protein can be maintained in solution until folding of both fusion domains is completed. Therefore, heterodimerisation domains of higher affinity than the c-fos and c-jun leucine zippers may require higher concentrations of solubilising agent to achieve a yield of BTN2-BTN3 complexes comparable to that for c-jun and c-fos.

Different biological systems use a variety of methods to form stable homo and hetero protein dimers, and each of these methods in principle provide an option for engineering dimerisation domains into genetically modified proteins. Leucine zippers (T. Kouzarides and E. Ziff Nature 340(6234), 568-71 (1989)) are probably the most popular dimerisation modules and have been widely used for production of genetically designed dimeric proteins. Thus, the leucine zipper of GCN4, a transcriptional activator protein from the yeast Saccharomyces cerevisiae, has been used to direct homodimerisation of a number of heterologous proteins (J. C. Hu et al. Protein Sci. 2(7), 1072-84 (1993); N. J. Greenfield et al. Biochemistrv 37(21), 7834-43 (1998)). The preferred strategy is to use zippers that direct formation of heterodimeric complexes such as the Jun/Fos leucine zipper pair (J. de Kruif and T. Logtenberg J. Biol. Chem. 271(13), 7630-4 (1996); L. G Riley et al. [published erratum appears in Protein Eng. 9(9), 831 (1996)] Protein Eng. 9(2), 223-30 (1996)).

The heterodimerisation domain of the BTN2/BTN3 complexes of the disclosure is not limited to leucine zippers. Thus, it may be provided by disulphide bridge-forming elements. Alternatively, it may be provided by the SH3 domains and hydrophobic/proline rich counter domains, which are responsible for the protein-protein interactions seen among proteins involved in signal transduction (reviewed by Schlessinger (J. Schlessinger, Curr. Opin. Genet. Dev. 4(1), 25-30 (1994)). Other natural protein-protein interactions found among proteins participating in signal transduction cascades rely on associations between post-translationally modified amino acids and protein modules that specifically recognise such modified residues.

Such post-translationally modified amino acids and protein modules may form the heterodimerisation domain of the BTN2-BTN31 complexes of the disclosure. An example of a protein pair of this type is provided by tyrosine phosphorylated receptors such as Epidermal Growth Factor Receptor or Platelet Derived Growth Factor Receptor and the SH2 domain of GRB2 (E. J. Lowenstein et aL. Cell 70(3), 431-42 (1992); L. Buday and J. Downward. Cell 73(3), 611-20 (1993)). Methods for engineering completely artificial modules have also been developed (Z. A. Zhang et al. Curr. Biol. 9(8), 417-20 (1999)).

The heterodimerisation domain may be formed from binding portions of nuclear receptors, estrogen receptors, androgen receptors, glucocorticoid receptors, basic helix-loop-helix MyoD or c-Myc, helix-turn-helix LuxR, TetR, or cl, or transmembrane regions of integrin α and β subunits, glycophorin A, tyrosine kinases, or GPCRs; or G protein βγ complexes from heterotrimeric G protein complexes, TIM, ADH5, 14-3-3 proteins or their binding partners Bad or Bax; or portions of any other protein dimers.

In some embodiments, the heterodimerization domain comprises an Fc domain of an antibody. In some embodiments, heterodimerisation domain comprises an Fc domain of an antibody (e.g., of IgG, IgA, IgD, and IgE, inclusive of subclasses (e.g., IgG1, IgG2, IgG3, and IgG4, and IgA1 and IgA2) or variants thereof to, for example, improve in vivo half life.

In some embodiments, the heterodimerization domain uses an heterodimeric Fc variant (the so-called “Knobs-into-Holes (KiH)” Fc). A “knob” may be introduced into one CH3 domain (CH3A) by substitution of a small residue with a bulky one (e.g., T366WCH3A in EU numbering). To accommodate the “knob,” a complementary “hole” surface is created on the other CH3 domain (CH3B) by replacing the closest neighboring residues to the knob with smaller ones (e.g., T366S/L368A/Y407VCH3B). The “hole” mutation can be optimized by structured-guided phage library screening. Heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively. The KiH Fc can be further engineered to improve purity and stability by introducing an additional inter-CH3 domain disulfide bond pair, for example, S354CCH3A-Y349CCH3B, generating the KiHS-S Fc variant. This may increase the yield of heterodimerization (˜95%) and improved thermal melting (Tm) of the CH3 domain by 6 to ˜78° C.

The heterodimerization may also be formed by binding molecules such as antibodies or nanobodies which have affinity for C-terminal portion of the first and second BTN2 and BTN3 polypeptide chains. Antibodies and nanobodies can form both homo- and heterodimers depending on the specificity of the antigen binding regions. Preferably a bispecific binding molecule is used to form a heterodimer. Alternatively, two antibodies having differing specificity for the first or second polypeptide can be used and may be linked, for example by a secondary antibody (M. Spaargaren et al., Antibody-induced dimerization activates the epidermal growth factor receptor tyrosine kinase, J. Biol. Chem. 266:1733-9 (1991)).

Glutathione S-Transferase (GST) can also be used to form dimers. GST can be added to the C-terminal ends of a protein to drive dimerization with other GST tagged proteins (Y. Maru et al., The Dimerization Property of Glutathione S-Transferase Partially Reactivates Bcr-Abl Lacking the Oligomerization Domain, J. Biol. Chem. 271(26):15353-15357 (1996)). However, GST-tagged polypeptides will form homodimers or heterodimers in a stochastic manner, rather than preferentially forming heterodimers.

Rapamycin, which comprises a FKBP binding portion and an FRB binding portion, can act as an intermediate to form heterodimers. The BTN and BTN3 polypeptides can be fused to FKBP and FRB to form fusion proteins which are then dimerized by rapamycin (V. M. Rivera et al., A humanized system for pharmacologic control of gene expression. Nat. Med. 2:1028-32 (1996)).

Gibberellin is a family of over 100 related plant hormones which bind to their cognate receptors. Once bound, the receptor changes configuration permitting interaction with a secondary receptor. Accordingly, to form a heterodimer, the one subunit of the heteromeric complex is bound to the hormone receptor with the other subunit bound to the secondary receptor. Subsequently Gibberellin is added inducing dimerization of the two subunits into a heterodimer (T. Miyamoto et al., Rapid and orthogonal logic gating with a gibberellin-induced dimerization system, Nat. Chem. Biol. 8:465-470 (2012). Similarly, the plant hormone Abscisic acid can be used in a similar way (K. I Miyazono et al., Structural basis of abscisic acid signalling, Nature. 462:609-14 (2009)).

FK506, which interacts with FKBR, may be fused with dexamethasone or cyclosporin-A to form a dimerization intermediate (P. J. Belshaw et al., Controlling protein association and subcellular localization with a synthetic ligand that induces heterodimerization of proteins, Proc. Natl. Acad. Sci. U.S.A. 93: 4604-7 (1996). In the present context, and in relation to FKBR fused with cyclosporin-A) one subunit in the heteromeric complex would include a C-terminal FKBR as a dimerization peptide while the other dimerization peptide includes C-terminal CyclophilinA. A heterodimer is then formed by exposing the two subunits to FKBR-cyclosporin-A. FKBR fused with dexamethasone may be used in the same way but with glucocorticoid receptor (which interacts with dexamethasone) as the dimerization peptide on the second subunit.

Semi-covalent inducible chemical dimerization can be induced by the SNAP-tag ligand 06-benzylguanine (BG) combined with methotrexate which forms a covalent attachment with the SNAP-tag binding HAGT and a non-covalent interaction with the methotrexate binding DHFR (S. Gendreizig et al., Induced protein dimerization in vivo through covalent labelling, J. Am. Chem. Soc. 125:14970-14971 (2003)). The first subunit of the heteromeric complex would include HAGT with the second subunit including DHFR. These two subunits would then be dimerized by the inclusion of the SNAP-tag. CLIP-tag, which is a modified version of SNAP-tag, is engineered to react with benzylcytosine derivatives instead of BG and can be used in a comparable way to NSAP-Tag to dimerize proteins (Erdmann et al., Labeling Strategies Matter for Super-Resolution Microscopy: A Comparison between HaloTags and SNAP-tags. Cell Chemical Biology (2019)).

An alternative technique for inducible dimerization is light induced dimerization. These techniques include the photoreactive chemical dimerizes pRap, cRB-A, PhAP and MeNV-HaXS (A. V. Karginov et al., Light Regulation of Protein Dimerization and Kinase Activity in Living Cells Using Photocaged Rapamycin and Engineered FKBP. J. Am. Chem. Soc. 420-423 (2010); N. T. Umeda et al., A photocleavable rapamycin conjugate for spatiotemporal control of small GTPase activity. J. Am. Chem. Soc. 133:12-4 (2011); S. J. Ahmed et al., Photocleavable dimerizer for the rapid reversal of molecular trap antagonists. J. Biol. Chem. 289:4546-52 (2014); and D. Erhart et al., Chemical development of intracellular protein heterodimerizers. Chem. Biol. 20:549-57 (2013)). These light-inducible dimerization molecules function by binding with a dimerization domain only after exposure to specific wavelengths of light. The subsequent interaction with the dimerization domain causes a confirmational change which allows for coupling with a binding partner thereby dimerizing any linked peptides.

Oligomerization of multiple polypeptides may also be achieved by cross-linking non-canonical amino acids (I. Coin, Application of non-canonical crosslinking amino acids to study protein-protein interactions in live cells, Curr. Op. in Chem. Biol. 46:156-163 (2018). Non-canonical amino acids which carry chemical groups which are not present in the 20 canonical amino acids can be incorporated at the C-terminal of the first and second subunits of the heteromeric complex, for example by way of techniques such as amber stop codon suppression. Chemical groups such as benzophenones, diazirines and aromatic azides carried on the non-canonical amino acids can then be cross-linked by way of light irradiation at specific wavelength resulting in C-terminal dimerization (J. E. Hoffmann, Bifunctional Non-Canonical Amino Acids: Combining Photo-Crosslinking with Click Chemistry. Biomolecules. 10:578 (2020)).

“Click-chemistry” can alternatively be used to prepare dimerized polypeptides. The first and second peptide can be synthesised with an azidohexanoic acid on the C-terminus of one polypeptide and an aza-dibenzocyclooctyne (DIBAC) on the C-terminus of the other polypeptide. These two polypeptides are then dimerized by azido-modified ubiquitin and ubiquitin equipped with a cyclooctyne (M. D. Witte et al., Preparation of unnatural N-to-N and C-to-C protein fusions, Proc. Nat. Acad. Sci. 109(30):11993-11998 (2012)).

In some embodiments, the heteromeric complex comprises at least a trimer including at least as homodimer paired with heteromeric monomer subunit. Alternatively, two homodimers can be paired to form a tetramer.

Foldon is an example of a trimerization domain which can be used with the present invention. Foldon consists of a 27 amino acid domain which constitutes the C-terminal end of the fibritin protein from bacteriophage T4. This domain is the oligomerisation domain of T4 fibritin and has been shown to oligomerise monomer peptides into trimers (S. Guthe et al., Very fast folding and association of a trimerization domain from bacteriophage T4 fibritin, J. Mol. Biol., 337(4):905-915, (2004)). Refolding of the BTN2 and BTN3 chains of the complex occurs in vitro under suitable refolding conditions. In a particular embodiment, a recombinant BTN2/BTN3 complex with correct conformation is achieved by refolding solubilised BTN2 and BTN3 chains in a refolding buffer comprising a solubilising agent, for example urea. Advantageously, the urea may be present at a concentration of at least 0.1 M or at least 1 M or at least 2.5M, or about 5M. An alternative solubilising agent which may be used is guanidine, at a concentration of between 0.1 M and 8M, preferably at least 1 M or at least 2.5M. Prior to refolding, a reducing agent may be used to ensure complete reduction of cysteine residues. Further denaturing agents such as DTT and guanidine may be used as necessary. Different denaturants and reducing agents may be used prior to the refolding step (e.g., urea, β-mercaptoethanol). Alternative redox couples may be used during refolding, such as a cystamine/cystearnine redox couple, DTT or β-mercaptoethanol/atmospheric oxygen, oxidized and reduced glutathione, and cysteine in reduced and oxidised forms.

### Nucleic Acid

The present disclosure further provides nucleic acids encoding the BTNs or variants or fragments thereof, or BTN constructs, or BTN complexes, or single chain fusion polypeptides described herein. For example, polynucleotides encoding BTN2 (e.g., BTN2A1) or BTN3 (e.g., BTN3A1) chains, as well as BTN variants, constructs, and fragments thereof.

The term “polynucleotide” or “nucleic acid” as used herein comprises a sequence of polyribonucleotides and polydeoxribonucleotides, for example, modified or unmodified RNA or DNA, each in single-stranded and/or double-stranded form, linear or circular, or mixtures thereof, including hybrid molecules. The nucleic acids according to this disclosure thus comprise DNA (such as dsDNA, ssDNA, cDNA), RNA (such as dsRNA, ssRNA, mRNA, VfRNA), combinations thereof or derivatives (such as PNA) thereof.

A polynucleotide may comprise a conventional phosphodiester bond or a non-conventional bond (e.g., an amide bond, such as found in peptide nucleic acids (PNA)). The polynucleotides of the disclosure may also comprise one or more modified bases, such as, for example, tritylated bases and unusual bases such as inosine. Other modifications, including chemical, enzymatic, or metabolic modifications, are also conceivable, as long as a binding molecule of the invention can be expressed from the polynucleotide. The polynucleotide may be provided in isolated form as defined elsewhere herein. A polynucleotide may include regulatory sequences such as transcription control elements (including promoters, enhancers, operators, repressors, and transcription termination signals), ribosome binding site, introns, or the like.

In particular, the present invention provides a polynucleotide comprising or consisting of a nucleic acid that is at least about 70%, about 80%, about 85%, about 90%, about 91%, about 92%, about 93%, about 94%, about 95%, about 96%, about 97%, about 98%, about 99%, or 100% identical to a reference polynucleotide sequence selected from the group consisting of SEQ ID Nos: 5 to 8, or 19 to 28, or 31, 32, 35, 36, 39, 40, 47, 48, 98, or 99.

The polynucleotides described above may or may not comprise additional or altered nucleotide sequences encoding, for example, altered amino acid residues, a signal peptide to direct secretion of the encoded BTN, constant region(s) or other heterologous polypeptide(s) as described herein. Such polynucleotides may thus encode fusion polypeptides, fragments, variants and other derivatives of the binding molecules described herein.

Also, the present invention includes compositions comprising one or more of the polynucleotides described above. Also provided herein are compositions comprising a first polynucleotide and second polynucleotide wherein said first polynucleotide encodes a BTN2 chain (e.g., a BTN2A1 chain) and wherein said second polynucleotide encodes a BTN3 chain (e.g., BTN3A1 chain).

The nucleic acid sequences of the present invention may be codon-optimized for optimal expression in the desired host cell, for example, a human dendritic cell, macrophage, Langerhans cell or B cell; or for expression in bacterial, yeast or insect cells that are particularly envisaged for the expression of soluble BTNs of the invention. Codon-optimization refers to the exchange in a sequence of interest of codons that are generally rare in highly expressed genes of a given species by codons that are generally frequent in highly expressed genes of such species, such codons encoding the same amino acids as the codons that are being exchanged. Selection of optimum codons thus depends on codon usage of the host genome and the presence of several desirable and undesirable sequence motifs.

### Vector

Further provided herein is a vector, comprising one or more of the polynucleotides as described herein. A “vector” is a nucleic acid molecule used as a vehicle to transfer (foreign) genetic material into a host cell where it can for instance be replicated and/or expressed.

The term “vector” encompasses, without limitation plasmids, viral vectors (including retroviral vectors, lentiviral vectors, adenoviral vectors, vaccinia virus vectors, polyoma virus vectors, and adenovirus-associated vectors (AAV)), phages, phagemids, cosmids and artificial chromosomes (including BACs and YACs). The vector itself is generally a nucleotide sequence, commonly a DNA sequence that comprises an insert (transgene) and a larger sequence that serves as the “backbone” of the vector.

Engineered vectors typically comprise an origin for autonomous replication in the host cells (if stable expression of the polynucleotide is desired), selection markers, and restriction enzyme cleavage sites (e.g., a multiple cloning site, MCS).

Vectors may additionally comprise promoters, genetic markers, reporter genes, targeting sequences, and/or protein purification tags. Suitable vectors are known to those of skill in the art and many are commercially available.

Targeting vectors can be used to integrate a polynucleotide into the host cell's chromosome by methods known in the art. Briefly, suitable means include homologous recombination or use of a hybrid recombinase that specifically targets sequences at the integration sites. Targeting vectors are typically circular and linearized before use for homologous recombination. As an alternative, the foreign polynucleotides may be DNA fragments joined by fusion or synthetically constructed DNA fragments which are then recombined into the host cell. It is also possible to use heterologous recombination which results in random or non-targeted integration.

The vector of the present disclosure can also be an expression vector. “Expression vectors” or “expression constructs” can be used for the transcription of heterologous polynucleotide sequences, for instance those encoding the TCRs of the disclosure, and translation of their mRNA in a suitable host cell. This process is also referred to as “expression” of the TCRs of the disclosure herein. Besides an origin of replication, selection markers, and restriction enzyme cleavage sites, expression vectors typically include one or more regulatory sequences operably linked to the heterologous polynucleotide to be expressed.

The term “regulatory sequence” refers to a nucleic acid sequence necessary for the expression of an operably linked coding sequence of a (heterologous) polynucleotide in a particular host organism or host cell and thus include transcriptional and translational regulatory sequences. Typically, regulatory sequences required for expression of heterologous polynucleotide sequences in prokaryotes include a promoter(s), optionally operator sequence(s), and ribosome binding site(s). In eukaryotes, promoters, polyadenylation signals, enhancers and optionally splice signals are typically required. Moreover, specific initiation and secretory signals also may be introduced into the vector in order to allow for secretion of the polypeptide of interest into the culture medium.

A nucleic acid is “operably linked” when it is placed into a functional relationship with another nucleic acid sequence, in particular on the same polynucleotide molecule. For example, a promoter is operably linked with a coding sequence of a heterologous gene when it is capable of effecting the expression of that coding sequence. The promoter is typically placed upstream of the gene encoding the polypeptide of interest and regulates the expression of said gene.

Exemplary regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. The expression vectors may also include origins of replication and selectable markers.

Suitable selection markers for use with eukaryotic host cells include, without limitation, the herpes simplex virus thymidine kinase (tk), hypoxanthine-guanine phosphoribosyltransferase (hgprt), and adenine phosphoribosyltransferase (aprt) genes. Other genes include dhfr (methotrexate resistance), gpt (mycophenolic acid resistance) neo (G-418 resistance) and hygro (hygromycin resistance). Vector amplification can be used to increase expression levels. In general, the selection marker gene can either be directly linked to the polynucleotide sequences to be expressed or introduced into the same host cell by co-transformation.

In view of the above, the present disclosure thus further provides one or more of the nucleotide sequences described herein inserted into (i.e. comprised by) a vector. Specifically, the invention provides (replicable) vectors comprising a nucleotide sequence encoding a BTN or variant or fragment thereof, or BTN construct, or BTN complex, or single chain fusion polypeptide of the disclosure, or a BTN2 (e.g., BTN2A1) or BTN3 chain (e.g., BTN3A1 chain) thereof operably linked to a promoter.

The skilled person will readily be able to select a suitable expression vector based on, for example, the host cell intended for BTN expression. Examples for suitable expression vectors are viral vectors, such as retroviral vectors, for example, MP71 vectors or retroviral SIN vectors; and lentiviral vectors or lentiviral SIN vectors. Viral vectors comprising polynucleotides encoding the BTNs of the disclosure are for instance capable of infecting lymphocytes, which are envisaged to subsequently express the heterologous TCR. Another example for a suitable expression vector is the Sleeping Beauty (SB) transposon transposase DNA plasmid system, SB DNA plasmid. The nucleic acids and/or in particular expression constructs of the disclosure can also be transferred into cells by transient RNA transfection. For example, the viral vector may link the BTN2 and BTN3 chain genes in one vector with either an internal ribosomal entry site (IRES) sequence or a self-cleaving peptide (e.g. the 2A peptide sequence derived from a porcine tsechovirus), resulting in the expression of a single messenger RNA (mRNA) molecule under the control of the viral promoter within the transduced cell.

### Host Cell

The present disclosure further provides a host cell comprising the TCR, nucleic acid or the vector described herein.

A variety of host cells can be used in accordance with the disclosure. As used herein, the term “host cell” encompasses cells which can be or has/have been recipients of polynucleotides or vectors described herein and/or express (and optionally secrete) the BTNs or variants or fragments thereof, or BTN constructs, or BTN complexes, or single chain fusion polypeptides of the present disclosure.

The terms “cell” and “cell culture” are used interchangeably to denote the source of a BTN unless it is clearly specified otherwise. The term “host cell” also includes “host cell lines”.

In general, the term “host cell” includes prokaryotic or eukaryotic cells, and also includes without limitation bacteria, yeast cells, fungi cells, plant cells, and animal cells such as insect cells and mammalian cells, for example, murine, rat, macaque or human cells.

In view of the above, the disclosure thus provides, inter alia, host cells comprising a polynucleotide or a vector, for example, an expression vector comprising a nucleotide sequence encoding a BTN or variant or fragment thereof, or BTN construct, or BTN single chain fusion polypeptide as described herein.

Polynucleotides and/or vectors of the disclosure can be introduced into the host cells using routine methods known in the art, for example, by transfection, transformation, or the like.

“Transfection” is the process of deliberately introducing nucleic acid molecules or polynucleotides (including vectors) into target cells. An example is RNA transfection, i.e., the process of introducing RNA (such as in vitro transcribed RNA, ivtRNA) into a host cell. The term is mostly used for non-viral methods in eukaryotic cells.

The term “transduction” is often used to describe virus-mediated transfer of nucleic acid molecules or polynucleotides.

Transfection of animal cells typically involves opening transient pores or “holes” in the cell membrane, to allow the uptake of material. Transfection can be carried out using calcium phosphate, by electroporation, by cell squeezing or by mixing a cationic lipid with the material to produce liposomes, which fuse with the cell membrane and deposit their cargo inside. Exemplary techniques for transfecting eukaryotic host cells include lipid vesicle mediated uptake, heat shock mediated uptake, calcium phosphate mediated transfection (calcium phosphate/DNA co-precipitation), microinjection and electroporation.

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules).

Transformation can be effected by artificial means. For transformation to happen, cells or bacteria must be in a state of competence, which might occur as a time-limited response to environmental conditions such as starvation and cell density. For prokaryotic transformation, techniques can include heat shock mediated uptake, bacterial protoplast fusion with intact cells, microinjection and electroporation. Techniques for plant transformation include Agrobacterium mediated transfer, such as by A. tumefaciens, rapidly propelled tungsten or gold microprojectiles, electroporation, microinjection and polyethylene glycol mediated uptake.

In view of the above, the present disclosure thus further provides host cells comprising at least one polynucleotide sequence and/or vector as described herein.

For expression of the BTNs or variants or fragments thereof, or BTN constructs, or BTN complexes, or single chain fusion polypeptides of the disclosure, a host cell may be chosen that modulates the expression of the inserted polynucleotide sequences, and/or modifies and processes the gene product (i.e., RNA and/or protein) as desired. Such modifications (e.g., glycosylation) and processing (e.g., cleavage) of gene products may be important for the function of the TCR. Different host cells have characteristic and specific mechanisms for the post-translational processing and modification of gene products. Appropriate cell lines or host systems can be chosen to ensure the correct modification and processing of the product. To this end, eukaryotic host cells that possess the cellular machinery for proper processing of the primary transcript, glycosylation, and phosphorylation of the gene product may be used.

It is envisaged herein to provide (a) host cells for expressing and obtaining a BTN or variant or fragment thereof, or BTN construct, or BTN complex, or single chain fusion polypeptide of the disclosure, in particular in soluble form (“production host cells”) and (b) host cells expressing a BTN or variant or fragment thereof, or BTN construct, or BTN complex, or single chain fusion polypeptide of the disclosure and having effector function (“effector host cells”). Such “effector host cells” are particularly useful for therapeutic applications and are envisaged for administration to a subject in need thereof. Preferred “effector host cells” include antigen presenting cells, for example, dendritic cells, macrophages, Langerhans cells and B cells.

### Production Host Cell

“Production host cells” used for the expression of soluble BTNs or variants or fragments thereof, BTN constructs, or BTN complexes, or BTN single chain fusion polypeptides of the disclosure are preferably capable of expressing high amounts of recombinant protein.

Exemplary mammalian host cells that can be used for as “production host cells” include Chinese Hamster Ovary (CHO cells) including DHFR minus CHO cells such as DG44 and DUXBI 1, NSO, COS (a derivative of CVI with SV40 T antigen), HEK293 (human kidney), Expi293 and SP2 (mouse myeloma) cells. Other exemplary host cell lines include, but are not limited to, HELA (human cervical carcinoma), CVI (monkey kidney line), VERY, BHK (baby hamster kidney), MDCK, 293, W138, R1610 (Chinese hamster fibroblast) BALBC/3T3 (mouse fibroblast), HAK (hamster kidney line), P3×63-Ag3.653 (mouse myeloma), BFA-IcIBPT (bovine endothelial cells), and RAJI (human lymphocyte). Host cell lines are typically available from commercial services, the American Tissue Culture Collection (ATCC) or from published literature.

Non-mammalian cells such as bacterial, yeast, insect or plant cells are also readily available and can also be used as “production host cells” as described above. Exemplary bacterial host cells include enterobacteriaceae, such Escherichia coli, Salmonella; Bacillaceae, such as Bacillus subtilis; Pneumococcus; Streptococcus, and Haemophilus influenza. Other host cells include yeast cells, such as Saccharomyces cerevisiae, and Pichia pastoris. Insect cells include, without limitation, Spodoptera frugiperda cells. In accordance with the foregoing, conceivable expressions systems (i.e., host cells comprising an expression vector as described above) include microorganisms such as bacteria (e.g., E. coli, B. subtilis) transformed with recombinant bacteriophage DNA, plasmid DNA or cosmid DNA expression vectors; yeast (e.g., Saccharomyces, Pichia) transformed with recombinant yeast expression vectors; insect cell systems infected with recombinant virus expression vectors (e.g., baculovirus); plant cell systems infected with recombinant virus expression vectors (e.g., cauliflower mosaic virus, CaMV; tobacco mosaic virus, TMV) or transformed with recombinant plasmid expression vectors (e.g., Ti plasmid). Mammalian expression systems harboring recombinant expression constructs containing promoters derived from the genome of mammalian cells (e.g., metallothionein promoter) or from mammalian viruses (e.g., the adenovirus late promoter; the vaccinia virus 7.5K promoter, the cytomegalovirus (CMV) major immediate-early promoter (MIEP) promoter) are often preferred. Suitable mammalian host cells can be selected from known cell lines (e.g., COS, CHO, BLK, 293, 3T3 cells), however it is also conceivable to use dendritic cells, macrophages, Langerhans cells and B cells.

In accordance with the foregoing, the present disclosure also provides a method for producing and obtaining a BTN or variant or fragment thereof, or BTN construct, or BTN complex, or single chain fusion polypeptide as described herein comprising the steps of (i) culturing a host cell (i.e., a production host cell) under conditions causing expression of said BTN or variant or fragment thereof, or BTN construct, or BTN complex, or single chain fusion polypeptide and (ii) purifying said BTN or variant or fragment thereof, or BTN construct, or BTN complex, or single chain fusion polypeptide. In some embodiments, the methods also comprise the step of refolding two or more BTNs to form a complex, for example, refolding a BTN2 comprising a C-terminal dimerization peptide and BTN3 comprising a C-terminal dimerization peptide to provide a BTN2/BTN3 heterodimer complex.

Any purification method known in the art can be used, for example, by chromatography (e.g., ion exchange chromatography (e.g., hydroxylapatite chromatography), affinity chromatography, particularly Protein A, Protein G or lectin affinity chromatography, sizing column chromatography), centrifugation, differential solubility, hydrophobic interaction chromatography, or by any other standard technique for the purification of proteins. The skilled person will readily be able to select a suitable purification method based on the individual characteristics of the BTN or variant or fragment thereof, or BTN construct, or BTN complex, or single chain fusion polypeptide to be recovered.

### Effector Host Cell

The present disclosure also provides for “effector host cells” comprising a nucleotide sequence, vector or BTN or BTN complex of the disclosure. Said effector host cells are modified using routine methods to comprise a nucleic acid sequence encoding a BTN or variant or fragment thereof, or BTN construct, or BTN complex, or single chain fusion polypeptide of the disclosure, and are envisaged to express the BTN or BTN complex described herein, in particular on the cell surface. For the purposes of the present disclosure, “modified host cells expressing a BTN or BTN complex of the disclosure” generally refers to (target or production) host cells treated or altered to express a BTN or BTN complex according to the present disclosure, for instance by RNA transfection of nucleic acid(s) or vector(s) or the disclosure. Other methods of modification or transfection or transduction, such as those described elsewhere herein, are also envisaged. The term “modified host cell” thus includes “transfected”, “transduced” and “genetically engineered” host cells preferably expressing the BTN or BTN complex of the present disclosure.

Preferably, such host cells (in particular “modified antigen presenting cells”) are capable of mediating T cell effector functions upon binding of the BTN to the T cell. Such T cell effector functions include for instance the release of perforin (which creates holes in the target cell membrane), granzymes (which are proteases that act intracellularly to trigger apoptosis), the expression of Fas ligand (which activates apoptosis in a Fas-bearing target cell) and the release of cytokines, preferably Th1/Tc1 cytokines such as IFN-γ, IL-2 and TNF-α. Cytolysis of target cells can be assessed, for example, with the CTL fluorescent killing assay detecting the disappearance of fluorescently labelled target cells during co-culture with T cells.

Effector host cells in particular antigen presenting cells such as dendritic cells, macrophages, Langerhans cells and B cells can be autologous host cells that are obtained from the subject to be treated and transformed or transduced to express the BTN or BTN complex of the disclosure. Typically, recombinant expression of the BTN or BTN complex will be accomplished by using a viral vector. Techniques for obtaining and isolating the cells from the patient are known in the art.

The effector host cells provided herein are particularly envisaged for therapeutic applications. Further genetic modifications of the host cells may be desirable in order to increase therapeutic efficacy, for example, when using autologous dendritic cells, macrophages, Langerhans cells and B cells as “effector host cells” suitable additional modifications include downregulation of the endogenous BTN expression; and/or amplification of co-stimulatory molecules such as other BTN molecules, ApoAI, RhoB, ABCA1, and/or periplakin. Means and methods for achieving the aforementioned genetic modifications have been described in the art. Methods for targeted genome engineering of host cells are known in the art and include, besides gene knockdown with siRNA, the use of so-called “programmable nucleases” such as zinc-finger nucleases (ZFNs), transcription activator-like effector nucleases (TALENs) and RNA-guided engineered nucleases (RGENs) derived from the bacterial clustered regularly interspaced short palindromic repeat (CRISPR)-Cas (CRISPR-associated) system. For instance, programmable nucleases such as TALENs can be employed to cut the DNA regions that code for “unwanted” proteins, such as endogenous BTN2 or BTN3, and thereby reduce their expression. When dendritic cells, macrophages, Langerhans cells and B cells are used as (effector) host cells, downregulation of the endogenous BTN has the benefit of reducing unwanted “mispairing” of endogenous and exogenous BTN chains.

### Soluble BTN and BTN2/BTN3 Complexes

In one example, the modified BTN or BTN complex of the disclosure is a soluble BTN or BTN complex.

A soluble BTN complex useful in the disclosure typically is a heterodimer comprising a BTN2 chain (e.g., BTN2A1) and a BTN3 chain (e.g., BTN3A1 chain) but multimers (e.g., tetramers) comprising two different heterodimers or two of the same heterodimers are also contemplated for use in the present disclosure.

A soluble BTN or BTN complex of the disclosure may be provided in substantially pure form, or as a purified or isolated preparation. For example, it may be provided in a form which is substantially free of other proteins.

A plurality of soluble BTNs or BTN complexes of the present disclosure may be provided in a multivalent complex. Thus, the present disclosure provides, in one aspect, a multivalent BTN complex, which comprises a plurality of soluble BTNs as described herein.

In its simplest form, a multivalent BTN complex according to the invention comprises a multimer of two or three or four or more BTNs associated (e.g. covalently or otherwise linked) with one another, preferably via a linker molecule. Suitable linker molecules include, but are not limited to, multivalent attachment molecules such as avidin, streptavidin, neutravidin and extravidin, each of which has four binding sites for biotin. Thus, biotinylated BTN molecules can be formed into multimers of BTNs having a plurality of BTN binding sites. The number of BTN molecules in the multimer will depend upon the quantity of BTN in relation to the quantity of linker molecule used to make the multimers, and also on the presence or absence of any other biotinylated molecules. Preferred multimers are dimeric, trimeric or tetrameric BTN complexes.

The BTNs may be complexed to a structure for use. Suitable structures for forming complexes with one or a plurality of TCRs include membrane structures such as liposomes and solid structures which are preferably particles such as beads, for example latex beads. Other structures which may be externally coated with BTNs are also suitable. Preferably, the structures are coated with BTN multimers rather than with individual BTNs.

In the case of liposomes, the BTNs or multimers thereof may be attached to or otherwise associated with the membrane. Techniques for this are well known to those skilled in the art.

A label or another moiety, such as a toxic or therapeutic moiety, may be included in a multivalent BTN complex of the disclosure. For example, the label or other moiety may be included in a mixed molecule multimer. Such mixed molecules may contain any combination of molecules, provided that steric hindrance does not compromise or does not significantly compromise the desired function of the molecules. The positioning of the binding sites on the streptavidin molecule is suitable for mixed tetramers since steric hindrance is not likely to occur.

In particular, the soluble BTN or multivalent BTN complex can be used to activate cells that express a δ2⁺ TCR (e.g., γδ T cells). This would be useful in many situations and, in particular, to activate γδ T cells ex vivo prior to administration to a patient, for example, to prevent, treat, delay the progression of, prevent a relapse of, or alleviate a symptom of a cancer or an infection

Soluble BTNs and BTN complexes of the present disclosure can be produced by any suitable method known to those of skill in the art and are most typically produced recombinantly. According to the present disclosure, a recombinant nucleic acid molecule useful for producing a soluble BTN or BTN single chain fusion protein typically comprises a recombinant vector and a nucleic acid sequence encoding one or more BTNs.

According to the present disclosure, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences which encode a protein of interest or which are useful for expression of the nucleic acid molecules. The vector can be either RNA or DNA, either prokaryotic or eukaryotic, and typically is a plasmid.

Typically, a recombinant nucleic acid molecule includes at least one nucleic acid molecule of the present invention operatively linked to one or more transcription control sequences. As used herein, the phrase “recombinant molecule” or “recombinant nucleic acid molecule” primarily refers to a nucleic acid molecule or nucleic acid sequence operatively linked to a transcription control sequence but can be used interchangeably with the phrase “nucleic acid molecule”, when such nucleic acid molecule is a recombinant molecule as discussed herein. According to the present disclosure, the phrase “operatively linked” refers to linking a nucleic acid molecule to a transcription control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced.

One or more recombinant molecules of the present invention can be used to produce an encoded product (e.g., a soluble BTN2/BTN3 complex) of the present disclosure. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., yeast), insect, plant or animal cells that can be transfected. Host cells can be either untransfected cells or cells that are already transfected with at least one other recombinant nucleic acid molecule. Resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present disclosure can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins produced according to the present disclosure are preferably retrieved in “substantially pure” form. As used herein, “substantially pure” refers to a purity that allows for the effective use of the soluble BTN or BTN complex in a composition and method of the present disclosure.

By way of example, recombinant constructs containing the relevant BTN2 and BTN3 genes (e.g., nucleic acid sequences encoding the desired portions of the BTN2 and BTN3 chains of a BTN2/BTN3 complex) can be synthesized de novo or can be produced by PCR of BTN cDNAs derived from a source of cells such as antigen presenting cells that express the desired BTN. The PCR amplification of the desired BTN2 and BTN3 genes can be designed so that the transmembrane and cytoplasmic domains of the chains will be omitted (i.e., creating a soluble complex). In addition, if desired, sequence encoding a selectable marker for purification or labeling of the product or the constructs can be added to the constructs. Amplified BTN2 and BTN3 cDNA pairs are then cloned, sequence-verified, and transferred into a suitable vector.

The soluble BTN2/BTN3 DNA constructs are then co-transfected into a suitable host cell (e.g., in the case of a baculoviral vector, into suitable insect host cells or in the case of a mammalian expression vector, into suitable mammalian host cells) which will express and secrete the recombinant receptors into the supernatant, for example. Culture supernatants containing soluble BTN2/BTN3 can then be purified using various affinity columns, such as nickel nitrilotriacetic acid affinity columns. The products can be concentrated and stored. It will be clear to those of skill in the art that other methods and protocols can be used to produce soluble BTNs or BTN complexes for use in the present disclosure, and such methods are expressly contemplated for use herein.

In one example, the BTN complex of the disclosure is a single chain fusion polypeptide.

A single chain fusion polypeptide useful in the disclosure typically is a heterodimer comprising a BTN2 chain (e.g., BTN2A1) fused to a BTN3 chain (e.g., BTN3A1 chain). In some embodiments, the BTN2 chain and BTN3 chain are separated by a peptide linker.

Linkers useful in the fusion polypeptides of the disclosure serve to connect the BTN chains. The linkers may also maintain cooperative inter-domain interaction or preserve biological activity.

The linker may be about 25-275 amino acid residues in length. The linker may be about 50-250 amino acids in length, about 75-225 amino acids in length, about 100-200 amino acids in length, about 125-175 amino acids in length, about 25-100 amino acids in length, about 50-150 amino acids in length, about 150-250 amino acids in length about 5-25 amino acids in length. In some embodiments, the linker is a polypeptide having a sequence of about 5 amino acid in length, about 10 amino acids in length, about 15 amino acids in length, about 20 amino acids in length, or about 25 amino acids in length.

In some embodiments, the linker comprises (for example, at least about 50% of the amino acids are) polar, uncharged or charged residues, for example, one or more of threonine (Thr), serine (Ser), proline (Pro), glycine (Gly), aspartic acid (Asp), lysine (Lys), glutamine (Gln), asparagine (Asn), and alanine (Ala); or one or more of Pro, arginine (Arg), phenylalanine (Phe), threonine (Thr), glutamic acid (Glu) and glutamine (Gln).

In some embodiments, the linker comprise proline (Pro), threonine (Thr), and/or glutamine (Gln) residues, for example, about 50% of the amino acids comprise proline (Pro), threonine (Thr), and/or glutamine (Gln) residues. Among them, Pro is a unique amino acid with a cyclic side chain which causes a very restricted conformation. The lack of amide hydrogen on Pro may prevent the formation of hydrogen bonds with other amino acids, and therefore reduces the interaction between the linkers and the protein domains. As a result, the inclusion of Pro residues might increase the stiffness and structural independence of the linkers. The small, polar amino acids, such as Thr, or Ser and Gly might provide good flexibility due to their small sizes, and also help maintain stability of the linker structure in the aqueous solvent through formation of hydrogen bonds with water.

In some embodiments, the linker adopts a helical, β-strand, or coil/bend secondary structure. The linker may be helical or non-helical. The linker may form an α-helix. Some α-helical conformations form rapidly during folding, allowing the correct folding of connecting protein domains without non-native interactions with the linker. Linkers in an α-helix structure might also serve as rigid spacers to effectively separate protein domains, and to reduce their unfavorable interactions. On the other hand, without an inherent rigid structure, the non-helical linkers may be rich in Pro, which could increase the stiffness of the linker. As such, non-helical linkers with Pro-rich sequence could exhibit relatively rigid structures and serve to reduce inter-domain interference.

In some embodiments, a flexible linker is used. Flexible linkers are usually applied when the joined domains require a certain degree of movement or interaction. They are generally composed of small, non-polar (e.g. Gly) or polar (e.g. Ser or Thr) amino acids. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties.

The most commonly used flexible linkers have sequences consisting primarily of stretches of Gly and Ser residues (“GS” linker). An example of the most widely used flexible linker has the sequence of (Gly-Gly-Gly-Gly-Ser)n. By adjusting the copy number “n”, the length of this GS linker can be optimized to achieve appropriate separation of the functional domains, or to maintain necessary inter-domain interactions. Besides the GS linkers, many other flexible linkers have been designed for recombinant fusion proteins. These flexible linkers are also rich in small or polar amino acids such as Gly and Ser, but can contain additional amino acids such as Thr and Ala to maintain flexibility, as well as polar amino acids such as Lys and Glu to improve solubility.

In some embodiments a rigid linker may be used for example, to provide a fixed distance between the domains and to maintain their independent functions. For example, alpha helix-forming linkers with the sequence of (EAAAK)n have been applied to the construction of many recombinant fusion proteins. Another type of rigid linkers useful in the single chain fusion polypeptides of the disclosure has a Pro-rich sequence, (XP)n, with X designating any amino acid, preferably Ala, Lys, or Glu.

In some embodiments, the linker is a stable peptide sequence. These stable linkers covalently join functional domains together to act as one molecule.

In other embodiments, the linker may be cleavable. Such linkers may be cleaved under specific conditions such as the presence of reducing reagents or proteases. This type of linker may reduce steric hindrance, improve bioactivity, or achieve independent actions/metabolism of individual domains of recombinant fusion proteins after linker cleavage.

Suitable linkers can be designed by computation methods and computational graphics.

The single chain fusion polypeptide may additionally comprise an N-terminal signal peptide domain, which allows processing, e.g., extracellular secretion, in a suitable host cell. Preferably, the N-terminal signal peptide domain comprises a protease cleavage site, for example, a signal peptidase cleavage site and thus may be removed after or during expression to obtain the mature protein. Further, the fusion protein may additionally comprise a C-terminal element, having a length of e.g. 1-50, preferably 10-30 amino acids which may include or connect to a recognition/purification domain, e.g., a FLAG domain, a Strep-tag or Strep-tag II domain and/or a poly-His domain.

Further, the fusion polypeptide may additionally comprise N-terminally and/or C-terminally a further domain, for example, a targeting domain such as a single-chain antibody or an antibody fragment domain. Suitable examples of other targeting molecules are cytokines, such as interleukins.

### Functional Measures of γδ T Cell Immune Responses

The present disclosure relates to a modified BTN or BTN complex that can activate T cells expressing a δ2⁺ TCR (e.g., a Vγ9Vδ2⁺ TCR) and enhance cytolytic function, cytokine production of one or more cytokines and/or proliferation of said T cells. In some embodiments the T cells are γδ T cells, for example, Vδ1+ or Vδ2+ T cells. In other embodiments, the T cells are αβ T cells, for example, CD4+ or CD8+ T cells. T-cell number and function may be monitored by assays that detect T cells by an activity such as cytokine production, proliferation, or cytotoxicity. Such activity may be correlated with clinical outcome. For example, activation of cytolytic activity may result in lysis of tumor targets or infected cells. Activation and increased cytokine production may lead to cytokine-induced cell death of tumor or other targets.

By enhancing the cytolytic function of T cells, it is meant an increase of the cytotoxicity of T cells, i.e., an increase of the specific lysis of the target cells by T cells. The cytolytic function of T cells can be measured by, for example, direct cytotoxicity assays. A cytotoxicity assay typically involves mixing a sample containing effector cells with targets (e.g., K562 cells) loaded with ⁵¹Cr or europium and measuring the release of the chromium or europium after target cell lysis. Surrogate targets are often used, such as tumor cell lines. The targets can be loaded with an antigen, for example, a pAg. The percentage of lysis of the targets after incubation for approximately 4 hours is calculated by comparison with the maximum achievable lysis of the target. Cytotoxicity assays can be used for monitoring the activity of passively delivered effector cells and active immunotherapy approaches.

By activating cytokine production of one or more cytokines by T cells, it is meant an increase in total cytokine production of one or more particular cytokines (for example, IFN-γ, TNF-α, GM-CSF, IL-2, IL-6, IL-8, IP-10, MCP-1, MIP-1α, MIP-1β or IL-17A) by γδ T cells. Cytokine secretion by T cells may be detected by measuring either bulk cytokine production (by an ELISA), by bead based assays (e.g., Luminex), or enumerating individual cytokine producing T cells (by an ELISPOT assay).

In an ELISA assay, effector cells are incubated with or without target cells and after a defined period of time, the supernatant from the culture is harvested and added to microtiter plates coated with antibody for cytokines of interest. Antibodies linked to a detectable label or reporter molecule are added, and the plates washed and read. Typically, a single cytokine is measured in each well, although up to 15 cytokines can be measured in a single sample. Antibodies to cytokines of interest may be covalently bound to microspheres with uniform, distinctive proportions of fluorescent dyes. Detection antibodies conjugated to a fluorescent reporter dye are then added, and flow cytometry performed. By gating on a particular fluorescence indicating a particular cytokine of interest, it is possible to quantify the amount of cytokine that is proportional to the amount of reporter fluorescence.

In a bead based assay like Luminex, the sample is usually added to a mixture of color-coded beads, pre-coated with analyte-specific capture antibodies. The antibodies bind to the analytes of interest. Biotinylated detection antibodies specific to the analytes of interest are added and form an antibody-antigen sandwich. Fluorophore-conjugated streptavidin is added and binds to the biotinylated detection antibodies. Beads are read on a flow-based detection instrument. One laser classifies the bead and determines the analyte that is being detected. The second laser determines the magnitude of the fluorophore-derived signal, which is in direct proportion to the amount of analyte bound.

An ELISPOT assay typically involves coating a 96-well microtiter plate with purified cytokine-specific antibody; blocking the plate to prevent nonspecific absorption of random proteins; incubating the cytokine-secreting T cells with stimulator cells at several different dilutions; lysing the cells with detergent; adding a labeled second antibody; and detecting the antibody-cytokine complex. The product of the final step is usually an enzyme/substrate reaction producing a colored product that can be quantitated microscopically, visually, or electronically. Each spot represents one single cell secreting the cytokine of interest.

Cytokine production of one or more cytokines by γδ T cells can also be detected by multiparameter flow cytometry. Here, cytokine secretion is blocked for 4-24 hours with Brefeldin A or Monensin (both protein transport inhibitors that act on the Golgi in different ways, which one is best depends on the cytokine to examine) in γδ T cells before the cells are surface stained for markers of interest and then fixed and permeabilized followed by intracellular staining with fluorophore-coupled antibodies targeting the cytokines of interest. Afterwards the cells can be analyzed by Flow-cytometry. It is possible to monitor immune responses in humans by characterizing the cytokine secretion pattern of T cells in peripheral blood, lymph nodes, or tissues by flow cytometry. This can be done ex-vivo without BFA or Monensin treatment.

By activating proliferation of γδ T cells, it is meant an increase in number of γδ T cells. Proliferation can be measured using a lymphoproliferative assay. A sample of effector cells is mixed with various dilutions of stimulator cells. After 72-120 h, [³H]thymidine is added, and DNA synthesis (as a measure of proliferation) can be quantified by using a gamma counter to measure the amount of radiolabeled thymidine incorporated into the DNA.

### Indications

The present disclosure relates to modified BTNs or BTN complexes which can be used to prevent, treat, delay the progression of, prevent a relapse of, or alleviate a symptom of a disease or condition.

A method for the treatment of disease relates to the therapeutic use of a modified BTN or BTN complex, vector or effector cell of the disclosure. In this respect, the modified BTN or BTN complex, vector encoding the BTN or BTN complex or effector cell comprising the BTN or BTN complex may be administered to a subject to prevent, treat, delay the progression of, prevent a relapse of, or alleviate a symptom of a disease or condition.

In some embodiments, the methods include isolating cells from a donor (allogeneic) or patient (autologous), preparing, processing, culturing, and/or engineering them, as described herein (to provide effector cells), and introducing or re-introducing them into the patient, before or after cryopreservation.

Alternatively, effector cells may be derived from ex-vivo differentiation of inducible progenitor cells or embryonic progenitor cells to lineage specific cells.

The modified BTN or BTN complexes, vectors or effector cells of the disclosure can be used to prevent, treat, delay the progression of, prevent a relapse of, or alleviate a symptom of cancer.

The modified BTN or BTN complexes, vectors or effector cells can also be used to prevent, treat, delay the progression of, prevent a relapse of, or alleviate a symptom of infection.

The BTN or BTN complexes, vectors or effector cells can be used to prevent, treat, delay the progression of, prevent a relapse of, or alleviate a symptom of autoimmune disease. In one embodiment, the modified BTN or BTN complexes are used to deliver a cytotoxic agent to γδ T cells.

The BTN or BTN complexes, vectors or effector cells of the disclosure may optionally be used may be used in combination with other immunosuppressive and chemotherapeutic agents such as, but not limited to, prednisone, azathioprine, cyclosporin, methotrexate, and cyclophosphamide.

The BTN or BTN complexes, vectors or effector cells can be administered intravenously, intramuscularly, subcutaneously, transdermally, intraperitoneally, intrathecally, parenterally, intrathecally, intracavitary, intraventricularly, intra-arterially, or via the cerebrospinal fluid, or by any implantable or semi-implantable, permanent or degradable device. The appropriate dosage may be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate.

### Pharmaceutical Compositions

Suitably, in compositions or methods for administration of the BTN or BTN complexes, vectors or effector cells to a subject, the BTN or BTN complexes, vectors or effector cells are combined with a pharmaceutically acceptable carrier as is understood in the art. Accordingly, one example of the present disclosure provides a composition (e.g., a pharmaceutical composition) comprising the BTN or BTN complexes, vectors or effector cells combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

In one example, the BTN or BTN complexes, vectors or effector cells are administered parenterally, such as subcutaneously or intravenously. For example, the BTN or BTN complexes, vectors or effector cells are administered intravenously. In some examples, the BTN or BTN complexes, vectors or effector cells are administered intra-tumorally.

Formulation of a BTN or BTN complex, vector or effector cell to be administered will vary according to the route of administration and formulation (e.g., solution, emulsion, capsule) selected. An appropriate pharmaceutical composition comprising a BTN or BTN complex, vector or effector cell to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate.

The BTN or BTN complex, vector or effector cell can be stored in the liquid stage or can be lyophilized for storage and reconstituted in a suitable carrier prior to use according to art-known lyophilization and reconstitution techniques.

### EXAMPLES

To understand the molecular mode of BTN engagement by γδTCR, the present inventors solved the crystal structures of BTN2A1 ectodomain either alone (‘apo’), or in complex with Vγ9Vδ2⁺γδTCR, which diffracted to 3.6 Å and 2.1 Å resolution, respectively (Table 1).

TABLE 1
Data collection and refinement statistics.

| | γδTCR-BTN2A1 | | Apo BTN2A1 | | BTN2A1-BTN3A1 | |
|---|---|---|---|---|---|---|
| Data collection | | | | | | |
| Radiation source | Australian | | Australian | | Australian | |
| | Synch. MX2 | | Synch. MX2 | | Synch. MX2 | |
| Wavelength (Å) | 0.95372 | | 0.95372 | | 0.95372 | |
| Resolution range (Å) | 49.7-2.10 | | 49.8-3.55 | | 47.4-5.55 | |
| | (2.14-2.10) | | (3.89-3.55) | | (6.62-5.55) | |
| Ellipsoid resolution (Å) | — | | 4.30 | | 5.93 (a*) | |
| (direction)^{a} | | | (0.95 a* + | | | |
| | | | 0.33 c*) | | | |
| | — | | 3.92 | (b*) | 5.28 | (b*) |
| | — | | 3.55 | | 8.24 | (c*) |
| | | | (−0.80 a* + | | | |
| | | | 0.60 c*) | | | |
| Best/Worst diffraction limits(Å)^{a} | — | | 3.55 / 5.62 | | 5.55 / 8.85 | |
| Space group | C 2 2 2₁ | | C 2 | | F 2 2 2 | |
| Unit cell parameters: a, b, c (Å) | 112.0, 218.5, 107.9 | | 237.0, 94.2, 134.7 | | 114.6 138.9 336.2 | |
| α, β, γ (°) | 90, 90, 90 | | 90, 106.35, 90 | | 90, 90, 90 | |
| Data processing | | | | | | |
| Total observations (No.) | 1,038,721 | (53,094) | 101,471 | (5,622)^{a} | 18,589 | (2,124)^{a} |
| Unique observations (No.) | 77,369 | (3,834) | 22,300 | (1,115)^{a} | 2,500 | (278)^{a} |
| Multiplicity | 13.4 | (13.8) | 4.6 | (5.0)^{a} | 7.4 | (7.6)^{a} |
| Data completeness (isotropic, %) | 99.97 | (100.00) | 64.3 | (13.5)^{a} | 56.5 | (15.6)^{a} |
| Data completeness (ellipsoidal, %)^{a} | — | | 85.2 | (70.0)^{a} | 85.9 | (49.2)^{a} |
| Mean I/σ_{I} | 13.78 | (2.84) | 5.8 | (2.0)^{a} | 5.3 | (1.6)^{a} |
| Wilson B-factor (Å²) | 36 | | 111 | | 186 | |
| R_{merge} (%) | 12.0 | (108) | 15.3 | (78.6)^{a} | 20.2 | (145.6)^{a} |
| Rₘₑₐₛ (%) | 12.4 | (112.1) | 17.3 | (87.5)^{a} | 22.1 | (156.2)^{a} |
| Rₚᵢₘ (%) | 3.37 | (29.9) | 10.8 | (51.3)^{a} | 8.6 | (55.8)^{a} |
| CC_{1/2} (%) | 99.8 | (72.5) | 98.0 | (64.0)^{a} | 91.9 | (37.3)^{a} |

| Refinement statistics | | | | |
|---|---|---|---|---|
| Resolution range (Å)^{b} | 49.7-2.10 | | 49.8-3.55 | 47.4-5.55 |
| | (2.14-2.10) | | (3.76-3.55) | (6.88-5.55) |
| Reflections used in refinement^{b} | 77,362 | (3,834) | 22,300 | 2,500 |
| Reflections used for R-free^{b} | 3,723 | (189) | 1,144 | 138 |
| R_{work} (%)^{b} | 26.1 | (33.6) | 25.5 | 29.1 |
| R_{free} (%)^{b} | 28.7 | (35.9) | 26.8 | 32.8 |

| | | | |
|---|---|---|---|
| Number of non-hydrogen atoms | 7,186 | 8993 | 3305 |
| macromolecules | 6,848 | 8524 | 3277 |
| ligands | 173 | 469 | 28 |
| solvent | 165 | 0 | 0 |
| Protein residues | 868 | 1087 | 425 |
| RMSD (bond length)^{b} | 0.008 | 0.008 | 0.007 |
| RMSD (bond angle)^{b} | 1.02 | 0.99 | 0.91 |
| Ramachandran - favored (%) | 96.4 | 96.75 | 95.23 |
| allowed (%) | 3.49 | 3.25 | 4.77 |
| outliers (%) | 0.12 | 0 | 0 |
| Rotamer outliers (%) | 3.66 | 4.38 | 3.37 |
| Clashscore | 5.15 | 6.89 | 9.59 |
| Average B-factor | 58.2 | 268.94 | 283.85 |
| macromolecules | 58.29 | 268.29 | 283.93 |
| ligands | 70.48 | 280.87 | 274.89 |
| solvent | 41.66 | — | — |

^{a}Calculated by the Staraniso server (Global Phasing). The cut-off surface is unlikely to be perfectly ellipsoidal, so this is only an estimate.
^{b}Calculated by Buster (Global Phasing).

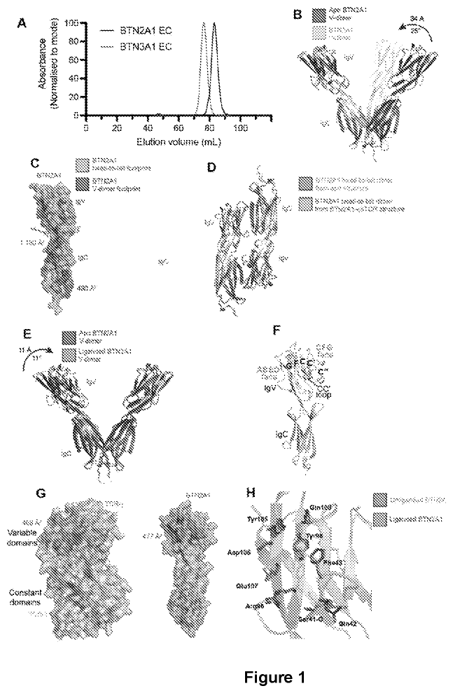

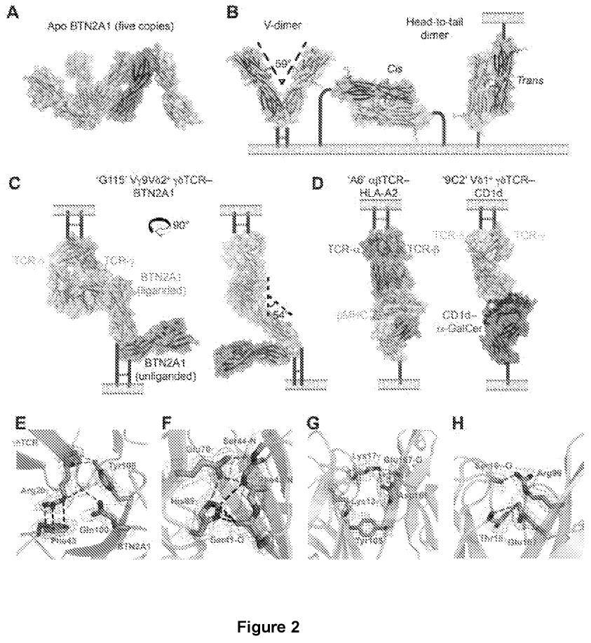

BTN2A1 is reported to exist on the cell surface predominantly as a homodimer, which is stabilized by a membrane-proximal interchain disulfide bond (M. M. Karunakaran et al., Butyrophilin-2A1 Directly Binds Germline-Encoded Regions of the Vgamma9Vdelta2 TCR and Is Essential for Phosphoantigen Sensing. Immunity 52, 487-498 e486 (2020)). The exemplified BTN2A1 construct lacked the terminal Cys residue responsible for this disulfide bond and consequently appeared to exist in solution as a free monomer (FIG. 1A). Nonetheless, the apo form of BTN2A1 contained five copies in the asymmetric unit, arranged as two head-to-tail V-shaped homodimers (‘V-dimers’) (FIG. 2A), with the fifth copy also forming a V-dimer via crystallographic symmetry. This V-dimer was broadly reminiscent of the BTN3A1 V-dimer (A. Palakodeti et al., The molecular basis for modulation of human Vgamma9Vdelta2 T cell responses by CD277/butyrophilin-3 (BTN3A)-specific antibodies. 287, 32780-32790 (2012), although the BTN2A1 V-dimers formed at an angle of 59°, which is significantly wider than BTN3A1 V-dimers (49°), and the BTN2A1 V-dimers were also twisted by 25° compared to BTN3A1 V-dimers (A. Palakodeti et al., The molecular basis for modulation of human Vgamma9Vdelta2 T cell responses by CD277/butyrophilin-3 (BTN3A)-specific antibodies. 287, 32780-32790 (2012)). (FIG. 2B and FIG. 1B). The V-dimer was characterized by a small interface dominated by a limited array of primarily non-polar interactions including three π-mediated interactions, with a buried surface area (BSA) of ˜430 Å² per molecule (FIG. 1C and Table 2).

TABLE 2
BTN2A1 V-dimer contacts.

| BTN2A1 copy | BTN2A1 copy | BTN2A1 copy | BTN2A1 copy | |
|---|---|---|---|---|
| #1 region | #1 residues | #2 region | #2 residues | Bond type |
| IgC A-strand | Met125 | Juxta-membrane | Pro209, Phe212 | VDW |
| IgC A-strand | Arg126 | IgC G-strand | Phe207 | Cation-π |
| IgC A-strand | Arg126 | IgC G-strand, | Phe207, Pro209 | VDW |
| | | juxta-membrane | | |
| IgC A-strand | Gly127 | Juxta-membrane | Pro209, Phe212 | VDW |
| IgC A-strand | His128-Nδ1 | Juxta-membrane | Ser211 Oγ | HB |
| IgC A-strand | His128-N | Juxta-membrane | Ser211-Oγ | Water- |
| | | | | mediated HB |
| IgC A-strand | His128 | Juxta-membrane | Ser211, Phe212 | VDW |
| IgC G-strand | Phe207 | IgC A-strand | Arg126 | Cation-π |
| IgC G-strand | Phe207 | IgC A-strand | Arg126 | VDW |
| Juxta-membrane | Pro209 | IgC A-strand | Met125, Arg126, | |
| | | | Gly127 | |
| Juxta-membrane | Ser211-Oγ | IgC A-strand | His128-Nδ1 | HB |
| Juxta-membrane | Ser211-Oγ | IgC A-strand | His128-N | Water- |
| | | | | mediated HB |
| Juxta-membrane | Ser211 | IgC A-strand | His128 | VDW |
| Juxta-membrane | Phe212 | Juxta-membrane | Phe212 | π-π stacking |
| Juxta-membrane | Phe212 | IgC A-strand | Met125, Gly127, | VDW |
| | | | His128 | |

Van der Waals (VDW) defined as non-hydrogen bond contact distances of 4 Å or less, hydrogen bonds (HB) 3.5 Å or less, cation-π and π-π as 4.5 Å or less and water-mediated HB 3.3 Å or less.

A head-to-tail dimer of BTN2A1 was also observed in both the apo structure (FIG. 2B) and BTN2A1-γδTCR complex (FIG. 1D), although the latter only involved the unliganded BTN2A1 copy, via crystallographic symmetry, because the head-to-tail footprint overlapped with the γδTCR binding site. The head-to-tail dimer had a larger BSA of ˜1180 Å² per molecule compared to the V-dimer (FIG. 1C and Table 3), and could potentially form following either a cis or a trans interaction (FIG. 2B), akin to the purported BTN3A1 head-to-tail homodimer (A. Palakodeti et al., The molecular basis for modulation of human Vgamma9Vdelta2 T cell responses by CD277/butyrophilin-3 (BTN3A)-specific antibodies. 287, 32780-32790 (2012)).

TABLE 3
BTN2A1 head-to-tail dimer contacts.

| BTN2A1 copy | BTN2A1 copy | BTN2A1 copy | BTN2A1 copy | |
|---|---|---|---|---|
| #1 region | #1 residues | #2 region | #2 residues | Bond type |
| N-terminus | Gln1-Nε2 | IgC D-strand | Lys162-Nζ | HB |
| N-terminus | Gln1 | IgC A/D-strands | Glu129, Lys162 | VDW |
| IgV C-strand | Phe39 | IgC D-strand | Met166, Pro167 | VDW |
| IgV CC′-loop | Ser41 | IgC D/E-strands | Pro167, Gly171 | VDW |
| IgV CC′-loop | Gln42-Nε2 | IgC DE-loop | Ala169-O | HB |
| IgV CC′-loop | Gln42 | IgV EF-loop, | Gln90, Asp168, | VDW |
| | | IgC D-strand, | Ala169, Gly171 | |
| | | IgC DE-loop, | | |
| | | IgC E-strand | | |
| IgV CC′-loop | Phe43-N | IgC D-strand | Asp168-O | HB |
| IgV CC′-loop | Phe43 | IgC D-strand, | Met166, Pro167, | VDW |
| | | IgC DE-loop | Asp168, Ala169 | |
| IgV CC′-loop | Ser44 | IgC DE-loop | Ala169 | VDW |
| IgV EF-loop | Gln90 | IgV CC′-loop | Gln42 | VDW |
| IgV glycans | (Asn92) | IgV glycans | (Asn92) | HB |
| | NAG221-O6 | | NAG221-O6 | |
| IgV glycans | (Asn92) NAG221 | IgV glycans | (Asn92) NAG221 | VDW |
| IgV F-strand | Arg96 | IgC D/E-strands | Pro167, Phe173 | VDW |
| IgV F-strand | Tyr98 | IgC D-strand | Met166 | VDW |
| IgV FG-loop | Arg103-Nε | IgC B-strand | Glu136-Oε1, | SB |
| | | | Glu136-Oε2 | |
| IgV FG-loop | Arg103-Nη1 | IgC B-strand | Glu136-Oε1 | SB |
| IgV FG-loop | Arg103 | IgC A/B-strands | Arg126, Glu136 | VDW |
| IgV G-strand | Tyr105 | IgC D/E-strands | Val164, Met174, | VDW |
| | | | Thr176 | |
| IgV G-strand | Asp106 | IgC D-strand | Val164 | VDW |
| IgV G-strand | Glu107 | IgC D-strand | Ser165 | VDW |
| IgC A-strand | Arg126 | IgV FG-loop | Arg103 | VDW |
| IgC A-strand | Glu129 | N-terminus | Gln1 | VDW |
| IgC B-strand | Glu136-Oε1 | IgV FG-loop | Arg103-Nε, | SB |
| | | | Arg103-Nη1 | |
| IgC B-strand | Glu136-Oε2 | IgV FG-loop | Arg103-Nε | SB |
| IgC B-strand | Glu136 | IgV FG-loop | Arg103 | VDW |
| IgC D-strand | Lys162 | N-terminus | Gln1 | VDW |
| IgC D-strand | Val164 | IgV G-strand | Tyr105, Asp106 | VDW |
| IgC D-strand | Ser165-N | IgV G-strand | Glu107-Oε1 | Water- |
| | | | | mediated HB |
| IgC D-strand | Ser165 | IgV G-strand | Glu107 | VDW |
| IgC D-strand | Met166 | IgV C-strand, | Phe39, Phe43, | VDW |
| | | CC′-loop, F-strand | Tyr98 | |
| IgC D-strand | Pro167 | IgV C-strand, | Phe39, Ser41, | VDW |
| | | CC′-loop, F-strand | Phe43, Arg96 | |
| IgC D-strand | Asp168-O | IgV CC′-loop | Phe43-N | HB |
| IgC D-strand | Asp168 | IgV CC′-loop | Gln42, Phe43 | VDW |
| IgC DE-loop | Ala169-O | IgV CC′-loop | Gln42-Nε2 | HB |
| IgC DE-loop | Ala169 | IgV CC′-loop | Gln42, Phe43, | VDW |
| | | | Ser44 | |
| IgC E-strand | Gly171 | IgV CC′-loop | Ser41, Gln42 | VDW |
| IgC E-strand | Phe173 | IgV F-strand | Arg96 | VDW |
| IgC E-strand | Met174 | IgV G-strand | Tyr105 | VDW |
| IgC E-strand | Thr176 | IgV G-strand | Tyr105 | VDW |

Van der Waals (VDW) defined as non-hydrogen bond contact distances of 4 Å or less, hydrogen bonds (HB) as 3.5 Å or less, salt bridge (SB) as 4.5 Å or less and water-mediated HB 3.3 Å or less.

The asymmetric unit of the complex contained two copies of BTN2A1, also arranged as a V-dimer that was similar to the apo BTN2A1 V-dimers (FIG. 1E) with one of the BTN2A1 copies liganded to the γδTCR, and the other one remaining unliganded (FIG. 2C). BTN2A1 engaged the side of the γ-chain, binding to the Vγ9-encoded IgV domain, jutting out at an angle of ˜54°, which starkly contrasted αβTCR engagement of pMHC, or γδTCR recognition of CD1d (FIG. 2D). Previous studies implicated the hypervariable region 4 (HV4) loop, also known as the DE loop of Vγ9, as well as the CDR36 loop, in binding BTN molecules (M. M. Karunakaran et al., Butyrophilin-2A1 Directly Binds Germline-Encoded Regions of the Vgamma9Vdelta2 TCR and Is Essential for Phosphoantigen Sensing. Immunity 52, 487-498 e486 (2020); C. R. Willcox et al., Butyrophilin-like 3 Directly Binds a Human Vgamma4(+) T Cell Receptor Using a Modality Distinct from Clonally-Restricted Antigen. Immunity 51, 813-825 e814 (2019); A. Vγδ orova et al., gamma9delta2T cell diversity and the receptor interface with tumor cells. 130, 4637-4651 (2020)).

The crystal structure revealed that the BTN2A1 binding site on Vγ9 was distal to both the CDR and HV4 loops (>7 Å and >9 Å separation, respectively), and instead left the entire apical surface of the γδTCR solvent exposed (FIG. 2C). The outer face of the Vγ9 germline-encoded β-sheet formed by the A, B, D and E β-strands (ABED face) mediated binding to the β-sheet encoded by the C, F and G β-strands (CFG face) of the BTN2A1 IgV domain (FIG. 1F), with energetic contributions by all these strands (FIG. 1G). The γδTCR buried 468 Å² upon ligation, and BTN2A1 buried 477 Å² (BSA of total interface=945 Å²) (FIG. 1G), which is approximately half of a typical αβTCR-pMHC complex, with the molecules anchored together by fourteen H-bonds or salt bridges (Table 4).

TABLE 4
Vγ9 TCR contacts with BTN2A1.

| Vγ9 TCR | Vγ9 TCR | BTN2A1 | BTN2A1 | Bond |
|---|---|---|---|---|
| region | residue | IgV region | residue | type |
| A-strand | Lys13γ-Nζ | G-strand | Tyr105-OH | HB |
| A-strand | Lys13γ | G-strand | Tyr105 | VDW |
| B-strand | Ser16γ-O | F-strand | Arg96-Nη2 | HB |
| B-strand | Ser16γ-O | G-strand | Glu107-Oε2 | HB |
| B-strand | Ser16γ | F/G strands | Arg96, Glu107, | VDW |
| | | | Ile109 | |
| B-strand | Lys17γ-Nζ | G-strand | Asp106-Oδ1, | SB |
| | | | Asp106-Oδ2 | |
| B-strand | Lys17γ-Nζ | G-strand | Glu107-O | HB |
| B-strand | Lys17γ | G-strand | Asp106, Glu107 | VDW |
| B-strand | Thr18γ-N | G-strand | Glu107-Oε1 / | HB |
| | | | Glu107-Oε2 | |
| B-strand | Thr18γ-Oγ1 | G-strand | Glu107-Oε1 | HB |
| B-strand | Thr18γ | C/F/G- | Phe39, Arg96, | VDW |
| | | strands | Tyr105, Glu107 | |
| B-strand | Ala19γ | G-strand | Tyr105 | VDW |
| B-strand | Arg20γ-N | G-strand | Tyr105-OH | HB |
| B-strand | Arg20γ-O | G-strand | Tyr105-OH | HB |
| B-strand | Arg20γ-Nε | F-strand | Gln100-Nε2 | Water- |
| | | | | mediated HB |
| B-strand | Arg20γ | CC′-loop, | Phe43, Tyr98, | VDW |
| | | F/G-strands | Tyr105 | |
| B-strand | Arg20γ | CC′-loop | Phe43 | Cation-π |
| C″-strand | Gly67γ-O | CC′-loop | Ser41-O | Water- |
| | | | | mediated HB |
| C″-strand | Gly67γ | CC′-loop | Gln42 | VDW |
| D-strand | Glu70γ-Oε1 | CC′-loop | Phe43-N | HB |
| D-strand | Glu70γ-Oε2 | CC′-loop | Ser44-N | HB |
| D-strand | Glu70γ | CC′-loop | Gln42, Phe43, | VDW |
| | | | Ser44 | |
| E-strand | Thr83γ | CC′-loop | Phe43 | VDW |
| E-strand | His85γ | CC′-loop | Ser41, Gln42, | VDW |
| | | | Phe43 | |
| E-strand | Asn86 | F-strand | Arg96 | VDW |

Van der Waals (VDW) defined as non-hydrogen bond contact distances of 4 Å or less, hydrogen bonds (HB) 3.5 Å or less, cation-π as and salt bridge (SB) as 4.5 Å or less and water-mediated HB 3.3 Å or less.

On the γδTCR, the B-, D- and E-strands of Vγ9 contributed 57%, 17% and 11% of the BSA, respectively, whereas the CC′-loop, F- and G-strands of BTN2A1 contributed 35%, 15%, and 44%, respectively. Within the BTN2A1 interface, the aromatic residues Phe43, Tyr98 and Tyr105 made energetic contributions, with minimal conformational change between the apo and liganded states, indicative of a lock-and-key mode of binding (FIG. 1H). Ser41, Gln42, Phe43 and Ser44 formed the CC′ loop (FIG. 1F), and their involvement is consistent with the overrepresentation of this loop in other IgV-mediated interfaces (S. V. Kundapura, U. A. Ramagopal, The CC′ loop of IgV domains of the immune checkpoint receptors, plays a key role in receptor:ligand affinity modulation. Sci Rep 9, 19191 (2019)). Of note, the aromatic side chain of Phe43 sat planar to the guanidinium moiety of the Arg20γ side chain (FIG. 2E), facilitating a cation-π interaction with a predicted electrostatic binding energy of −4.6 kcal/mol.

Arg20γ also formed a water-mediated H-bond with Gln100 of BTN2A1, along with main chain-mediated H-bonds to the Tyr105 side chain hydroxyl group (FIG. 2E), providing a structural basis for the importance of Arg20γ in BTN2A1-binding and pAg reactivity (M. Rigau et al., Butyrophilin 2A1 is essential for phosphoantigen reactivity by gammadelta T cells. Science 367, (2020)). Likewise, mutations to Glu70γ and His85γ abrogate BTN2A1 reactivity (M. Rigau et al., Butyrophilin 2A1 is essential for phosphoantigen reactivity by gammadelta T cells. Science 367, (2020)), and these were connected by an intrachain H-bond, and also bound BTN2A1, with Glu70γ H-bonding to the Phe43 and Ser44 main chains, and His85γ making Van der Waal (VDW) contacts with Ser41, Gln42 and Phe43 on BTN2A1 (FIG. 2F). Further contacts were made by Lys13γ within the A-strand of Vγ9, which H-bonded to Tyr105, and Lys17γ within the B-strand of Vγ9 forming a salt bridge with Asp106 (FIG. 2G). The adjacent Thr18γ H-bonded with Glu107, and Ser16γ H-bonded to the Arg96 side chain (FIG. 2H). Accordingly, BTN engagement by γδTCR represents a fundamentally unique mode of ligand recognition by the immune system.

### BTN3A1 Modulates Vγ9Vδ2⁺ TCR Tetramer Reactivity

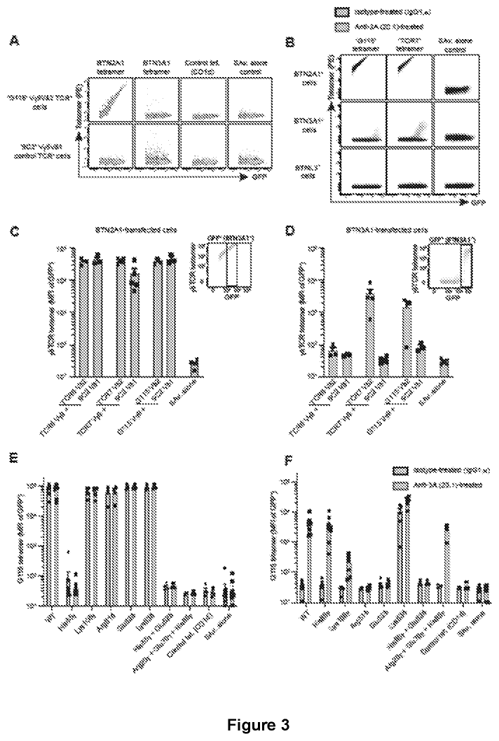

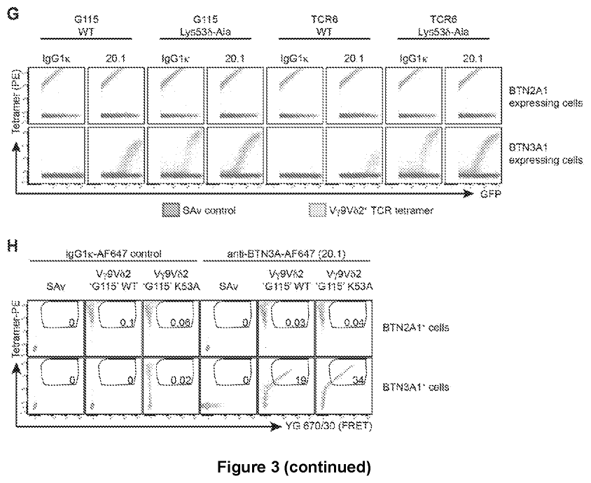

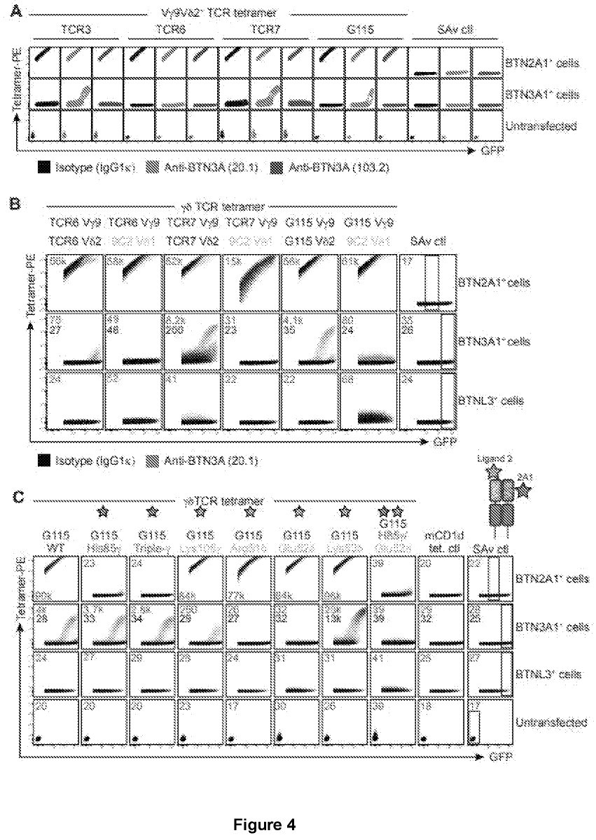

Given the high bioavailability of the apical surface of the Vγ9Vδ2⁺ TCR when liganded to BTN2A1, the present inventors hypothesized that Vγ9Vδ2⁺ TCR co-binds a second ligand. Since BTN3A1 intracellular domain binds pAg (C. Harly et al., Key implication of CD277/butyrophilin-3 (BTN3A) in cellular stress sensing by a major human gammadelta T-cell subset. Blood 120, 2269-2279 (2012); A. Sandstrom et al., The intracellular B30.2 domain of butyrophilin 3A1 binds phosphoantigens to mediate activation of human Vgamma9Vdelta2 T cells. Immunity 40, 490-500 (2014)), they first examined whether soluble BTN3A1 ectodomain could directly bind γδTCR by probing Vγ9Vδ2⁺ TCR-transfected BTN2A^{KO}.BTN3A^{KO} HEK293T cells, which lack endogenous BTN2A1, 2A2, 2A3p, 3A1, 3A2 and 3A3, with BTN3A1 ectodomain tetramers. Consistent with an earlier report (A. Sandstrom et al., The intracellular B30.2 domain of butyrophilin 3A1 binds phosphoantigens to mediate activation of human Vgamma9Vdelta2 T cells. Immunity 40, 490-500 (2014)), they did not detect any reactivity (FIG. 3A). The present inventors next tested whether mouse NIH-3T3 fibroblasts, which lack human BTN or BTNL molecules and are inherently incapable of mediating Vγ9Vδ2⁺ T cell activation by pAg, that were transfected with full-length human BTN3A1, could bind Vγ9Vδ2⁺ TCR tetramer. Compared to BTN2A1⁺ NIH-3T3 cells, which bound all Vγ9Vδ2⁺ TCR tetramers (clones TCR3, TCR6, TCR7 and G115), BTN3A1⁺ cells showed little, if any, staining (FIG. 4A, black plots). Previous studies showed that crosslinking of BTN3A1 on the surface of APCs with anti-BTN3A antibody (mAb clone 20.1) converts BTN3A1 into a stimulatory form that can activate Vγ9Vδ2⁺ T cells, in a way that mimics pAg challenge (14, 15). Conversely, a separate anti-BTN3A mAb (clone 103.2) is a potent antagonist of Vγ9Vδ2⁺ T cell reactivity to pAg (14, 15). Strikingly, cross-linking of BTN3A1⁺ cells with agonistic mAb clone 20.1 induced clear staining of Vγ9Vδ2⁺ TCR tetramers, particularly clones TCR3, TCR7 and G115 (FIG. 4A, light grey plots). This contrasted the antagonist anti-BTN3A mAb clone 103.2, which did not induce any Vγ9Vδ2⁺ TCR tetramer staining, nor did mAb 20.1 treatment of untransfected or BTNL3-transfected cells (FIG. 4A, dark grey plots; and 4B). The present inventors obtained a similar pattern of mAb 20.1-induced BTN3A1-dependent Vγ9Vδ2⁺ TCR staining using BTN3A1-transfected human BTN2A^{KO}.BTN3A^{KO} HEK293T cells (FIG. 3B). Furthermore, chimeric TCR tetramers comprised of a pAg-reactive Vγ9⁺γ-chain paired with an irrelevant Vδ1+6-chain retained reactivity to BTN2A1⁺ cells, but not to mAb 20.1-cross-linked BTN3A1⁺ cells, indicating that unlike BTN2A1 reactivity, BTN3A1 reactivity depends on Vδ2 and/or the CDR36 loops (FIG. 4B; FIG. 3C and D). Thus, mAb 20.1 pre-treatment of BTN3A1-transfected cells induces reactivity to Vγ9Vδ2⁺ TCR via recognition of a second ligand, herein termed ‘ligand-two’. Ligand-two reactivity could be induced upon mAb 20.1 cross-linking of BTN3A1 in both human and mouse cell lines, and unlike BTN2A1 reactivity, this binding appeared to depend on the Vδ2 domain and/or the CDR3 loops, hereafter referred to as ‘epitope two’ (FIG. 4C, cartoon inset).

Lys53δ Regulates the Interaction with Ligand-Two

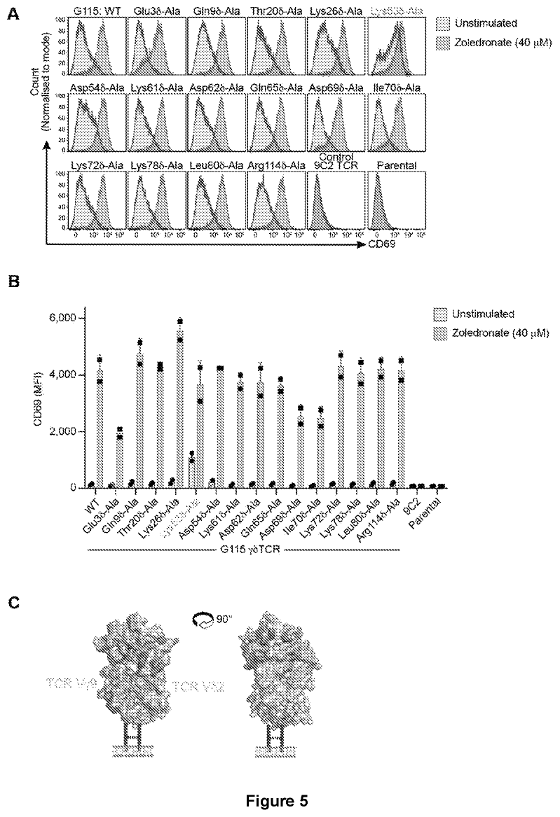

Since the ABED β-sheet of Vγ9 mediates binding to BTN2A1 (FIG. 2C), the present inventors tested whether the symmetrically equivalent ABED β-sheet of Vδ2 is also important in sensing pAg. Jurkat cells expressing Ala mutants within the Vδ2-encoded ABED β-sheet did not impair reactivity to zoledronate (an aminobisphosphonate that increases intracellular IPP pAg), suggesting there is no Vδ2-encoded equivalent ABED binding interface to the BTN2A1-binding domain on Vγ9 (FIGS. 5A, B and C). Since pAg-mediated γδ T cell responses depend on Arg51 δ and Glu52δ, both located within the CDR26 loop ((10, 18) and FIG. 5C, grey residues), the present inventors screened two additional mutations within this loop: Lys53δ-Ala and Asp54δ-Ala. Whilst Asp54δ-Ala did not appreciably affect reactivity to zoledronate, Jurkat cells expressing the G115 Vγ9Vδ2⁺ TCR with a Lys53δ-Ala mutation exhibited spontaneous activation, indicating that this residue may have a role in dampening γδ T cell reactivity to TCR stimuli (FIGS. 5A, B and C).

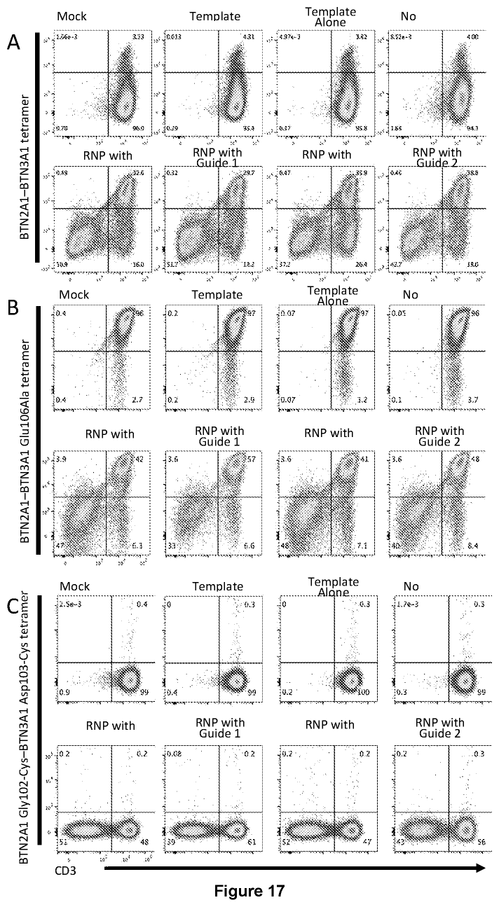

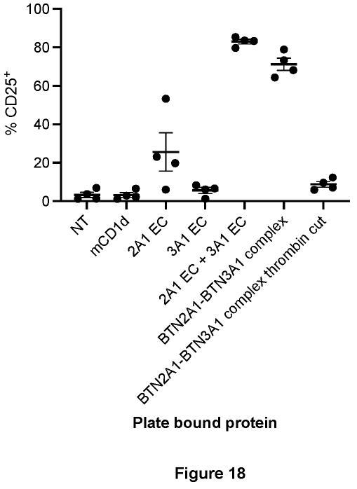

To reconcile these observations with mAb 20.1-induced Vγ9Vδ2⁺ TCR tetramer staining of BTN3A1⁺ cells, the present inventors produced G115 Vγ9Vδ2⁺ TCR tetramer (hereafter referred to as G115 tetramer) with the corresponding Ala substitutions. As expected, wild-type G115 tetramer interacted with BTN2A1⁺ NIH-3T3 fibroblasts, and also with mAb 20.1-pretreated BTN3A1⁺ cells (FIG. 4C; FIGS. 3E and F). G115 tetramers with mutations at the BTN2A1 binding site (‘epitope one’), notably His85γ-Ala and an Arg20γ-Ala/Glu70γ-Ala/His85γ-Ala triple-mutant, were unable to stain BTN2A1⁺ cells, but still retained the ability to interact with mAb 20.1-pretreated BTN3A1⁺ cells (FIG. 4C; FIGS. 3E and F). Conversely, G115 tetramers with ‘epitope two’ Arg51 5-Ala or Glu52δ-Ala mutations readily stained BTN2A1⁺ cells, but lost their ability to react with mAb 20.1-pretreated BTN3A1⁺ cells (FIG. 4C; FIGS. 3E and F). Lys108γ-Ala, located within the CDR3γ and near the CDR26 (5-8 Å away), also exhibited a reduced association with mAb 20.1-pretreated BTN3A1⁺ cells, but not to BTN2A1 (FIG. 4C; FIGS. 3E and F). Strikingly, G115 tetramers with a Lys53δ-Ala substitution, which was the mutant that resulted in autoactivation in functional assays (FIGS. 5A and B), did not affect reactivity to BTN2A1⁺ cells, but stained BTN3A1⁺ cells even without any mAb 20.1 cross-linking (FIG. 4C; FIGS. 3E and F). Indeed, mAb 20.1 pre-treatment only marginally enhanced Lys53δ-Ala G115 tetramer reactivity to BTN3A1⁺ cells above this spontaneous level of interaction (FIGS. 4C and 3F). The strong interaction of G115 γδTCR tetramers that contained a Lys53δ-Ala substitution with BTN3A1⁺ cells also held true for other Vγ9Vδ2⁺ TCR clones tested (FIG. 3G), indicating that the Lys53δ-Ala mutation enhances Vγ9Vδ2⁺ TCR binding potential irrespective of CDR3 sequence heterogeneity. This was further demonstrated by genetic modification of Lys53δ-Ala in polyclonal Vγ9Vδ2⁺ T cells, which resulted in enhancement of binding to BTN2A1-BTN3A1 heteromers by the majority of Vγ9Vδ2⁺ cells (FIG. 17A). G115 tetramers with combined His85γ-Ala (in epitope one) and Glu52δ-Ala (in epitope two) mutations lost the ability to interact with both BTN2A1⁺ and also mAb 20.1-pretreated BTN3A1⁺ cells (FIG. 4C; FIGS. 3E and F). In further support of the observation that Vγ9Vδ2⁺ TCR closely associates with BTN3A1 following anti-BTN3A mAb 20.1-pretreatment, we co-stained BTN3A1- or BTN2A1-expressing cells with control SAv-PE or Vγ9Vδ2 TCR-PE tetramer, along with isotype control-AF647 (MOPC21) or anti-BTN3A-AF647 (20.1) mAb (FIG. 3H). Förster resonance energy transfer (FRET) was observed when BTN3A1⁺ cells were co-stained with Vγ9Vδ2 TCR-PE tetramer and anti-BTN3A-AF647 Ab, suggesting close proximity (<10 nm) when co-bound to BTN3A1-transfected cells. Collectively, these data suggest ‘ligand-two’, being either BTN3A1 itself or a closely associated molecule, binds to Vγ9Vδ2⁺ TCR via ‘epitope two’, located on the apical surface of the Vγ9Vδ2⁺ TCR and incorporating residues within the CDR2δ and CDR3γ loops. Within epitope two, Lys53δ appears to act as a gatekeeper residue for ligand-two accessibility, suggesting that upon cross-linking of BTN3A1 with agonist mAb 20.1, a conformational change to ligand-two occurs that partly circumvents this steric barrier.

### BTN3A1 is a Direct Ligand of the Vγ9Vδ2⁺ TCR.

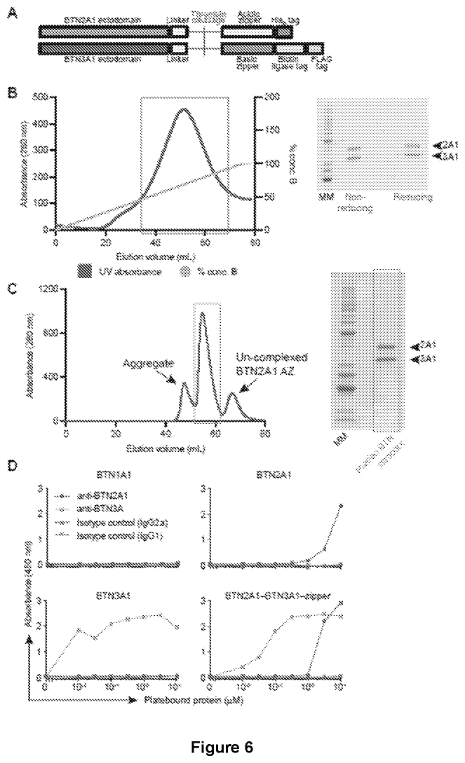

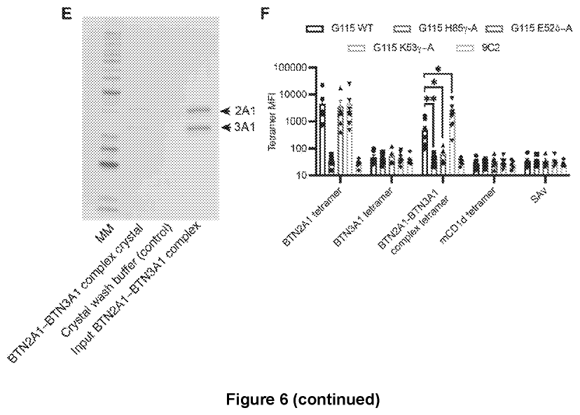

The present inventors next explored the hypothesis that ligand-two is BTN3A1, and that BTN2A1 stabilizes BTN3A1 binding to the γδTCR. To test this, the present inventors produced soluble BTN3A1-BTN2A1 ectodomain heteromeric complexes (FIG. 6A), which were tethered together with C-terminal leucine zippers, and measured whether they could bind to epitope two, being the ligand-two binding site on Vγ9Vδ2⁺ TCR. The BTN2A1-BTN3A1 heteromer complex retained staining with anti-BTN2A1 and anti-BTN3A1 mAb by ELISA (FIG. 6D) and was comprised of two chains after purification (BTN2A1 and BTN3A1; FIG. 6B-C) and following crystallisation (FIG. 6E), suggestive of a correct conformation.

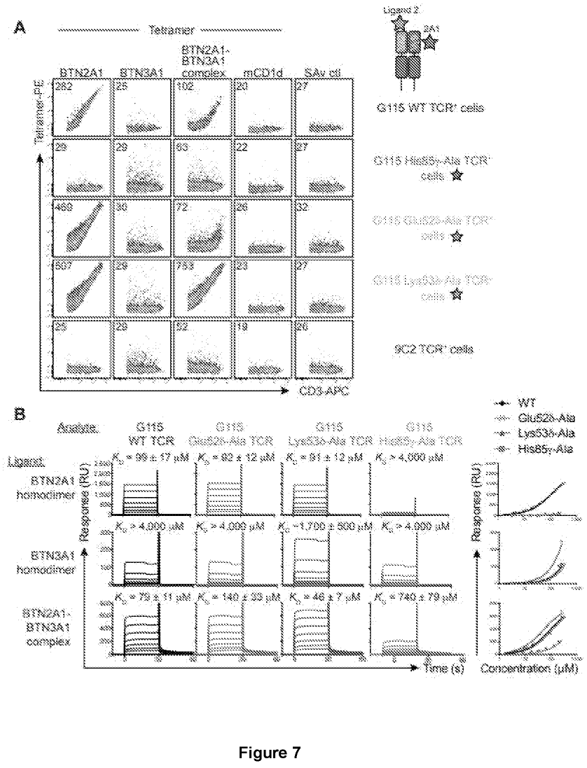

Consistent with the BTN2A1-γδTCR docking mode (FIG. 2C), BTN2A1 tetramers readily stained G115 TCR⁺ WT cells, as did G115 mutants located in epitope two, namely Glu52δ-Ala and Lys53δ-Ala, but not the epitope one mutant His85γ-Ala (FIG. 7A and FIG. 6F). Soluble BTN3A1 ectodomain tetramers failed to interact with G115 TCR⁺ WT HEK-293T cells (FIG. 7A and (A. Sandstrom et al., The intracellular B30.2 domain of butyrophilin 3A1 binds phosphoantigens to mediate activation of human Vgamma9Vdelta2 T cells. Immunity 40, 490-500 (2014)). BTN2A1-BTN3A1 complex tetramers also bound G115 TCR⁺ WT cells, but at slightly lower levels than BTN2A1 tetramers (FIG. 7A and FIG. 6F). Akin to BTN2A1 tetramers, a His85γ-Ala mutation completely abrogated the interaction with BTN2A1-BTN3A1 tetramers, indicating a strong dependence on BTN2A1. However, unlike BTN2A1 tetramers, BTN2A1-BTN3A1 tetramer binding was heavily modulated by mutations to epitope two. Here, Glu52δ-Ala, which was essential for G115 tetramer staining of BTN3A1-transfected cells (FIG. 4C), marginally reduced reactivity to BTN2A1-BTN3A1, compared to G115 WT TCR, whereas the gatekeeper residue mutant Lys53δ-Ala resulted in a clear increase in BTN2A1-BTN3A1 staining intensity (FIG. 7A and FIG. 6F). These data indicate that soluble BTN2A1-BTN3A1 ectodomain complex can bind γδTCR, but, unlike BTN2A1 alone, BTN2A1-BTN3A1 complex binding is co-dependent on epitopes one and two.

The present inventors next tested whether BTN2A1-BTN3A1 complexes can co-bind epitopes one and two of Vγ9Vδ2⁺ TCR in a cell-free assay, by using surface plasmon resonance (FIG. 7B). Soluble G115 WT γδTCR bound immobilized BTN2A1 homodimer with an affinity of K_{D}=99 μM, which is similar to previous studies (K_{D}=40-50 μM in (M. Rigau et al., Butyrophilin 2A1 is essential for phosphoantigen reactivity by gammadelta T cells. Science 367, (2020); M. M. Karunakaran et al., Butyrophilin-2A1 Directly Binds Germline-Encoded Regions of the Vgamma9Vdelta2 TCR and Is Essential for Phosphoantigen Sensing. Immunity 52, 487-498 e486 (2020)), but did not bind immobilized BTN3A1 (K_{D}>4,000 μM). Consistent with the role of epitope one, but not epitope two, in binding BTN2A1, soluble G115 TCR with a His85γ-Ala substitution abrogated reactivity to BTN2A1, whereas Glu52δ-Ala and Lys53δ-Ala had no effect. Interestingly, the gatekeeper mutant Lys53δ-Ala exhibited some low-level binding to BTN3A1 at the highest concentrations, but the predicted affinity was very weak (K_{D}˜1,700 μM). G115 WT γδTCR bound immobilized BTN2A1-BTN3A1 complex with a similar affinity to BTN2A1 (K_{D}=79 μM and 99 μM, respectively). However, in contrast to G115 TCR binding to BTN2A1, binding of G115 TCR to BTN2A1-BTN3A1 was modulated by mutations within epitope two of the γδTCR, since Lys53δ-Ala resulted in an increase in affinity (K_{D}=46 μM) whereas Glu52δ-Ala resulted in a slight decrease in affinity (K_{D}=140 μM). Furthermore, unlike BTN2A1, the BTN2A1-BTN3A1 complex also reacted weakly to G115 His85γ-Ala TCR (K_{D}=740 μM; FIG. 7B). Therefore, the pattern of ligand-two reactivity to Vγ9Vδ2⁺ TCR can be recapitulated with soluble BTN2A1-BTN3A1 complex, in both cell-surface staining-based and cell-free biophysical-based assays. Together, these data reveal that BTN3A1, along with BTN2A1, are necessary and sufficient to co-engage γδTCR via epitopes one and two.

BTN3A1 IgV Domain Interacts with Both BTN2A1 and Vγ9Vδ2⁺ TCR

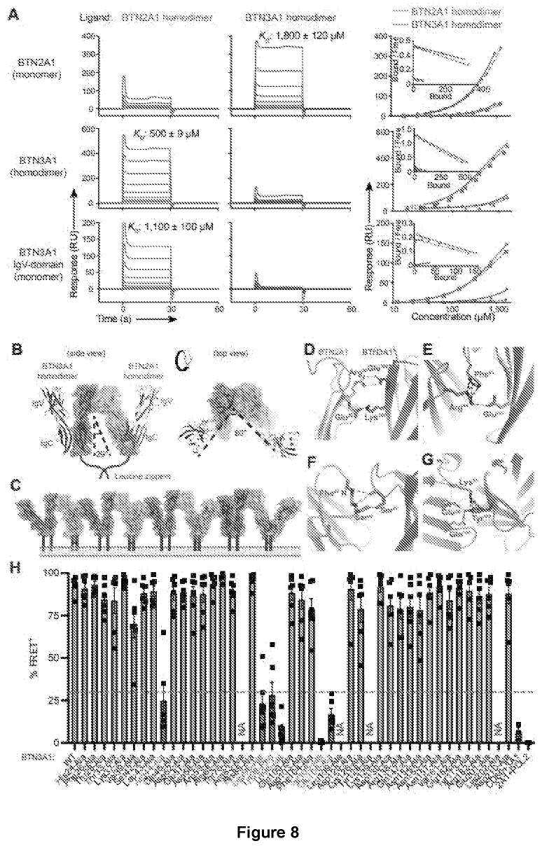

BTN2A1 and BTN3A1 are located within 10 nm of each other in cis on the cell surface (M. Rigau et al., Butyrophilin 2A1 is essential for phosphoantigen reactivity by gammadelta T cells. Science 367, (2020)), however, whether they directly interact is unclear. Using surface plasmon resonance, full-length BTN3A1 ectodomain (IgV-IgC) bound immobilized disulfide-linked BTN2A1 homodimer with an affinity of K_{D}=500 μM, but not immobilized BTN3A1 homodimer. Conversely, full-length BTN2A1 ectodomain weakly bound immobilised BTN3A1 homodimer (K_{D}˜1800 μM), but not immobilized BTN2A1 homodimer (FIG. 8A), indicating that BTN2A1 and BTN3A1 ectodomains are capable of directly interacting, albeit with a low affinity. Since BTN3A1 ectodomain exists as a homodimer and may therefore exhibit enhanced binding in SPR assays due to increased avidity, the present inventors also tested monomeric BTN3A1 IgV domain, which retained specific binding to BTN2A1 (K_{D}=1,100 μM; FIG. 8A). To understand the molecular nature of this interaction, the present inventors crystallized the BTN2A1-BTN3A1-zipper complex ectodomains.

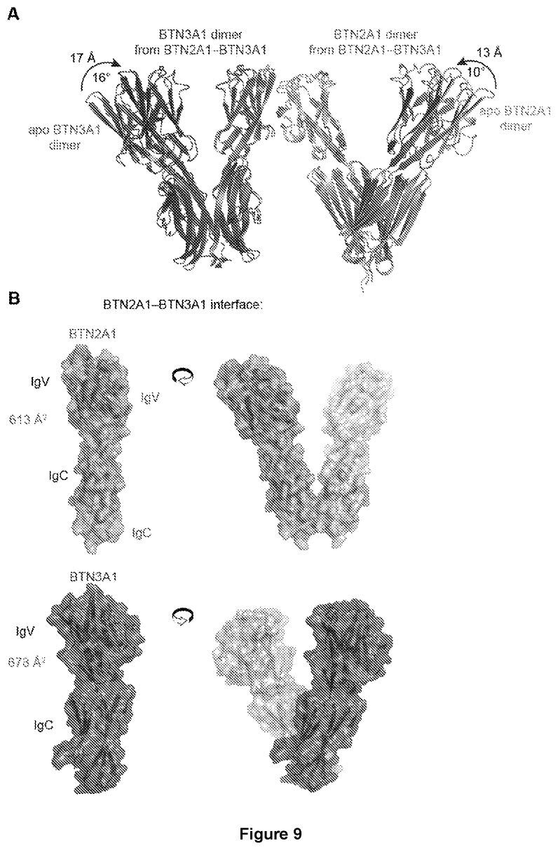

The crystals diffracted anisotropically to 5.6 Å resolution, with a single copy each of BTN2A1 and BTN3A1 monomer in the asymmetric unit, which interfaced via their IgV domains at a docking angle of ˜29° (FIG. 8B and Table 1). The C-terminal zipper domains were mobile and not modelled, although there was space for them within the asymmetric unit underneath the IgC domains. The V-shaped homodimers of both BTN2A1 and BTN3A1 were also present within the crystallographic symmetry (FIG. 8B), although were twisted by ˜10° and ˜16° compared to the apo V-dimers, respectively (FIG. 9A). The BTN2A1 and BTN3A1 V-dimers buried 613 Å² and 678 Å², respectively, for a combined BSA of ˜1,300 Å². The BTN2A1 and BTN3A1 V-dimers came together at a planar angle of ˜80° to form a distorted W-shaped heterotetramer (FIG. 8B), which could be even further expanded through crystallographic symmetry to yield a linear polymer of the composition [BTN2A1_{homodimer}-BTN3A1_{homodimer}]ₙ (FIG. 8C).

The BTN2A1-BTN3A1 intermolecular contacts were determined based on a model wherein higher resolution apo BTN2A1 and BTN3A1 structures were fitted into the low-resolution complex electron density map. Assuming no significant side-chain movements, a network of intermolecular salt bridges were present, including BTN2A1-Arg56 to BTN3A1-GluIgV106, BTN2A1-Glu35 to BTN3A1-Lys107, BTN2A1-Glu62 to BTN3A1-Lys94, and BTN2A1-Glu107 to BTN3A1-Arg44 (FIG. 8D and E; Table 5). Furthermore, BTN2A1-Phe43, which formed a cation-π interface with Arg20 of the TCR γ-chain in the BTN2A1-γδTCR structure (FIG. 2E), also formed a cation-π interface with the Arg44 side chain of BTN3A1 (electrostatic binding energy of −4.7 kcal/mol; FIG. 8E). There were seven additional H-bonds, mostly mediated by the C-strand, CC′-loop and C′-strands of BTN2A1 (Table 4), including BTN2A1-Ser44 N and O atoms, which contacted Ser41-O and Oγ, respectively (FIG. 8F). Tyr105 of BTN3A1 also made extensive contacts with BTN2A1, including a cation-π interface with the terminal amine of BTN2A1-Lys51 (binding energy of −5.4 kcal/mol), along with H-bonds to the BTN2A1-Glu35 and Gln00 sidechains (FIG. 8G).

TABLE 5
BTN2A1 ectodomain contacts with BTN3A1 ectodomain.

| BTN2A1 | BTN2A1 | BTN3A1 | BTN3A1 | Bond |
|---|---|---|---|---|
| IgV region | residues | IgV region | residues | type |
| C-strand | Glu35-Oε1 | G-strand | Tyr105-OH | HB |
| C-strand | Glu35-Oε1, | G-strand | Lys107-Nζ | SB |
| | Oε2 | | | |
| C-strand | Glu35 | G-strand | Tyr105, Lys107 | VDW |
| C-strand | Arg37 | G-strand | Lys107 | VDW |
| C-strand | Ser41 | C-strand | Ser42 | VDW |
| CC′-loop | Gln42-Oε1 | C-strand | Ser42-Oγ | HB |
| CC′-loop | Gln42 | C/F-strands | Ser41, Ser42, | VDW |
| | | | Lys94 | |
| CC′-loop | Phe43-N | C-strand | Ser41-O | HB |
| CC′-loop | Phe43 | CC′-loop | Arg44 | Cation-π |
| CC′-loop | Phe43 | C-strand | Ser41, Ser42, | VDW |
| | | CC′-loop | Arg44 | |
| CC′-loop | Ser44-N | C-strand | Ser41-O | HB |
| CC′-loop | Ser44-O | C-strand | Ser41-Oγ | HB |
| CC′-loop | Ser44 | C/G-strands | Ser41, Leu109 | VDW |
| CC′-loop | Pro45 | F-strand | Lys94 | VDW |
| C′-strand | Lys51-Nζ | G-strand | Tyr105-O | HB |
| C′-strand | Lys51 | G-strand | Tyr105, Glu106, | VDW |
| | | | Lys107 | |
| C′-strand | Lys51 | G-strand | Tyr105 | Cation-π |
| C″-strand | Arg56-Nη1, | G-strand | Glu106-Oε1, | SB |
| | Nη2 | | Oε2 | |
| C″-strand | Arg56 | G-strand | Glu106 | VDW |
| C″-strand | Glu58 | G-strand | Leu109 | VDW |
| C″-strand | Glu59 | F/G-strands | Leu96, Lys107, | VDW |
| | | | Leu109 | |
| C″D-loop | Glu62-Oε2 | F-strand | Lys94-Nζ | SB |
| F-strand | Gln100-Oε1 | G-strand | Tyr105-OH | HB |
| F-strand | Gln100 | G-strand | Tyr105 | VDW |
| FG-loop | Gly102 | G-strand | Tyr105 | VDW |
| FG-loop | Arg103 | F-strand | Gln100 | VDW |
| G-strand | Glu107-Oε1 | CC′-loop | Arg44-Nη1 | SB |

Van der Waals (VDW) defined as non-hydrogen bond contact distances of 4 Å or less, hydrogen bonds (HB) as 3.5 A or less, salt bridge (SB) as 4.5 Å or less and cation-π as 4.5 Å or less.

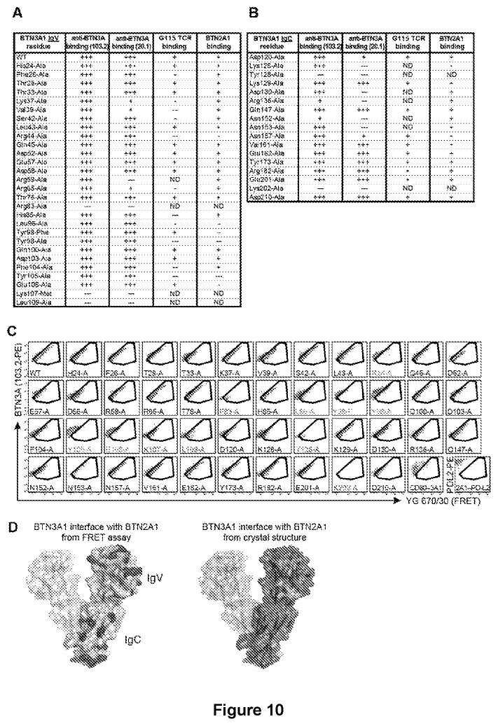

The present inventors next tested whether the same BTN3A1 residues that engaged BTN2A1 in the W-shaped complex correlated with those responsible for the reported cis association between BTN2A1 and BTN3A1 on the cell surface (M. Rigau et al., Butyrophilin 2A1 is essential for phosphoantigen reactivity by gammadelta T cells. Science 367, (2020)). Of a panel of forty-five BTN3A1 Ala ectodomain mutants, including residues within both the IgV and IgC domains, forty retained expression on the cell surface and reactivity to anti-BTN3A mAb clone 103.2 (FIGS. 10A and B). Mutations to five residues: Arg44-Ala, Leu96-Ala, Tyr98-Ala (and additionally Tyr98-Phe), Tyr105-Ala and Glu106-Ala abrogated Förster resonance energy transfer (FRET) between anti-BTN2 Å and anti-BTN3A mAbs (FIG. 8H and FIG. 10C). These residues mapped to the CFG face of BTN3A1 and correlated closely with the crystal structure interface (FIG. 10D), thereby validating this mode of binding. Thus, BTN2A1 and BTN3A1 interact via the CFG faces of their IgV domains and form W-shaped heterodimers and/or hetero-oligomers.

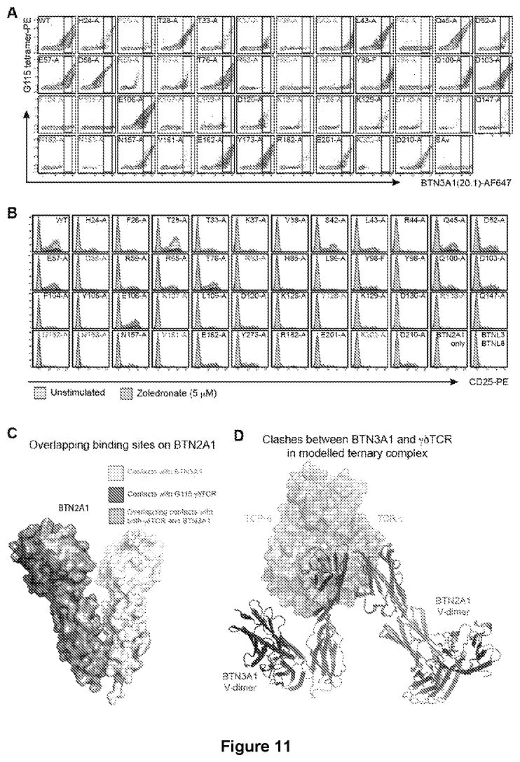

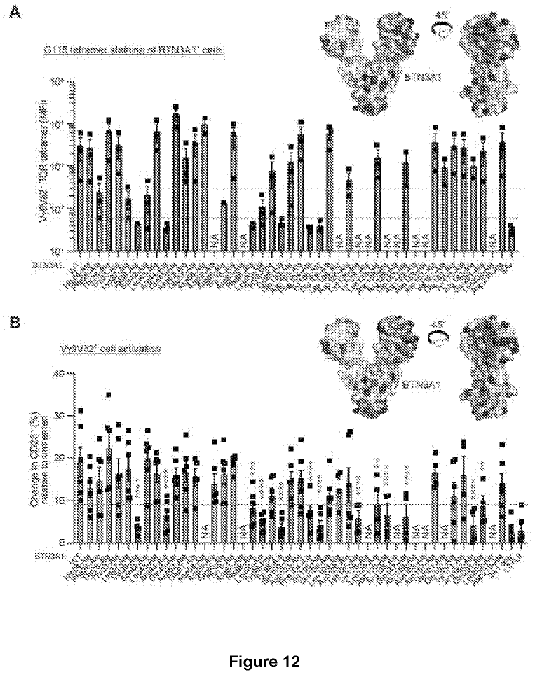

Using this panel of BTN3A1 Ala mutants, the present inventors investigated which residues were involved in the Vγ9Vδ2⁺ TCR interaction. Thirty-four of the panel of forty-five mutants retained reactivity to anti-BTN3A mAb clone 20.1 mAb (FIGS. 10A and 11A). Of these, six completely abrogated G115 tetramer staining of mAb 20.1-pretreated BTN3A1⁺ cells: Val39, Arg44, His85, Tyr98, Phe104 and Tyr105, plus a further four residues that reduced G115 tetramer staining by >90%: Phe26, Lys37, Ser42 and Leu96 (FIGS. 12A and 11A). The panel of BTN3A1 Ala mutants were next co-expressed with BTN2A1 (WT) in NIH-3T3 cells and used to activate Vδ2⁺ T cells in the presence of zoledronate. All six BTN3A1 residue Ala mutants that abrogated G115 tetramer reactivity—Val39, Arg44, His85, Tyr98, Phe104 and Tyr105—also abrogated Vδ2⁺ T cell activation, as did Leu96 (FIG. 12B and FIG. 11B). Except for His85, which mapped to the ABED face, all other residues mapped to the CFG face. These data extend upon an earlier report that the CFG face of BTN3A1 IgV domain is functionally important (C. R. Willcox et al., Butyrophilin-like 3 Directly Binds a Human Vgamma4(+) T Cell Receptor Using a Modality Distinct from Clonally-Restricted Antigen. Immunity 51, 813-825 e814 (2019)), and moreover, attribute a role for these residues in binding to Vγ9Vδ2⁺ TCR.

BTN2A1 and BTN3A1 utilize the same epitopes to bind each other and Vγ9Vδ2⁺ TCR

Paradoxically, four of the seven BTN3A1 residues Ala mutants (Arg44, Leu96, Tyr98 and Tyr105) that were important for binding to Vγ9Vδ2⁺ TCR were also critical for binding to BTN2A1 (FIGS. 8H, 9A, and 10D). Likewise, many of the residues within the BTN2A1 IgV domain that contacted γδTCR also mediated binding to BTN3A1, including Phe43, Ser44 and Glu107 (Tables 4 and 5; FIG. 11C). The overlap between the BTN2A1 and γδTCR-binding domains on BTN3A1, and conversely, between the BTN3A1 and γδTCR-binding domains on BTN2A1, raised the key question of how BTN2A1 and BTN3A1 can co-bind to each other, and Vγ9Vδ2⁺ TCR, at the same time. Indeed, a superimposition of the BTN2A1-γδTCR and BTN2A1-BTN3A1 crystal structures identified major steric clashes between BTN3A1 and the γδTCR (>15 Å), suggesting that co-binding as a ternary complex in this manner is impossible (FIG. 11D). This implies that whilst both BTN2A1 and BTN3A1 are co-ligands for each other, they must disengage or undergo major conformational changes prior to co-binding Vγ9Vδ2⁺ TCR.

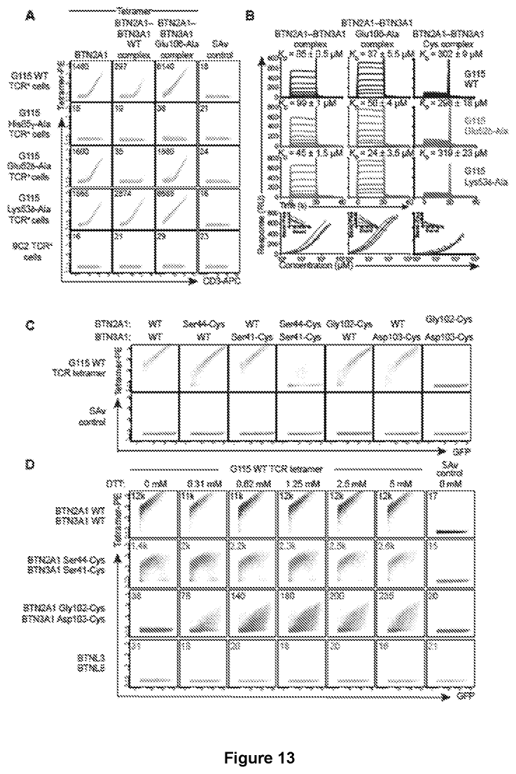

The present inventors tested this hypothesis using BTN2A1-BTN3A1-zipper ectodomain complex tetramers that contained the BTN3A1 Glu106-Ala mutant, which, based on FRET measurements, largely disrupts the BTN2A1-BTN3A1 ectodomain complex but without having any detrimental effect on G115 tetramer reactivity to BTN3A1 (FIGS. 8H and 12A respectively). Compared to BTN2A1-BTN3A1 WT tetramers, BTN2A1 WT-BTN3A1 Glu106-Ala tetramers stained G115 WT γδTCR-transfected HEK-293T cells at a higher intensity, indicating that the affinity may be increased (FIG. 13A). The Glu525-Ala G115 γδTCR mutant, which abrogates binding to BTN3A1, was also stained more strongly by the BTN2A1-BTN3A1 Glu106-Ala tetramers, suggesting that binding to the BTN2A1 ‘epitope one’ on γδTCR is enhanced by the BTN3A1 Glu106-Ala mutation. The BTN2A1 WT-BTN3A1 Glu106-Ala tetramers stained G115 WT, but not G115 Glu52δ-Ala γδTCR⁺ cells, more brightly than BTN2A1 tetramers, further suggesting that BTN2A1 WT-BTN3A1 Glu106-Ala complexes may exhibit even higher affinity than BTN2A1 tetramers. To directly measure the binding affinity, the present inventors performed SPR (FIG. 13B). Consistent with the tetramer staining of cell lines, G115 WT γδTCR bound immobilized BTN2A1-BTN3A1 Glu106-Ala complexes with a higher affinity than BTN2A1-BTN3A1 WT complexes (K_{D}=37 μM compared to 85 μM, respectively), suggesting that the BTN3A1 Glu106-Ala mutation enhances accessibility to Vγ9Vδ2⁺ TCR. The affinity of γδTCR to BTN2A1-BTN3A1 Glu106-Ala complexes was higher than the G115 γδTCR-BTN2A1 interaction (K_{D}=37 μM compared to 99 μM), supporting a model wherein the presence of both BTN ligands in an accessible conformation results in enhanced affinity, mediated by a simultaneous co-binding event. Therefore, BTN2A1 and BTN3A1 each contain epitopes that are reactive to separate determinants on Vγ9Vδ2⁺ TCR, and these BTN epitopes are tethered to each other on the cell surface, which prevents the TCR from efficiently engaging. Upon a conformational change in BTN3A1, for example as mediated by agonist clone 20.1 mAb, the BTN ectodomains acquire the ability to simultaneously co-bind Vγ9Vδ2⁺ TCR.

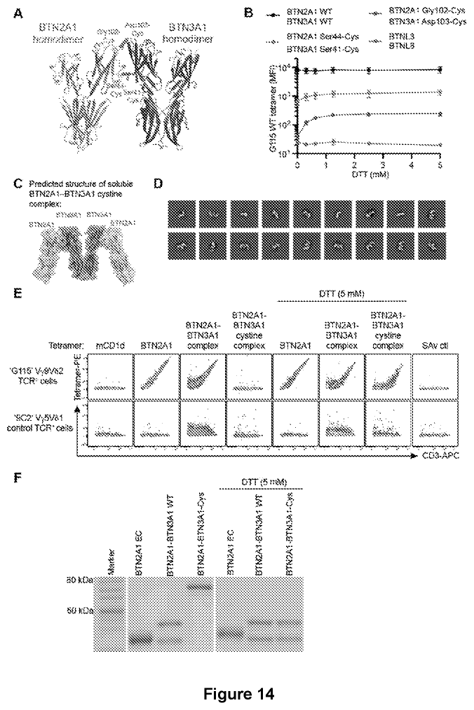

To further test this model, the present inventors reasoned that locking BTN2A1 and BTN3A1 ectodomains together in their W-shaped conformation would abrogate their reactivity to Vγ9Vδ2⁺ TCR. For this, the present inventors introduced cysteine (Cys) residues in the BTN2A1 and BTN3A1 IgV domain CFG faces that were optimally spaced for formation of an interchain disulfide bond. The present inventors identified two separate Cys pairs, using the structure of BTN2A1-BTN3A1 complex as a guide: BTN2A1 Gly102-Cys plus BTN3A1 Asp103-Cys, and BTN2A1 Ser44-Cys plus BTN3A1 Ser41-Cys (FIG. 14A). Cells co-transfected with BTN2A1 Ser44-Cys plus BTN3A1 Ser41-Cys, or BTN2A1 Gly102-Cys plus BTN3A1 Asp103-Cys, exhibited a major reduction or total loss of G115 tetramer reactivity, respectively (FIG. 13C). However, when the Cys mutants were co-expressed with the corresponding WT molecule (for example, BTN2A1 Gly102-Cys plus BTN3A1 WT, or vice versa), their reactivity to Vγ9Vδ2⁺ TCR was retained, indicating that an interchain disulfide bond was responsible for the loss of reactivity to Vγ9Vδ2⁺ TCR (FIG. 13C). In further support of this notion, treatment of BTN2A1-Cys⁺ BTN3A1-Cys⁺ cells with graded doses of the reducing agent dithiothreitol (DTT) partly restored the ability of G115 tetramer to stain these cells (FIG. 13D and FIG. 14B).

Based on the BTN2A1-BTN3A1 crystal structure, the present inventors predicted that soluble BTN2A1 Gly102-Cys-BTN3A1 Asp103-Cys ectodomain complexes, with C-terminal zippers removed, would adopt an M-shaped tetramer comprised of a core BTN3A1 V-dimer and two outer copies of BTN2A1, each linked to BTN3A1 via a disulfide bond (FIG. 14C). 2D class averages of negatively stained micrographs of soluble BTN2A1 Gly102-Cys-BTN3A1 Asp103-Cys complex indeed revealed the presence of M-shaped particles, further supporting this notion (FIG. 14D). A fluorescently tagged tetrameric form of the BTN2A1 Gly102-Cys-BTN3A1 Asp103-Cys heteromers failed to stain G115 γδTCR-transfected HEK-293T cells; however, pre-treatment of these soluble tetrameric complexes with DTT immediately prior to staining reduced the disulfide bond and restored their reactivity to G115 γδTCR+ cells (FIGS. 14E and F). Thus, locking BTN2A1 and BTN3A1 together with a covalent disulfide bond prevents engagement by Vγ9Vδ2⁺ TCR, and disruption of this bond restores Vγ9Vδ2⁺ TCR reactivity.

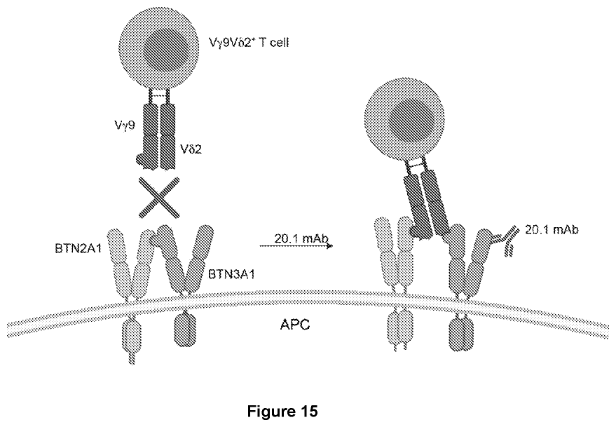

Together, these findings reveal that Vγ9Vδ2⁺ TCR co-binds BTN2A1 and BTN3A1 via two spatially distinct epitopes, with BTN2A1 engaging the side of Vγ9, and BTN3A1 binding to the apical surface. BTN2A1 and BTN3A1 also interact with each other in cis, forming W-shaped multimers, but in doing so, cannot engage γδTCR. The present inventors propose that pAg sequestration by the intracellular domain of BTN3A1 induces remodelling or multimerisation of the intracellular B30.2 domains, which in turn facilitates allosteric changes to the ectodomains, converting them from an inactive ‘cryptic’ state into an active ‘open-altered’ state. The activated BTN2A1-BTN3A1 complexes can react with Vγ9Vδ2⁺ TCR, facilitating γδ T cell-mediated immunity (FIG. 15).

Akin to pMHC recognition by the αβTCR, BTN molecules have emerged as important γδTCR ligands, however, their molecular mode of recognition is poorly defined. Furthermore, the precise mechanism by which Vγ9Vδ2⁺ T cells recognise pAg remains unclear. Here the present inventors report the first structure of a TCR engaging a non-MHC or MHC-like ligand, namely BTN2A1, revealing that BTN2A1 engages the side of Vγ9, leaving the apical face of the Vγ9Vδ2⁺ TCR exposed. The present inventors also demonstrate that a second ligand, BTN3A1, binds the apical Vγ9Vδ2⁺ TCR surface.

The ability of Vγ9Vδ2⁺ TCR to co-bind two ligands contrasts the recognition of MHC and MHC-like molecules by αβ T cells, which bind with one-to-one stoichiometry. Thus, the γδTCR appears to be capable of discriminating between a dual and a single ligand-binding event. Since Vγ9 is often incorporated into non-pAg-reactive Vγ9Vδ1⁺ TCRs, other non-BTN γδ T cell ligands such as MICA, CD1 or MR1 might also co-bind in conjunction with BTN2A1. Likewise, BTNL3 can bind Vγ4⁺ TCRs in a similar manner to Vγ9 and BTN2A1, although whether BTNL8 can also co-bind γδTCR has not been determined (D. Melandri et al., The gammadeltaTCR combines innate immunity with adaptive immunity by utilizing spatially distinct regions for agonist selection and antigen responsiveness. 19, 1352-1365 (2018); C. R. Willcox et al., Butyrophilin-like 3 Directly Binds a Human Vgamma4(+) T Cell Receptor Using a Modality Distinct from Clonally-Restricted Antigen. Immunity 51, 813-825 e814 (2019)).

Unlike αβTCRs and BCRs, which directly sense foreign Ag, pAg-reactive γδ TCRs are activated by inside-out signalling via BTN conformational changes. As such, additional regulatory mechanisms are likely required to maintain γδ T cell self-tolerance. To this end the present inventors identified two important molecular checkpoints, namely Lys53 in the CDR2δ loop of Vγ9Vδ2⁺ TCR, which suppresses BTN3A1 binding, and also a second mechanism whereby the Vγ9Vδ2⁺ TCR-binding epitopes of BTN2A1 and BTN3A1 are partnered to each other in cis on the cell surface of APCs. The ability of the Lys53δ-Ala mutant TCR to induce Vγ9Vδ2⁺ T cell autoactivation and elevated BTN3A1 reactivity suggests that circumvention of the Lys53δ side chain might enable BTN3A1 to engage an adjacent epitope, such as one incorporating Arg51δ, Glu52δ and/or Lys108γ. Since BTN2A1 and BTN3A1 are both ligands of the Vγ9Vδ2⁺ TCR, yet are also direct interactants with each other, this may ensure that both ligands remain in an off-state, yet proximal to one another such that upon pAg triggering, the conversion of the complex into a stimulatory form is rapid and efficient. While the significance of the BTN2A1 V- and head-to-tail dimers remains to be tested, they are reminiscent of the reported BTN3A1 V- and head-to-tail dimers (A. Palakodeti et al., The molecular basis for modulation of human Vgamma9Vdelta2 T cell responses by CD277/butyrophilin-3 (BTN3A)-specific antibodies. 287, 32780-32790 (2012); S. Gu et al., Phosphoantigen-induced conformational change of butyrophilin 3A1 (BTN3A1) and its implication on Vgamma9Vdelta2 T cell activation. Proceedings of the National Academy of Sciences of the United States of America 114, E7311-E7320 (2017)). The stoichiometry of the stimulatory BTN2A1 and BTN3A1 complex, and the role of pAg, needs to be addressed in future studies.

The data provide three lines of evidence that BTN3A1 is a direct ligand of the Vγ9Vδ2⁺ TCR. Firstly, treatment of BTN3A1-transfected (but not parental) human or mouse APCs with agonist BTN3A mAb clone 20.1 bind Vγ9Vδ2⁺ TCR tetramers, and do so via a separate Vγ9Vδ2⁺ TCR epitope compared to BTN2A1-binding.

Secondly, recombinant BTN2A1-BTN3A1 complexes bind Vγ9Vδ2⁺ TCR-transfected cells in a way that co-depends on these same dual epitopes. Lastly, the co-binding by BTN2A1-BTN3A1 complexes was recapitulated in biophysical assays, thus excluding the role of any alternative ligands in binding Vγ9Vδ2⁺ TCR. Together, these observations indicate that whilst membrane-bound full-length BTN3A1 can bind Vγ9Vδ2⁺ TCR, a soluble form of the BTN3A1 ectodomain cannot do so unless BTN2A1 is also present, perhaps due to the requirement for a conformational change. Whether BTN2A1 induces a conformational change in BTN3A1, or vice versa, is unclear. Recombinant BTN2A1-BTN3A1 ectodomain complexes bound Vγ9Vδ2⁺ TCR with a similar affinity to BTN2A1 alone, suggesting that the energetic penalty of having BTN2A1 and BTN3A1 co-liganded to each other is offset by the gain in affinity achieved by having two complementary ligands. Indeed, the enhanced binding affinity of a BTN2A1-BTN3A1 Glu106 complex supports this conclusion, and further, also suggests that a single molecule of Vγ9Vδ2⁺ TCR can simultaneously co-bind both ligands.

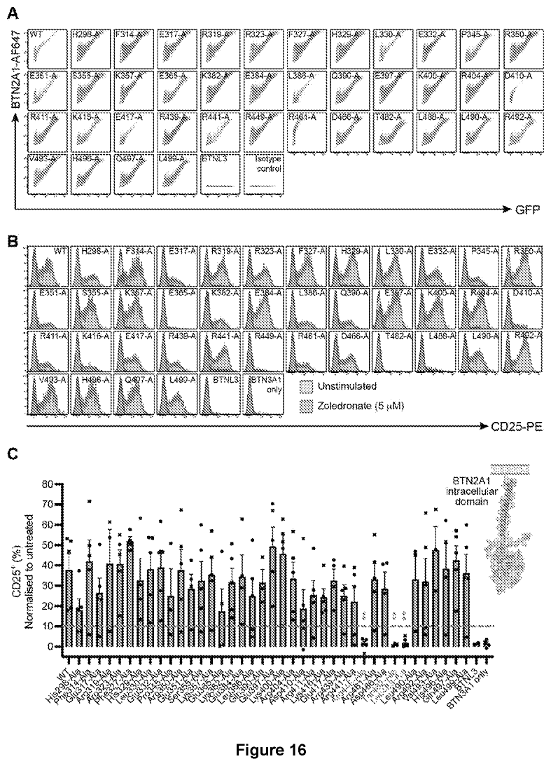

The intracellular domains of BTN2A1 and BTN3A1 are both required for pAg-induced activation of Vγ9Vδ2⁺ T cells (M. Rigau et al., Butyrophilin 2A1 is essential for phosphoantigen reactivity by gammadelta T cells. Science 367, (2020); C. E. Cano et al., BTN2A1, an immune checkpoint targeting Vgamma9Vdelta2 T cell cytotoxicity against malignant cells. Cell Rep 36, 109359 (2021)), and the BTN2A1-BTN3A1 interaction is enhanced by pAg (C. E. Cano et al., BTN2A1, an immune checkpoint targeting Vgamma9Vdelta2 T cell cytotoxicity against malignant cells. Cell Rep 36, 109359 (2021)). One interpretation of these findings is that pAg induces an association between the BTN3A1 and BTN2A1 intracellular domains. In support of this hypothesis, the present inventors identified three residues within the BTN2A1 intracellular domain—two in the C-terminal cytoplasmic tail (Thr482 and Leu488) and one in the B30.2 domain (Arg449)—that are critical for the activation of Vγ9Vδ2⁺ T cells (FIG. 16). Association of the intracellular domains may result in torsional forces that propagate through the rigid coiled-coil domains towards the ectodomains of the BTN complex. This might then enable the Vγ9Vδ2 TCR to first engage BTN2A1 with high affinity, and subsequently Vδ2/Vγ9 binding BTN3A1 to convey the presence of pAg. Collectively, our findings reveal a fundamentally different mode of immune activation and associated regulatory mechanisms underpin γδ T cell immunity compared to as T cells.

### BTN2A1-BTN3A1 (Glu106-Ala)-Zipper Binds to Vδ2+ Cells

To determine whether BTN2A1-BTN3A1 complexes could bind to primary Vδ2+ cells, Vδ2+ cells purified from the blood of a healthy donor that were pre-expanded with anti-CD3 and anti-CD28 mAb were stained with BTN2A1-BTN3A1 complex tetramers. The BTN2A1-BTN3A1-zipper complex (high affinity zippers with long linker) reacted with a minor subset of Vδ2+ cells (FIG. 17A), although this reactivity was enhanced by genetic modification of Vδ2+ cells to induce a Lys53δ-Ala mutation. However, the BTN2A1-BTN3A1 Glu106-Ala-zipper complex reacted very strongly with all Vδ2+ cells, even those that contained the parental Lys53δ residue (FIG. 17B). This indicates that a complex of BTN2A1-BTN3A1 wherein the BTN3A1 contains a Glu106-Ala mutation, induces binding to γδTCR. Conversely, the BTN2A1 Gly102-Cys-BTN3A1 Asp103-Cys-zipper complex failed to react with Vδ2+ cells (FIG. 17C), indicating that BTN2A1-BTN3A1 complex in a “locked” or closed conformation cannot bind to γδTCR. Thus, whilst the BTN2A1-BTN3A1 complex exhibits some reactivity for Vδ2+ cells, this reactivity is enhanced by a Glu106-Ala mutation within BTN3A1. Of note, the BTN2A1 (WT)-BTN3A1 Glu106-Ala-zipper was notably associated with disruption of the association between the IgV domains of BTN2A1 and BTN3A1 (FIG. 8H), therefore implying that this mutation alters the structure of the BTN2A1-BTN3A1 complex in such a way that it enhances its reactivity to the γδTCR.

### Phosphoantigen Independent Binding

To determine if BTN2A1 and BTN3A1 ectodomains can induce Vδ2 T cell activation in the absence of phosphoantigen, immobilized ectodomains were tested for their ability to activate Vδ2 cells in vitro. Whilst immobilized BTN2A1 and BTN3A1 only lead to marginal or no activation of Vδ2+ cells, respectively, the combination of BTN2A1 plus BTN3A1 induced clear activation. Further, tethered BTN2A1-BTN3A1 ectodomains with C-terminal zippers were also stimulatory in the same assay; however, cleavage of the zippers abrogated or reduced their stimulatory effect. Accordingly, BTN2A1 plus BTN3A1 are necessary and sufficient to mediate the activation of Vδ2+ cells, obviating the requirement for addition of exogenous phosphoantigen.

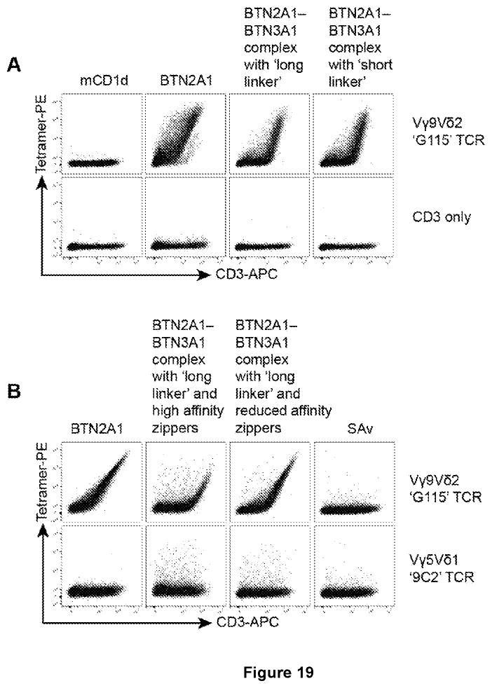

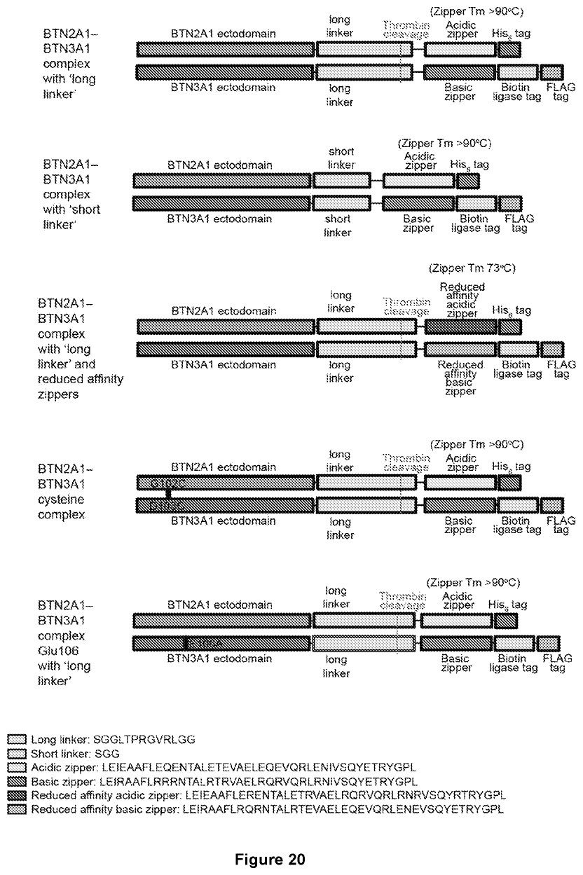

To determine if BTN2A1-BTN3A1 complexes tethered to each other with zippers connected via a shorter C-terminal linker retained reactivity to γδTCR, compared to the original 13-mer Gly/Ser-rich linker, the inventors produced BTN2A1-BTN3A1 complex wherein the zipper domains were fused to the C-termini of the BTN molecules with a short linker that was three residues in length (sequence: SGG). This BTN2A1-BTN3A1 ‘short linker’ complex retained reactivity to surface-expressed Vγ9Vδ2⁺ TCR expressed on NIH-3T3 cells (FIG. 19A), suggesting that alterations to the linker motif did not impair reactivity of the BTN2A1-BTN3A1 complex to Vγ9Vδ2⁺ TCR.

Next, the zipper motifs were modified such that they reduced or eliminated homodimerization, but still retained the ability to heterodimerize (J. R. Moll et al., Designed heterodimerizing leucine zippers with a ranger of pls and stabilities up to 10(−15) M. Protein Sci 10, 649-655 (2001)). This did not impair reactivity to Vγ9Vδ2⁺TCR (FIG. 19B), suggesting that the zipper fusion motifs can be modified significantly without abrogating BTN2A1-BTN3A1 complex reactivity to Vγ9Vδ2⁺ TCR. These data indicate that BTN2A1 and BTN3A1 molecules tethered to each other in several different ways, enable binding to Vγ9Vδ2⁺ TCR.

### Mutation of Either BTN2A1 Ser44-Arg or BTN3A1 Glu106-Ala Enhance Vγ9Vδ2 TCR Binding to BTN2A1-BTN3A1 Complex

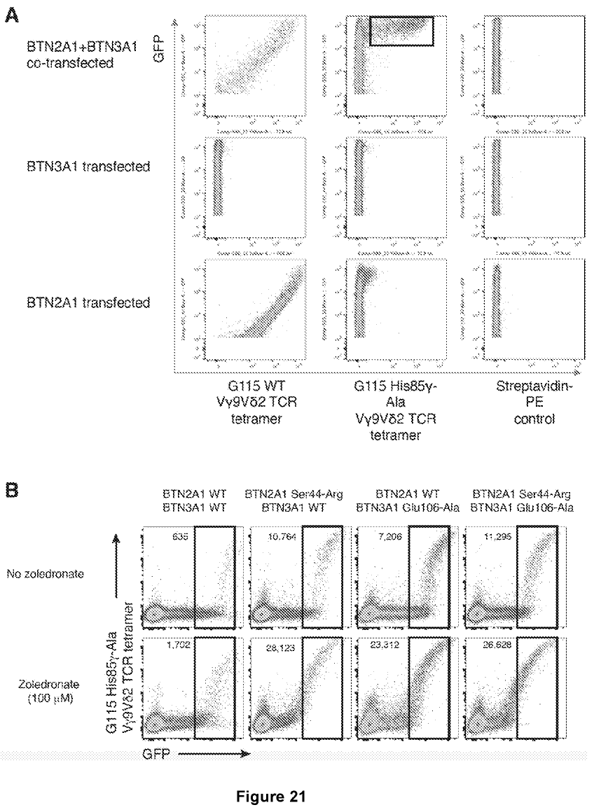

The inventors next tested if mutations to either BTN2A1 or BTN3A1 can influence binding of Vγ9Vδ2 TCR to BTN2A1-BTN3A1 complex that is expressed on the cell surface. Since Vγ9Vδ2 TCR tetramers can bind surface-expressed BTN2A1 independently of BTN3A1 (FIG. 3), the inventors devised a strategy to circumvent the overwhelming influence of BTN2A1. Here, rather than staining the BTN-expressing cells with WT Vγ9Vδ2+ TCR tetramers, the inventors instead used G115 Vγ9Vδ2+ TCR tetramers with a His85γ-Ala mutation, which greatly reduces the TCR interaction with BTN2A1 (FIG. 4C). Using this strategy, co-expression of BTN2A1 and BTN3A1, but not either molecule alone, facilitated binding of His85γ-Ala Vγ9Vδ2+ TCR tetramers. Thus, His85γ-Ala Vγ9Vδ2+ TCR interacts with BTN2A1-BTN3A1 complex in a manner that depends on the presence of both BTN2A1 and BTN3A1 on the cell surface (FIG. 21A).

The inventors discovered a mutation within BTN3A1, Glu106-Ala, that enhances the ability of soluble BTN2A1-BTN3A1 complex to interact with Vγ9Vδ2+ TCR (FIG. 17). Consistent with this notion, mouse NIH-3T3 cells co-transfected with genes encoding BTN2A1 (WT) and BTN3A1 (Glu106-Ala) interacted with His85γ-Ala Vγ9Vδ2+ TCR tetramers more strongly than cells co-transfected with BTN2A1 (WT) and BTN3A1 (WT) (FIG. 21B). This equated to an 11±0.1-fold increase in TCR tetramer binding to GFP+ cells, averaged across two independent experiments. Surprisingly, the inventors also discovered that mutation of Ser44-Arg within BTN2A1 resulted in a similar effect, whereby cells co-transfected with BTN2A1 (Ser44-Arg) and BTN3A1 (WT) interacted with Vγ9Vδ2+ TCR tetramers more strongly than cells co-transfected with BTN2A1 (WT) and BTN3A1 (WT) (FIG. 21B). This equated to a 14±3-fold increase in TCR tetramer binding to GFP+ cells. Cells co-expressing both mutants, i.e. BTN2A1 (Ser44-Arg) and BTN3A1 (Glu106-Ala) exhibited a 14±4-fold increase in TCR tetramer binding to GFP+ cells, as compared to WT controls (FIG. 21B). Therefore, mutations within or near to the BTN2A1-BTN3A1 binding interface that is located on their IgV domains can facilitate enhanced interaction with Vγ9Vδ2+ TCR. Additionally, treatment of cells with the aminobisphosphonate zoledronate further enhanced the interaction between BTN2A1-BTN3A1 complex and His85γ-Ala Vγ9Vδ2+ TCR tetramers, indicating that elevated intracellular phosphoantigen increases the affinity of the interaction between BTN2A1-BTN3A1 complex and Ala Vγ9Vδ2+ TCR (FIG. 21B).

### Methods

### Human Samples

Healthy donor blood derived human peripheral blood cells (PBMCs) from male and female donors were obtained from the Australian Red Cross Blood Service under ethics approval 17-08VIC-16 or 16-12VIC-03, with ethics approval from University of Melbourne Human Ethics Sub-Committee (1035100) and isolated via density gradient centrifugation (Ficoll-Paque PLUS GE Health care) and red blood cell lysis (ACK buffer, produced in-house).

### Cell Lines

Jurkat (JR3-T3.5), LM-MEL-75, HEK293T and NIH-3T3 cells were existing tools in the lab and were maintained in RPMI-1640 (Invitrogen) supplemented with 10% (v/v) FCS (JRH Biosciences), penicillin (100 U/ml), streptomycin (100 μg/ml), Glutamax (2 mM), sodium pyruvate (1 mM), nonessential amino acids (0.1 mM) and HEPES buffer (15 mM), pH 7.2-7.5 (all from Invitrogen Life Technologies), plus 50 μM 2-mercaptoethanol (Sigma-Aldrich) (complete RMPI). Expi293F cells were purchased from ThermoFisher (Cat. No. A14527) and maintained in Expi293 Expression Medium (ThermoFisher, A1435101). γδ T cell isolation and expansion

In some experiments γδ T cells were enriched by MACS using either anti-γδTCR-PECy7 followed by anti-phycoerythrin-mediated magnetic bead purification. After enrichment CD3⁺ Vδ2⁺γδ T cells were further purified by sorting using an Aria III (BD). Enriched γδ T cells were stimulated in vitro for 48 h with plate-bound anti-CD3e (OKT3, 10 μg/ml, Bio-X-Cell), soluble anti-CD28 (CD28.2, 1 μg/ml, BD Pharmingen), phytohemagglutinin (0.5 μg/ml, Sigma) and recombinant human IL-2 (100 U/ml, PeproTech), followed by maintenance with IL-2 for 14-21 d. Cells were cultured in complete medium consisting of a 50:50 (v/v) mixture of AIM-V (Thermo Fisher) and RPMI-1640 supplemented with 10% (v/v) FCS, penicillin (100 U/ml), streptomycin (100 μg/ml), Glutamax (2 mM), sodium pyruvate (1 mM), nonessential amino acids (0.1 mM) and HEPES buffer (15 mM), pH 7.2-7.5, plus 50 μM 2-mercaptoethanol.

### Flow Cytometry

To examine the capacity of γδTCR tetramers to bind to BTN molecules, NIH-3T3 cells were transfected with BTN2A1, BTN3A1 or control BTNL3 in pMIG (a gift from D. Vignali (Addgene plasmid #52107) (21) using ViaFect® (Promega) in OptiMEM™ (Gibco, Thermo-Fisher). 48 h following transfection, cells were harvested with trypsin, filtered through a 30 or 70 μm cell strainer, and incubated with anti-BTN3A antibody (clone 20.1) or IgG1,κ isotype control (clone MOPC-21, BioLegend; or BM4-1, a gift from CSL Limited) at 5 μg/mL for 15 min at room temperature. Cells were then stained with PE-labelled γδTCR tetramers (produced in house, see below), or control PE-conjugated streptavidin, at 5 μg/mL for 30 min at room temperature. The median fluorescence intensity (MFI) of γδTCR tetramer interacting with BTN proteins was examined on gated GFP⁺ cells by flow cytometry. For γδTCR tetramer staining, data were excluded if BTN3A1 mutant protein levels were >2-fold lower than wild-type BTN3A1, as determined by anti-BTN3A mAb staining. To examine the capacity of BTN tetramers to bind to γδTCRs, HEK293T cells were co-transfected with γδTCR genes in pMIG using FuGENE® HD (Promega) in OptiMEM™ plus 2A-linked CD3εδγζ in pMIG. 48 h following transfection, cells were collected by pipetting, filtered through a 30 or 70 μm cell strainer, and stained with anti-CD3E antibody for 15 min at 4° C. In some experiments, zoledronate was added overnight prior to cell harvest. Cells were then stained with anti-γδTCR, anti-TCR Vδ2 as well as PE-labelled BTN tetramers (produced in house, see below), PE-labelled control mouse CD1d ectodomain tetramers (loaded with α-GalCer and produced in house, see below), or control PE-conjugated streptavidin (BD), for 30 min at 4° C. The MFI of BTN tetramer on gated CD3⁺ GFP⁺ cells was measured by flow cytometry. In other assays, human peripheral blood-derived cells were stained with 7-aminoactinomycin D (7-AAD, Sigma) or LIVE/DEAD® viability markers (ThermoFisher) plus antibodies against: CD3ε, γδTCR, TCR Vδ2, CD45, CD25, CD69, and/or isotype controls (IgG1,κ clone MOPC-2) in various combinations (Table 6). All data were acquired on an LSRFortessa™ II (BD) and analyzed with FACSDiva and FlowJo (BD) software. All samples were gated to exclude unstable events, doublets and dead cells using time, forward scatter area versus height, and viability dye parameters, respectively.

TABLE 6
Antibodies used for Flow Cytometry and T cell expansion.

| Target | Clone | Fluorophore | Cat. No. | Company |
|---|---|---|---|---|
| IgG1, κ | BM4-1 | AlexaFluor647 | | In house |
| IgG2a, κ | BM4-2a | AlexaFluor647 | | In house |
| IgG1, κ | MOPC-21 | PE | 400112 | BioLegend |
| IgG1, κ | MOPC-21 | Purified | 400102 | BioLegend |
| BTN2A1 | 259 | PE | | In house |
| BTN3A | 103.2 | PE | | In house |
| BTN3A | 20.1 | AlexaFluor647 | | In house |
| BTN3A | 20.1 | Purified | | In house |
| CD3e | UCHT1 | BUV395 | 563546 | BD Pharmingen |
| CD3e | OKT3 | Purified | BE0001-2 | Bio-X-Cell |
| CD25 | M-A251 | PE | 555432 | BD Pharmingen |
| CD28 | CD28.2 | NA/LE | 567117 | BD Pharmingen |
| CD45 | HI30 | AlexaFluor700 | 304024 | BioLegend |
| CD69 | FN50 | APC | 555533 | BD Pharmingen |
| γδ TCR | 11F2 | PEcy7 | 655410 | BD Pharmingen |
| Vδ2 TCR | B6 | BV711 | 331412 | BioLegend |

### Generation of BTN2A.BTN3A-Knockout Cells

HEK293T cells were nucleofected with Cas9/RNP complexes and two guide RNAs, one targeting the intronic region directly upstream of BTN3A2 (5′-AACTTTCACCTACAAACCGC; SEQ ID NO: 55) and one downstream of BTN2A1 (5′-GAACCCTGACTGAAACGATC; SEQ ID N:56). Guides were designed using the Broad Institute CRISPick web tool (H. K. Kim et al., Deep learning improves prediction of CRISPR-Cpf1 guide RNA activity. Nat Biotechnol 36, 239-241 (2018)). After seven days in culture, RNP⁺ cells were bulk-sorted (FACS Aria Ill) and after another round of culture were single cell-sorted. To verify excision of the BTN locus, genotyping of the expanded clones was performed using PCR primers targeting BTN3A2, BTN2A1 and the excised locus (Table 7).

TABLE 7
Primers used for PCR and site-directed
mutagenesis.

| | | |
|---|---|---|
| | | SEQ |
| | | ID |
| Name | Sequence (5′ to 3′) | NO: |

Vδ2 site-directed mutagenesis:

| | | |
|---|---|---|
| E3A_F | TCTGCCATCGcGCTGGTGCCT | 57 |
| E3A_R | CATCACGCCAGCCCAAAAC | 58 |
| Q9A_F | GCCTGAACACgcGACCGTGCCTG | 59 |
| Q9A_R | ACCAGCTCGATGGCAGAC | 60 |
| T20A_F | CGTGCCAGCCgCACTGCGGTG | 61 |
| T20A_R | CCAATAGACACAGGCACGGTCTGG | 62 |
| K26A_F | GTGTAGCATGgcGGGCGAAGCCATCGG | 63 |
| K26A_R | CGCAGTGTGGCTGGCACG | 64 |
| K53A_F | CTACAGAGAGgcGGACATCTACG | 65 |
| K53A_R | ATGAAGGTCATGGTGTTG | 66 |
| D54A_F | AGAGAGAAGGcCATCTACGGC | 67 |
| D54A_R | GTAGATGAAGGTCATGGTG | 68 |
| K61A_F | CCCTGGCTTCgcGGACAACTTC | 69 |
| K61A_R | CCGTAGATGTCCTTCTCTC | 70 |
| D62A_F | GGCTTCAAGGcCAACTTCCAG | 71 |
| D62A_R | AGGGCCGTAGATGTCCTT | 72 |
| Q65A_F | GGACAACTTCgcGGGCGACATCGACATTG | 73 |
| Q65A_R | TTGAAGCCAGGGCCGTAG | 74 |
| D69A_F | GGCGACATCGcCATTGCCAAG | 75 |
| D69A_R | CTGGAAGTTGTCCTTGAAGC | 76 |
| I70A_F | CGACATCGACgcTGCCAAGAACC | 77 |
| I70A_R | CCCTGGAAGTTGTCCTTG | 78 |
| K72A_F | CGACATTGCCgcGAACCTGGCC | 79 |
| K72A_R | ATGTCGCCCTGGAAGTTG | 80 |
| K78A_F | GGCCGTGCTGgcGATTCTGGCC | 81 |
| K78A_R | AGGTTCTTGGCAATGTCG | 82 |
| L80A_F | GCTGAAGATTgcGGCCCCTAGCGAG | 83 |
| L80A_R | ACGGCCAGGTTCTTGGCA | 84 |
| R114A_F | CAAGGGCACCgcAGTGACCGTG | 85 |
| R114A_R | CCGAAGATCAGCTTGTCG | 86 |

Genotyping:

| | | |
|---|---|---|
| BTN3A2_F | TGCAAAAGCACTGCAGACAC | 87 |
| BTN3A2_R | AGCCAGCCTGAGGGTAACTT | 88 |
| BTN2A1_F | TTTCTTAGGATTCTGCCCGCC | 89 |
| BTN2A1_R | TCCTTAGGGCCCAGGACTAT | 90 |
| BTN2A- | ACCAGAAGTACCACTGGCTT | 91 |
| 3A_locus_F | | |
| BTN2A- | AACCCTGTTTTCTGCCTTAACA | 92 |
| 3A_locus_R | | |

### Jurkat Assays

2.5×10⁴ APCs (LM-MEL-75) cells were plated per well of a 96-well plate and incubated overnight, before 2×10⁴ G115 mutant-expressing J.RT3-T3.5 (Jurkat) cells±zoledronate (40 μM) were added for 20 h. CD69 expression was measured by flow cytometry on GFP⁺ Jurkat cells. A panel of 15 single-residue alanine (Ala) mutants, each within in the Vδ2 domains of the Vγ9Vδ2⁺ G115 TCR were generated by either site-directed mutagenesis using the primers listed in Table 5, or by cloning of gene fragments (IDT). Primers (IDT) were phosphorylated (PNK, NEB) followed by 25 cycles of PCR using KAPA HiFi master mix (KAPA Biosystems) using G115 WT TCR in pMIG as template, and PCR product was digested with Dpnl (NEB) and in some cases ligated with T4 DNA ligase (NEB). Construct sequences were verified by Sanger sequencing prior to use.

### γδ T Cell Functional Assays

For co-culture assays, NIH-3T3 cells were transfected with BTN2A1 in combination with wild-type or mutant BTN3A1, or separately with control BTNL3 and BTNL8 in pMIG with ViaFect® in OptiMEM™. 48 h following transfection, NIH-3T3 cells (3×10⁴) were harvested, transferred to 96-well plates and incubated with purified in vitro-expanded Vδ2⁺γδ T cells (2×10⁴) for 24 h±zoledronate (5 μM). γδ T cell activation was determined by CD25 upregulation using flow cytometry. For γδ T cell functional assays, samples were excluded if transfection efficiency was less than 10%.

### Detection of Forster Resonance Energy Transfer

NIH-3T3 cells were transfected with BTN2A1 in combination with wild-type or mutant BTN3A1, or control BTN2A1 transfected with PDL2/BTN3A1 transfected with CD80, in pMIG with ViaFect® in OptiMEM™. 48 h following transfection, NIH-3T3 cells (3×10⁴) were harvested with trypsin, filtered through 30-70 μm cell strainers, and stained with anti-BTN2A1-AlexaFluor647 (clone 259) and BTN3A-PE (clone 103.2) or isotype controls (clones BM4-2a and MOPC-21, respectively) for 30 min at 4° C. The frequency of cells identified as FRET⁺ was examined on gated GFP⁺ AlexaFluor647⁺ PE⁺ NIH-3T3 cells. For FRET experiments, data were excluded if BTN3A1 mutant protein levels were >2-fold lower than BTN3A1 WT as determined by anti-BTN3A (clone 103.2) mAb staining.

### Production of Soluble Proteins and Tetramers

Soluble human BTN2A1-BTN3A1 ectodomains including those containing ‘short’ or ‘long’ C-terminal linkers, ‘high’ and ‘reduced’ affinity C-terminal zippers, a BTN3A1 Glu106-Ala mutation, or containing BTN2A1 Gly102-Cys/BTN3A1 Asp103-Cys mutations, or, or alternatively BTN2A1 ectodomains containing a C-terminal Cys (Cys247) and an acidic or basic leucine zipper, along with soluble γδTCRs, BTN1A1, BTN2A1 lacking Cys247, BTN3A1, BTN3A1 IgV domain, and mouse CD1d ectodomains were all expressed by transient transfection (1:1 DNA ratio when co-transfecting with two constructs) of mammalian Expi293F or MGAT1^{null} (GNTI) HEK-293S cells using ExpiFectamine or PEI, respectively, with pHL-see vector DNA encoding constructs with C-terminal biotin ligase (AviTag™) and Hise tags (A. R. Aricescu et al., A time- and cost-efficient system for high-level protein production in mammalian cells. Acta Crystallogr D Biol Crystallogr 62, 1243-1250 (2006)). Protein was purified from culture supernatant using immobilized metal affinity chromatography (IMAC) and gel filtration, and enzymatically biotinylated using BirA (produced in-house). Proteins were re-purified by size exclusion chromatography and stored at −80° C. Biotinylated proteins were tetramerized with streptavidin-PE (BD) at a 4:1 molar ratio.

### Structure Determination

BTN2A1 and G115 γδTCR (either WT or Lys53δ-Ala) were mixed at a 1:1 molar ratio (15 mg/ml in Tris-buffered saline pH 8) and crystallized at 20° C. in 20% polyethylene glycol (PEG) 3350/0.2 M sodium malonate/malonic acid pH 7.0; apo BTN2A1 (10 mg/ml in Tris-buffered saline pH 8) was crystallized at 20° C. in 1.65 M ammonium sulfate/2% (v/v) PEG 400/0.1 M HEPES pH 8; and BTN2A1-BTN3A1-zippered complex (1 mg/ml in Tris-buffered saline pH 8) was crystallized at 20° C. in 6% (w/v) PEG 6000/0.1 M magnesium sulfate/0.1 M HEPES pH 6 by sitting drop vapour diffusion (C3 facility, CSIRO, Australia). Crystals of BTN2A1-G115 γδTCR, apo BTN2A1 and BTN2A1-BTN3A1-zippered complex were flash frozen in mother liquor plus 27.5% (w/v) PEG/0.2 M sodium malonate, 1.8 M ammonium sulfate/2% (v/v) PEG 400/15% (v/v) glycerol, or in well solution plus 20% (v/v) glycerol, respectively. Data were collected at 100 K using the MX2 (3|D1) beamline at the Australian Synchrotron with an Eiger detector operating at 100 Hz. Data were integrated using iMosflm version 7.3.0 (T. G. Battye, L. Kontogiannis, O. Johnson, H. R. Powell, A. G. Leslie, iMOSFLM: a new graphical interface for diffraction-image processing with MOSFLM. Acta Crystallogr D Biol Crystallogr 67, 271-281 (2011)) and, in the case of BTN2A1-G115 γδTCR, processed using the Aimless package in CCP4, or in the case of apo BTN2A1 and BTN2A1-BTN3A1-zippered complex, subjected to the STARANISO Server (Global Phasing Ltd.) (staraniso.globalphasing.org/cgi-bin/staraniso.cgi) to perform an anisotropic cut-off and to apply an anisotropic correction to the data. Apo BTN2A1 was solved by molecular replacement using the IgV and IgC domains of bovine BTN1A1 as separate search ensembles (PDB code 4HH8 (A. Eichinger, I. Neumaier, A. Skerra, The extracellular region of bovine milk butyrophilin exhibits closer structural similarity to human myelin oligodendrocyte glycoprotein than to immunological BTN family receptors. Biol Chem, (2021))); BTN2A1-G115 γδTCR was solved by molecular replacement using G115 TCR (PDB code 1HXM (T. J. Allison, C. C. Winter, J. J. Fournie, M. Bonneville, D. N. Garboczi, Structure of a human gammadelta T-cell antigen receptor. Nature 411, 820-824 (2001))) and monomeric BTN2A1; BTN2A1-BTN3A1-zippered complex was solved by molecular replacement using monomeric BTN2A1, and BTN3A1 (from PDB code 4F80 (A. Palakodeti et al., The molecular basis for modulation of human Vgamma9Vdelta2 T cell responses by CD277/butyrophilin-3 (BTN3A)-specific antibodies. 287, 32780-32790 (2012)), with Phaser (P. D. Adams et al., PHENIX: a comprehensive Python-based system for macromolecular structure solution. Acta Crystallogr D Biol Crystallogr 66, 213-221 (2010)). Refinement of BTN2A1-G115 γδTCR was performed by iterative rounds of model building into experimental maps in Coot and refinement with Buster version 2.10.4 (Global Phasing), using non-crystallographic symmetry (NCS) restraints applied to BTN2A1, excluding residues at the TCR-binding interface (O. S. Smart et al., Exploiting structure similarity in refinement: automated NCS and target-structure restraints in BUSTER. Acta Crystallogr D Biol Crystallogr 68, 368-380 (2012)). Refinement of apo BTN2A1 and BTN2A1-BTN3A1-zippered complex were similarly restrained against the unliganded copy of BTN2A1 from BTN2A1-G115 γδTCR, or BTN3A1 from 4F80, excluding residues at the interfaces. The structural models were analyzed with the CCP4 suite version 7.1 (M. D. Winn et al., Overview of the CCP4 suite and current developments. Acta Crystallogr D Biol Crystallogr 67, 235-242 (2011)). Molecular figures were generated with PyMOL (Schrödinger). Cation-rr interactions were determined as described (32). Angles were calculated between the center of masses of the Ig domains, or in some cases by the intersection of two planes, each defined by three points. Modelling in FIG. S10 was performed using AlphaFold 2.0 (J. Jumper et al., Highly accurate protein structure prediction with AlphaFold. Nature 596, 583-589 (2021)).

### Surface Plasmon Resonance

SPR experiments were conducted at 25° C. on a Biacore T200 instrument (GE Healthcare) using 10 mM HEPES-HCl (pH 7.4), 150 mM NaCl, 3 mM EDTA, and 0.05% Tween 20 buffer. Biotinylated BTN ectodomains were immobilized to 1,500-2,000 resonance units (RU) on a Biacore sensor chip SA pre-immobilized with streptavidin. Soluble BTN molecules or G115 γδTCR were two-fold serially diluted and simultaneously injected over test and control surfaces at a rate of 30 μl/min. After subtraction of data from the control flow cell (BTN1A1) and blank injections, interactions were analyzed using Biacore T200 evaluation software (GE Healthcare), Scrubber (Biologic) and Prism version 9 (GraphPad), and equilibrium dissociation constants were derived at equilibrium.

### Electron Microscopy

Soluble BTN2A1 Gly130-Cys-BTN3A1 Asp132-Cys complex was enzymatically digested with thrombin to remove C-terminal leucine zippers, repurified by size exclusion and anion exchange chromatography, and spotted onto glow-discharged 400 mesh thin carbon-coated copper grids at 380 μg/ml in TBS for 30 seconds, followed by negative staining with 2% w/v uranyl acetate. Grids were observed on a FEl Tecnai F30 (Eindhoven, NL) 300 kV transmission electron microscope at a nominal magnification of ×52,000. Seventeen micrographs were acquired on a CETA (Thermofisher, USA) camera with a 3.7 Å pixel size. Particles were picked using blob picking followed by 2D class averaging in cryoSPARC (A. Punjani, J. L. Rubinstein, D. J. Fleet, M. A. Brubaker, cryoSPARC: algorithms for rapid unsupervised cryo-EM structure determination. Nat Methods 14, 290-296 (2017)), with 10,238 particles contributing to the final set of 2D class averages.

### Statistical Analysis

γδ T cell functional assays were analysed by 2-way ANOVA with Sidak's correction when comparing γδ T cell activation (CD25⁺) with and without treatment across various BTN mutants. All independent datapoints are biological replicates.

### Germline Modification of Vδ2 of Purified γδ T Cells

Primary Vδ2+ T cells were enriched from PBMC using anti-PE magnetic beads (Miltenyl), following staining with anti-PE-cy7 γδTCR mAb (clone: 11F2 at a 1:20 dilution). Cells were stimulated on plates coated with anti-CD3 (clone: UCHT1 at a concentration of 5 μg/mL) and soluble anti-CD28 (clone: T44 at a concentration of 10 μg/mL) in media containing 10 U/mL of IL-2. After 3 days, cells were nucleofected with guide RNA targetting the CDR2δ region, proximal to Lys53, of the Vδ2 chain (guide RNA #1: GACTTTCATATACCGAGAAA (SEQ ID NO:93); guide RNA #2: GGCCATAGATGTCCTTTTCT (SEQ ID NO:94) plus a HDR single-stranded oligonucleotide template that encoded a Lys53δ-Ala point mutation (Alt-R HDR oligonucleotide sequence: A*C*CTT GGA AAT TGT CTT TGA AAC CAG GGC CAT AGA TGT CCG CCT CTC GGT ATA TGA AAG TCA TTG TGT TAC CTT GGG TCT T*C*C, where * represents a phosphorothioate bond (SEQ ID NO:95) Integrated DNA Technologies). Cells were maintained for a further 7 days, at which point they were screened by flow cytometry. Tumour killing assays were performed 10 days after nucleofection.

### Vδ2 T Cell Activation in the Absence of Phosphoantigen

BTN2A1, BTN3A1, or BTN2A1-BTN3A1-zipper complex, or control proteins were immobilized onto 96 well tissue culture plates overnight at 4 degrees at 10 μg/mL. In one group BTN2A1-BTN3A1-zipper complex was also pre-incubated overnight with thrombin in order to cleave the zippers off. Plates were washed to remove unbound ligand and purified pre-expanded Vδ2⁺ cells were added, and CD25 expression was measured on gated cells after an overnight co-culture.

1. A BTN2-BTN3 heteromeric complex comprising:
(i) a first polypeptide comprising an IgV-like domain of BTN2 or a Vγ9+ TCR binding portion thereof and a first heterologous C-terminal dimerization peptide,
(ii) a second polypeptide comprising an IgV-like domain of BTN3 or a Vδ2+ TCR binding portion thereof and a second heterologous C-terminal dimerization peptide which specifically dimerizes with the first heterologous C-terminal dimerization peptide to form a heterodimerization domain,
wherein the BTN2-BTN3 heteromeric complex binds to a Vγ9Vδ2⁺ TCR.

2. The BTN2-BTN3 heteromeric complex of claim 1, wherein the heterodimerisation domain is a coiled coil domain, optionally wherein the dimerisation peptides are c-jun and c-fos dimerisation peptides.

3. (canceled)

4. The BTN2-BTN3 heteromeric complex of claim 1, wherein the heterodimerisation domain comprises a disulphide bond, optionally wherein the dimerisation peptides comprise a Cys residue.

5. (canceled)

6. A BTN2-BTN3 heteromeric complex comprising:
(i) a first polypeptide comprising an IgV-like domain of BTN2 or a Vγ9+ TCR binding portion thereof,
(ii) a second polypeptide comprising an IgV-like domain of BTN3 or a Vδ2+ TCR binding portion thereof, and
(iii) a peptide linker linking said first and second polypeptides into a single chain polypeptide,
wherein the BTN2-BTN3 heteromeric complex binds to a Vγ9Vδ2+ TCR.

7. The BTN2-BTN3 heteromeric complex of claim 6, wherein the single chain linker comprises from about 15 to 40 amino acids, or from about 50 to 150 amino acids.

8. (canceled)

9. The BTN2-BTN3 heteromeric complex of claim 6, wherein at least about 80% of the residues of the linker are glycine or serine residues, and/or the linker does not comprise any proline residues.

10. (canceled)

11. The BTN2-BTN3 heteromeric complex of claim 1, wherein the heteromeric complex binds to a Vγ9Vδ2⁺ TCR independent of phosphoantigen, and/or binds a Vγ9Vδ2⁺ TCR with enhanced binding compared to BTN2 or BTN3 alone, and/or induces or enhances Vγ9Vδ2⁺ TCR activation.

12. (canceled)

13. (canceled)

14. The BTN2-BTN3 heteromeric complex of claim 1, wherein the first polypeptide comprises an amino acid sequence that is at least 70% identical to the sequence shown in SEQ ID NO:1, and/or wherein the second polypeptide comprises an amino acid sequence that is at least 70% identical to the sequence shown in SEQ ID NO:2.

15. (canceled)

16. The BTN2-BTN3 heteromeric complex of claim 1, wherein the BTN2-BTN3 heteromeric complex is soluble, optionally wherein the first and second polypeptides lack a functional transmembrane domain and a cytoplasmic domain.

17. (canceled)

18. The BTN2-BTN3 heteromeric complex of claim 1, wherein one or both the first and second polypeptides comprise one or modified amino acid residues selected from the group consisting of: a glycosylated amino acid, a PEGylated amino acid, a farnesylated amino acid, an acetylated amino acid, a biotinylated amino acid, and an amino acid conjugated to a lipid moiety.

19. The BTN2-BTN3 heteromeric complex of claim 1, wherein one or both the first and second polypeptides, further comprise one or more purification sequences optionally selected from the group consisting of: an epitope tag, a FLAG tag, a polyhistidine sequence, and a GDT fusion.

20. The BTN2-BTN3 heteromeric complex of claim 1, wherein one or both the first and second polypeptides is glycosylated and has a glycosylation pattern obtainable from Expi293 cells.

21. The BTN2-BTN3 heteromeric complex of claim 1, wherein the BTN2-BTN3 heteromeric complex binds to activin, optionally to activin A.

22. (canceled)

23. A multivalent BTN2-BTN3 complex comprising two or more linked BTN2-BTN3 heteromeric complexes of claim 1.

24. A modified BTN3 or VS2⁺ TCR binding fragment thereof, wherein the modified BTN3 comprises an IgV-like domain, wherein the IgV-like domain comprises a modification at a position that corresponds to glutamic acid (E) 106 of the amino acid sequence shown in SEQ ID NO:2, optionally to a glutamic acid (E) to alanine (A) substitution at position 106, and/or optionally the IgV-like domain comprises an amino acid sequence having at least 70% identity to SEQ ID NO: 2.

25. (canceled)

26. (canceled)

27. The modified BTN3 or Vδ2⁺ TCR binding fragment thereof of claim 24, wherein the modified BTN3 binds to a Vδ2⁺ TCR independent of phosphoantigen, and/or binds to a Vδ2⁺ TCR with enhanced binding, and/or induces or enhances Vδ2⁺ TCR activation.

28. (canceled)

29. (canceled)

30. A modified BTN2 or Vγ9⁺ TCR binding fragment thereof, wherein the modified BTN2 comprises an IgV-like domain, wherein the IgV-like domain comprises a modification at a position that corresponds to serine (S) 44 of the amino acid sequence shown in SEQ ID NO: 1, optionally to a serine (S) to arginine (R) substitution at position 44, and/or optionally the IgV-like domain comprises an amino acid sequence having at least 70% identity to SEQ ID NO: 1.

31. (canceled)

32. (canceled)

33. (canceled)

34. (canceled)

35. (canceled)

36. One or more nucleic acids encoding the BTN2-BTN3 heteromeric complex of claim 1, optionally wherein the one or more nucleic acids comprise:
i) a nucleic acid sequence having at least 70% identity to SEQ ID NO: 5; and/or
ii) a nucleic acid sequence having at least 70% identity to SEQ ID NO: 6.

37. (canceled)

38. One or more vectors comprising one or more nucleic acids encoding the BTN2-BTN3 heteromeric complex of claim 1.

39. A host cell comprising the BTN2-BTN3 heteromeric complex of claim 1.

40. A method for activating γδ T cells that express a δ2⁺ TCR, the method comprising contacting the cells with the BTN2-BTN3 heteromeric complex of claim 1; and, optionally, administering the activated γδ T cells to a subject in need thereof.
